(19) United States
(12) Patent Application Publication
(10) Pub. No.: US 2024/0238403 A1
(43) Pub. Date: Jul. 18, 2024

(54) INFLUENZA VIRUS REPLICATION FOR VACCINE DEVELOPMENT

(71) Applicant: Wisconsin Alumni Research Foundation, Madison, WI (US)

(72) Inventors: Yoshihiro Kawaoka, Middleton, WI (US); Gabriele Neumann, Madison, WI (US); Lizheng Guan, Madison, WI (US)

(21) Appl. No.: 18/511,485

(22) Filed: Nov. 16, 2023

Related U.S. Application Data
(60) Provisional application No. 63/384,026, filed on Nov. 16, 2022.

(51) Int. Cl. A61K 39/145; C12N 7/00; A61K 39/00

(52) U.S. Cl. CPC A61K 39/145; C12N 7/00; A61K 2039/5256; A61K 2039/55; C12N 2760/16121; C12N 2760/16134; C12N 2760/16151

(57) ABSTRACT

As described herein, influenza A viruses were developed that replicate to high titers in cultured cells and/or embryonated chicken eggs. Mutations were identified that resulted in higher virus titers in cultured cells and/or embryonated chicken eggs, allowing more efficient influenza virus growth and more cost-effective vaccine production. Replication-enhancing residues include, but are not limited to, PB2 439H, PB1 577R, PB1 640V, M1 35R, PB1 62E, and/or PB1 624I, or combinations thereof.

### CROSS REFERENCE TO RELATED APPLICATIONS

This application is a continuation of U.S. Provisional Application Ser. No. 63/384,026, filed Nov. 16, 2022, the contents of which are specifically incorporated herein by reference in its entirety.

### STATEMENT OF GOVERNMENT SUPPORT

This invention was made with government support under HHSN272201400008C awarded by the National Institutes of Health. The government has certain rights in the invention.

### INCORPORATION BY REFERENCE OF SEQUENCE LISTING

This application contains a Sequence Listing which has been submitted electronically in ST26 format and is hereby incorporated by reference in its entirety. Said ST26 file, created on Apr. 3, 2024, is named “800133US1.xml” and is 45,380 bytes in size.

### BACKGROUND

Influenza is a major respiratory disease in some mammals including horses and is responsible for substantial morbidity and economic losses each year. Influenza virus infections can cause severe systemic disease in some avian species, leading to death. The segmented nature of the influenza virus genome allows for reassortment of segments during virus replication in cells infected with two or more influenza viruses. The reassortment of segments, combined with genetic mutation and drift, can give rise to a myriad of divergent strains of influenza virus over time. The new strains exhibit antigenic variation in their hemagglutinin (HA) and/or neuraminidase (NA) proteins, and in particular the gene coding for the HA protein has a high rate of variability.

The predominant current practice for the prevention of flu is vaccination. As the influenza HA protein is the major target antigen for the protective immune responses of a host to the virus and is highly variable, the isolation of influenza virus and the identification and characterization of the HA antigen in viruses associated with recent outbreaks is important for vaccine production. Based on prevalence and prediction, a vaccine is designed to stimulate a protective immune response against the predominant and expected influenza virus strains (Park et al., 2004).

There are three general types of influenza viruses, Type A, Type B and Type C, which are defined by the absence of serological crossreactivity between their internal proteins. Influenza Type A viruses are further classified into subtypes based on antigenic and genetic differences of their glycoproteins, the HA and NA proteins. Most of all the known HA and NA subtypes (H1 to H16 and N1 to N9) have been isolated from aquatic birds, which are though to act as a natural reservoir for influenza, although the genetic material of H17N10 and H18N 11 was isolated from bats. The H1N1 virus caused a pandemic in 2009. The first vaccine candidates tested in 2009 did not grow to high titers, demonstrating the need to develop vaccine virus backbones that confer efficient replication to vaccine virus candidates.

During the last decades, influenza A viruses of the H1N1 and H3N2 subtypes together with viruses representing two lineages of influenza B viruses have circulated in humans, although one of the influenza B virus lineages has not been detected since the outbreak of the SARS-CoV-2 pandemic. Annually, about 3 to 5 million cases of severe illness and an estimated 290,000 to 650,000 respiratory deaths worldwide are attributed to influenza virus infections (Health, 2018; luliano et al., 2018). In addition to the disease burden, influenza is also a considerable economic burden to the society.

Various vaccines and antiviral drugs have been developed to reduce the burden of influenza virus infection. To date, vaccination continues to be one of the best prevention strategies to reduce the morbidity and mortality caused by human influenza A and B viruses. The emergence of mutations in the major antigenic epitopes of HA can render circulating viruses resistant to antibodies induced by an influenza virus infection or vaccination and therefore necessitates frequent updates of the vaccine strains. Most egg-propagated influenza A vaccines are based on a vaccine virus backbone comprised of six influenza viral RNA (vRNA) segments (e.g., all vRNA segments except for the HA and NA vRNAs) derived from A/Puerto Rico/8/34 (PR8, H1N1) virus, which confers efficient replication. This vaccine virus backbone is then combined with the HA and NA vRNAs of the vaccine strain recommended by the WHO strain selection committee for the respective influenza season. Once the desired HA and NA vRNAs have been combined with the vaccine virus backbone, the resulting egg-propagated vaccine viruses are amplified in embryonated chicken eggs, which remain the most widely used platform for human influenza vaccine production. Vaccine virus yield is an important parameter in the vaccine manufacturing process.

### SUMMARY

As described herein, influenza A/Puerto Rico/8/34 viruses were developed that replicate to high titers in cultured cells and/or embryonated chicken eggs. Mutations were identified that resulted in higher virus titers in cultured cells and/or embryonated chicken eggs, allowing more efficient influenza virus growth and more cost-effective vaccine production. Replication-enhancing residues include, but are not limited to, PB2 439H, PB1 577R, PB1 640V, M1 35R, PB1 62E, and/or PB1 624I, or combinations thereof.

Viruses with the replication-enhancing residues are useful for promoting replication of influenza A viruses to high titers in embryonated chicken eggs and therefore increase the manufacturing yield for influenza vaccine viruses having those residues. These mutations can be used in the backbone gene segments of influenza A for improved yield in embryonated chicken eggs. The relevant HA and NA gene segment sequences for the currently circulating influenza viruses can be added to these backbone gene segments.

Individual growth-enhancing residues in an influenza virus polypeptide may be combined with one or more other growth-enhancing residues in the same influenza virus polypeptide, or with one or more other growth-enhancing residues in other influenza virus polypeptide(s), as well with growth-enhancing nucleotides in viral non-coding regions, e.g., promoter sequences. For example, one or more growth-enhancing residues in a polymerase protein, for instance, 1, 2, 3, 4, 5, 6, 7 or more, growth-enhancing residues in PB2, 1, 2, 3, 4, 5, 6, 7 or more, e.g., up to 12, 13, 14 or 15, growth-enhancing residues in PB1, 1, 2, 3, or 4 or more growth-enhancing residues in PA, or 1, 2, 3, or 4 growth-enhancing residues in NP, 1, 2, 3, or 4 growth-enhancing residues in M, e.g., 1, 2, or 3 growth-enhancing residues in M1, 1, 2, or 3 growth-enhancing residues in NS1, or any combination of growth-enhancing residues or nucleotides in viral non-coding, e.g., promoter sequences, may be combined when preparing influenza virus, e.g., for a vaccine, to enhance viral titers. In embodiments, growth-enhancing nucleotides in viral promoter sequences may be introduced to a viral segment, or when present in a viral segment may be selected for inclusion in an influenza virus. In embodiments, growth-enhancing residues in HA and/or in NA may be introduced into, or when present in a HA or NA selected for inclusion in, a HA viral segment or a NA viral segment in an influenza virus. In embodiments, the one or more growth-enhancing residues may enhance viral growth by at least 1.2, 2, 2.8, 4, 3, 5, 6, 8, 10, 100, or 200-fold or more.

Mutations that increase the replicative ability of viruses in cell culture and/or embryonated chicken eggs are useful to amplify influenza viruses and to establish robust influenza vaccine platforms. Currently, most influenza vaccines are generated in embryonated chicken eggs. Influenza vaccines generated in MDCK cells are now approved for human use in the U.S. and in Europe, and influenza vaccines derived from Vero cells are approved for human use in Europe. In addition to mutations in the coding regions of the six internal viral segments, mutations in non-coding regions were observed to increase viral titers, including promoter mutations, for instance, C-to-U mutations at position 4 from the 3′ end of the PB2, PB1, and/or PA vRNA segments. The resulting sequences may be also codon-usage optimized, e.g., optimized for expression in mammalian cells such as canine cells or primate cells, or avian cells, e.g., chicken embryos. The mutations can be used in various combinations, with results influenced by the cell line (or egg) in use and the desired level of improvement in the replication of the virus.

In embodiments, the disclosure provides an isolated recombinant influenza virus having PA, PB1, PB2, NP, NS, and M viral segments from a first influenza vaccine virus isolate, a heterologous, recombinant or chimeric influenza virus NA viral segment, and a heterologous, recombinant or chimeric HA viral segment, wherein the PB1 viral segment encodes a PB1 polypeptide having a residue other than glycine (G) or alanine (A) at position 62, a residue other than lysine (K) at position 577, a residue other than leucine (L) or valine (V) at position 624, and/or a residue other than methionine (M) at position 640, wherein the PB2 segment encodes a PB2 polypeptide having a residue other than glutamine (Q) at position 439, or wherein the M segment encodes a M1 polypeptide having a residue other than K at position 35; or a combination thereof. In embodiments, the residue at position 62 in PB1 comprises glutamic acid (E), aspartic acid (D), K, arginine (R), or histidine (H). In embodiments, the residue at position 577 in PB1 comprises R, K, D, E or H. In embodiments, the residue at position 624 in PB1 comprises isoleucine (I), alanine (A), G, or threonine (T). In embodiments, the residue at position 640 in PB1 comprises V, I, A, G, or T. In embodiments, the residue at position 439 in PB2 comprises H, R, K, D, or E. In embodiments, the residue at position 35 in M1 comprises R, H, D or E. In embodiments, the PB2 viral segment further encodes a PB2 with a V at position 504. In embodiments, the PA viral segment encodes a PA with a K at position 401. In embodiments, the PB1 viral segment further encodes a PB1 having a leucine at position 40 and/or tryptophan (W) at position 180. In embodiments, the NP viral segment encodes a NP polypeptide having leucine at position 116. In embodiments, the NS viral segment encodes a NS1 polypeptide having a proline at position 30 and/or a lysine at position 118. In embodiments, at least one of the PA, PB1, PB2, NP, NS, and M viral segments has a C to U promoter mutation. In embodiments, the PB1, PB2 and/or PA viral segment(s) comprise a C4U promoter mutation. In embodiments, the NA viral segment and the HA viral segment are from the same influenza virus isolate. In embodiments, at least one of the PA, PB1, PB2, NP, NS, and M viral segments comprise: a PB1 with at least 95% amino acid sequence identity to the PB1 encoded by SEQ ID NO:2; a PB2 with at least 95% amino acid sequence identity to the PB2 encoded by SEQ ID NO:3; a PA with at least 95% amino acid sequence identity to the PA encoded by SEQ ID NO:1; a NP with at least 95% amino acid sequence identity to the NP encoded by SEQ ID NO:4; a M1 with at least 95% amino acid sequence identity to the M1 encoded by SEQ ID N0:5; or a NS1 or NS2 with at least 95% amino acid sequence identity to the NS1 or NS2 encoded by SEQ ID NO:6. In embodiments, at least one of the PA, PB1, PB2, NP, NS, and M viral segments comprise: a PB1 with at least 95% amino acid sequence identity to the PB1 encoded by SEQ ID NO:10; a PB2 with at least 95% amino acid sequence identity to the PB2 encoded by SEQ ID NO:11; a PA with at least 95% amino acid sequence identity to the PA encoded by SEQ ID NO: 12, a NP with at least 95% amino acid sequence identity to the NP encoded by SEQ ID NO: 13, a M1 with at least 95% amino acid sequence identity to the M1 encoded by SEQ ID NO: 14; or a NS1 or NS2 with at least 95% amino acid sequence identity to the NS1 or NS2 encoded by SEQ ID NO:15.

Further provided is a vaccine having an effective amount of the isolated recombinant virus and a pharmaceutically acceptable carrier. In embodiments, the virus comprises a PB2 segment with a conservative substitution for 1504; a PB1 segment with a conservative substitution for M40 and a non-conservative substitution for G180; a PA segment with a conservative substitution for R401; a NP segment with a conservative substitution for 1116; and a NS segment with a conservative substitution for A30 or R118.

Also provided is a method to prepare influenza virus. The method includes contacting a cell with: a vector for vRNA or cRNA production comprising a promoter operably linked to an influenza virus PA DNA linked to a transcription termination sequence, a vector for vRNA or cRNA production comprising a promoter operably linked to an influenza virus PB1 DNA linked to a transcription termination sequence, a vector for vRNA or cRNA production comprising a promoter operably linked to an influenza virus PB2 DNA linked to a transcription termination sequence, a vector for vRNA or cRNA production comprising a promoter operably linked to an influenza virus HA DNA linked to a transcription termination sequence, a vector for vRNA or cRNA production comprising a promoter operably linked to an influenza virus NP DNA linked to a transcription termination sequence, a vector for vRNA or cRNA production comprising a promoter operably linked to an influenza virus NA DNA linked to a transcription termination sequence, a vector for vRNA or cRNA production comprising a promoter operably linked to an influenza virus M DNA linked to a transcription termination sequence, and a vector for vRNA or cRNA production comprising a promoter operably linked to an influenza virus NS DNA linked to a transcription termination sequence, wherein the PB1, PB2, PA, NP, NS, and M DNAs in the vectors for vRNA or cRNA production are from one or more influenza vaccine virus isolates, wherein the NA DNA in the vector for vRNA or cRNA production of NA has sequences for a heterologous, recombinant or chimeric NA, and wherein the HA DNA in the vector for vRNA or cRNA production of HA has sequences for a heterologous, recombinant or chimeric HA, wherein the PB1 DNA encodes a PB1 polypeptide having a residue other than glycine or alanine at position 62, a residue other than lysine at position 577, a residue other than leucine or valine at position 624, and/or a residue other than methionine at position 640, or wherein the PB2 DNA encodes a PB2 polypeptide having a residue other than glutamine at position 439, and/or wherein the M DNA encodes a M1 polypeptide having a residue other than lysine at position 35, or a combination thereof; and a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, or a vector for mRNA production comprising a promoter operably linked to a DNA segment encoding influenza virus NS2; in an amount effective to yield infectious influenza virus. In embodiments, the cell is an avian cell. In embodiments, the cell is a mammalian cell. In embodiments, the cell is a Vero cell, a human cell or a MDCK cell. In embodiments, the PB1, PB2, PA, NP, NS, and M DNAs in the vectors for vRNA or cRNA productions have a sequence that corresponds to one that encodes a polypeptide having at least 95% amino acid sequence identity to a corresponding polypeptide encoded by SEQ ID NOs:1-6 or 10-15. In embodiments, the method further comprises isolating the virus. In embodiments, at least one of PA, PB1, or PB2 viral segments has a C to U promoter mutation. Also provided is virus obtained by the method. In embodiments, the virus comprises PB2 having at least 95% amino acid sequence identity to a polypeptide encoded by SEQ ID NO:3 and having a valine at position 504 and/or a histidine at position 439, or a combination thereof; PB1 having at least 95% amino acid sequence identity to a polypeptide encoded by SEQ ID NO:2 and having a leucine at position 40, an alanine or isoleucine at position 54, a glutamic acid at position 62, glycine at position 112, histidine at position 247, valine at position 507, arginine at position 577, valine at position 640, isoleucine at position 624, alanine at position 644, or cysteine at position 713, or any combination thereof; or M1 having at least 95% amino acid sequence identity to a polypeptide encoded by SEQ ID NO:6 and having an arginine at position 35; or any combination thereof. In embodiments, the virus comprises a PB2 segment with a conservative substitution for 1504; a PB1 segment with a conservative substitution for M40 and a non-conservative substitution for G180; a PA segment with a conservative substitution for R401; a NP segment with a conservative substitution for 1116; and a NS segment with a conservative substitution for A30 or R118.

In embodiments, a vector is provided for vRNA or mRNA expression of a) influenza virus PB1 having at least 85% amino acid sequence identity to a polypeptide encoded by SEQ ID NO:2 and having a residue other than glycine or alanine at position 62, a residue other than lysine at position 577, a residue other than leucine or valine at position 624, and/or a residue other than methionine at position 640, or a combination thereof; b) influenza virus PB2 having at least 85% amino acid sequence identity to SEQ ID NO:3 and having a residue other than glutamine at position 439, or c) influenza virus M1 having at least 85% amino acid sequence identity to a M1 encoded by SEQ ID NO:6 and a residue other than lysine at position 35.

In embodiments, a method of immunizing an avian or a mammal is provided comprising: administering an effective amount of a composition comprising the virus or the vaccine to the avian or mammal. In embodiments, the mammal is a human. In embodiments, the composition further comprises at least one other virus. In embodiments, the composition comprises two to three other viruses. In embodiments, the viruses are influenza viruses that are genetically distinct from the virus of the disclosure. In embodiments, the composition is locally administered. In embodiments, the composition is intranasally administered. In embodiments, the composition is subcutaneously administered. In embodiments, the composition is intramuscularly administered.

The virus may have other specific residues at specified positions, e.g., 142N, 225C, 356R, or 550L in PA; has one or more of 112G (PB1-F2-R81G), 247H, 507V, or 644A in PB1; has one or more of 202L, 323L or 504V in PB2; has one or more of 74K, 112L, 116L, 417D, or 442A in NP; 97A and/or 100H in M1; and/or 55E and/or 140Q in NS1, or combinations thereof, or at least one of 202L and/or 323L in PB2, 247H in PB1 or 74K in NP and optionally at least one of 142N in PA1, 55K in NS1 or 97A and/or 100H in M1, e.g., 202L and/or 323L in PB2, has 247H in PB1, has 74K in NP, has 202L and/or 323L in PB2 and has 247H in PB1, or has 202L and/or 323L in PB2, has 247H in PB1, and has 74K in NP. For example, other specific residues at specified positions, include but are not limited to 401, 40L, 112G (PB1-F2-R81G), 180W, 247H, 507V, or 644A in PB1; which has 202L and/or 323L in PB2; which has 74K, 112L, 116L, 377N, 417D, or 422L in NP; which has 30P, 118K, 161T or 140Q in NS1; which has 142N, 225C, 356R, 401K, or 550L in PA; which has 401, 40L, 112G (PB1-F2-R81G), 180W, 247H, 507V, or 644A in PB1 and has 202L and/or 323L in PB2; which has 401, 40L, I12G (PB1-F2-R81G), 180W, 247H, 507V, or 644A in PB1, has 202L and/or 323L in PB2 and has 74K, 112L, 116L, 377N, 417D, or 422L in NP; which has 401, 40L, 112G (PB1-F2-R81G), 180W, 247H, 507V, or 644A in PB1, has 202L and/or 323L in PB2, has 74K, 112L, 116L, 377N, 417D, or 422L in NP, and has 30P, 118K, 161T or 140Q in NS1; which has 401, 40L, 112G (PB1-F2-R81G), 180W, 247H, 507V, or 644A in PB1, has 202L and/or 323L in PB2, has 74K, 112L, 116L, 377N, 417D, or 422L in NP, has 30P, 118K, 161T or 140Q in NS1, and has 142N, 225C, 356R, 401K, or 550L in PA; or which has 401, 40L, 112G, 180W, 247H, 507V, or 644A in PB1, has 202L and/or 323L in PB2, has 74K, 112L, 116L, 377N, 417D, or 422L in NP, has 30P, 118K, 161T or 140Q in NS1, and has 142N, 225C, 356R, 401K, or 550L in PA.

The disclosure provides isolated recombinant, e.g., reassortant, influenza viruses with selected amino acid residues at one or more specified positions in one or more viral segments for PA, PB1, PB2, NP, M (encoding M1 and M2 proteins), and/or NS (encoding NS1 and NS2 proteins), e.g., in selected amino acid residues at specified positions of PB1, PB2 and NS1; PA, PB1, PB2, NP and NS1; PB1, PB2, NP, M, and NS1; PA, PB1, PB2, NP and NS1; or PA, PB1, PB2, NP, M, and NS1, and including HA and NA genes/proteins of interest, e.g., from annual and pandemic strains, which viruses are produced more efficiently and cost-effectively via cell culture (in MDCK or Vero cells) or in embryonated chicken eggs. As used herein, a “viral segment” in a virus means an influenza vRNA sequence and a “viral segment” in a transcription cassette for production of a viral segment means a sequence that when introduced into a cell or appropriate cell-free system and transcribed, yields influenza vRNA or cRNA. In embodiments, the recombinant reassortant influenza virus has an amino acid residue at position 142 in PA that results in enhanced growth in cells including MDCK cells, Vero cells or eggs relative to a corresponding virus with, for instance, a lysine at position 142 in PA, i.e., the residue at position 142 in PA in the PA viral segment in the recombinant influenza virus is not lysine but is a residue that is correlated with enhanced replication in MDCK cells, Vero cells or eggs, as well as optionally selected amino acid residues at one or more specified positions in PB1, PB2, NP, M1 and/or NS1. In embodiments, the recombinant reassortant influenza virus has an amino acid residue at position 142 in PA that result in enhanced interaction with one or more host proteins in MDCK cells, Vero cells or eggs relative to a corresponding virus with, for instance, a lysine at position 142 in PA. In embodiments, the recombinant reassortant influenza virus has an asparagine or glutamine at position 142 in PA as well as optionally selected amino acid residues at one or more specified positions in PB1, PB2, NP, M1 and/or NS1. In embodiments, the recombinant reassortant influenza virus has an amino acid residue at position 247 in PB1 that results in enhanced growth in cells including MDCK cells, Vero cells or eggs relative to a corresponding virus with, for instance, a glutamine at position 247 in PB1, i.e., the residue at position 247 in PB1 in the PB1 viral segment in the recombinant influenza virus is not glutamine but is a residue that is correlated with enhanced replication in MDCK cells, Vero cells or eggs, as well as optionally selected amino acid residues at one or more specified positions PA, PB2, NP, M1 and/or NS1 which have are described herein. In embodiments, the recombinant reassortant influenza virus has an amino acid residue at position 247 in PB1 that results in enhanced interaction with one or more host proteins in MDCK cells, Vero cells or eggs relative to a corresponding virus with, for instance, a glutamine at position 247 in PB1. In embodiments, the recombinant reassortant influenza virus has a histidine, arginine or lysine at position 247 in PB1 as well as optionally selected amino acid residues at one or more specified positions PA, PB2, NP, M1 and/or NS1 which are described herein. In embodiments, the recombinant reassortant influenza virus has an amino acid residue at position 202 and/or position 323 in PB2 that results in enhanced growth in cells including MDCK cells, Vero cells or eggs relative to a corresponding virus with, for instance, a methionine at position 202 or a phenylalanine at position 323 in PB2, i.e., the residue at position 202 and/or 323 in PB2 in the PB2 viral segment in the recombinant influenza virus is not methionine or phenylalanine but is a residue that is correlated with enhanced replication in MDCK cells, Vero cells or eggs, as well as optionally selected amino acid residues at one or more specified positions PA, PB1, NP, M1 and/or NS which are described herein. In embodiments, the recombinant reassortant influenza virus has an amino acid residue at position 323 in PB2 that results in an altered cap binding interaction relative to a corresponding virus with, for instance, a phenylalanine at position 323 in PB2. In embodiments, the recombinant reassortant influenza virus has a leucine, alanine, threonine, valine, isoleucine, or glycine, at position 202 and/or position 323 in PB2 as well as optionally selected amino acid residues at one or more specified positions PA, PB1, NP, M1 and/or NS which are described herein. In embodiments, the recombinant reassortant influenza virus has an amino acid residue at position 74 in NP that results in enhanced growth in cells including MDCK cells, Vero cells or eggs relative to a corresponding virus with, for instance, an arginine at position 74 in NP, i.e., the residue at position 74 in NP in the NP viral segment in the recombinant influenza virus is not arginine but is a residue that is correlated with enhanced replication in MDCK cells, Vero cells or eggs, as well as optionally selected amino acid residues at one or more specified positions PA, PB1, PB2, M1 and/or NS which are described herein. In embodiments, the recombinant reassortant influenza virus has an amino acid residue at position 74 in NP that may alter folding, stability and/or interaction with other viral or host proteins relative to a corresponding virus with, for instance, an arginine at position 74 in NP. In embodiments, the recombinant reassortant influenza virus has a lysine or histidine at position 74 in NP as well as optionally selected amino acid residues at one or more specified positions PA, PB1, PB2, M1 and/or NS which are described herein. In embodiments, the recombinant reassortant influenza virus has an amino acid residue at position 97 and/or position 100 in M1 that results in enhanced growth in cells including MDCK cells, Vero cells or eggs relative to a corresponding virus with, for instance, a valine at position 97 or a tyrosine at position 100 in M1, i.e., the residue at position 97 and/or 100 in M1 in the M viral segment in the recombinant influenza virus is not valine or tyrosine, respectively, but is a residue that is correlated with enhanced replication in MDCK cells, Vero cells or eggs, as well as selected amino acid residues at one or more specified positions PA, PB1, PB2, NP and/or NS1 which are described herein. In embodiments, the recombinant reassortant influenza virus has an amino acid residue at position 97 in M1 that may alter dimerization relative to a corresponding virus with, for instance, a valine at position 97 in M1. In embodiments, the recombinant reassortant influenza virus has an amino acid residue at position 100 in M1 that may alter virus assembly relative to a corresponding virus with, for instance, a tyrosine at position 100 in M1. In embodiments, the recombinant reassortant influenza virus has a leucine, threonine, isoleucine, alanine, or glycine, at position 97 and/or a lysine, arginine, or histidine at position 100 in M1 as well as selected amino acid residues at one or more specified positions PA, PB1, PB2, NP and/or NS1 which are described herein. In embodiments, the recombinant reassortant influenza virus has an amino acid residue at position 55 in NS1 that results in enhanced growth in cells including MDCK cells, Vero cells or eggs relative to a corresponding virus with, for instance, a lysine at position 55 in NS1 as well as selected amino acid residues at one or more specified positions PA, PB1, PB2, NP and/or M1 which are described herein. In embodiments, the recombinant reassortant influenza virus has an asparagine, aspartic acid, glutamic acid or glutamine at position 55 in NS1 as well as selected amino acid residues at one or more specified positions PA, PB1, PB2, NP and/or M1 which are described herein. In embodiments, the disclosure provides an isolated recombinant reassortant influenza virus having six “internal” viral segments from a vaccine influenza virus with two or more of the selected amino acid residues at specified positions described herein, and a NA viral segment selected from a first influenza virus isolate, and a HA viral segment from the same isolate or a different isolate.

In embodiments, the influenza virus of the disclosure is a recombinant influenza virus having two or more of selected amino acid residues at specified positions in one or more viral segments for PA, PB1, PB2, NP, M1, and/or NS1, which can be employed with HA and NA genes of interest. In addition to the residues and position disclosed above, in embodiments, the recombinant reassortant influenza virus has an amino acid residue at position 142 in PA that results in enhanced growth in MDCK cells, Vero cells or eggs relative to a corresponding virus with, for instance, a lysine at position 142 in PA; an amino acid residue at position 247 in PB1 that results in enhanced growth in MDCK cells, Vero cells or eggs relative to a corresponding virus with, for instance, a glutamine at position 247 in PB1; an amino acid residue at position 202 and/or position 323 in PB2 that results in enhanced growth in MDCK cells, Vero cells or eggs relative to a corresponding virus with, for instance, a methionine at position 202 or a phenylalanine at position 323 in PB2; an amino acid residue at position 74 in NP that results in enhanced growth in MDCK cells, Vero cells or eggs relative to a corresponding virus with, for instance, a arginine at position 74 in NP; an amino acid residue at position 97 and/or position 100 in M1 that results in enhanced growth in MDCK cells, Vero cells or eggs relative to a corresponding virus with, for instance, a valine at position 97 or a tyrosine at position 100 in M1; or an amino acid residue at position 55 in NS1 that results in enhanced growth in MDCK cells, Vero cells or eggs relative to a corresponding virus with, for instance, a lysine at position 55 in NS1, or combinations thereof. In embodiments, the virus comprises PB2 having at least 95% amino acid sequence identity to a polypeptide encoded by SEQ ID NO:3 and having a valine at position 504 and/or a histidine at position 439, or a combination thereof; PB1 having at least 95% amino acid sequence identity to a polypeptide encoded by SEQ ID NO:2 and having a leucine at position 40, an alanine or isoleucine at position 54, a glutamic acid at position 62, glycine at position 112, histidine at position 247, valine at position 507, arginine at position 577, valine at position 640, isoleucine at position 624, alanine at position 644, or cysteine at position 713, or any combination thereof; or M1 having at least 95% amino acid sequence identity to a polypeptide encoded by SEQ ID NO:6 and having an arginine at position 35; or any combination thereof. In embodiments, the virus comprises a PB2 segment with a conservative substitution for 1504; a PB1 segment with a conservative substitution for M40 and a non-conservative substitution for G180; a PA segment with a conservative substitution for R401; a NP segment with a conservative substitution for 11 16; and a NS segment with a conservative substitution for A30 or R118.

In embodiments, the influenza virus of the disclosure is a recombinant influenza virus having two or more of selected amino acid residues at specified positions in one or more viral segments for PA, PB1, PB2, NP, M1, and/or NS1, which can be employed with HA and NA genes of interest. In addition to the residues and position disclosed above, in embodiments, the recombinant reassortant influenza virus has two or more of a lysine at position 142 in PA; a glutamine at position 247 in PB1; a leucine at position 202 and/or at position 323 in PB2; a lysine at position 74 in NP; an alanine at position 97 and an histidine at position 100 in M1; or a glutamic acid at position 55 in NS1.

The disclosure provides isolated recombinant, e.g., reassortant, influenza viruses with selected amino acid residues at one or more specified positions in one or more viral segments for PA, PB1, PB2, NP, M1, and/or NS1, e.g., in selected amino acid residues at specified positions PB1, PB2 and NS; PB1, PB2, NP and NS; PA, PB1, PB2, NP and NS; PB1, PB2, NP, M and NS, or PA, PB1, PB2, NP, M, and NS, that include one or more of the characteristic residues described herein. In addition to the residues and position disclosed above, the recombinant reassortant influenza virus can have an amino acid residue at position 105 and/or 401 in PA that result in enhanced growth in cells, e.g., MDCK cells, relative to a corresponding virus with, for instance, a phenylalanine or arginine at position 105 or 401, respectively, in PA. In addition to the residues and position disclosed above, the recombinant reassortant influenza virus can have an amino acid residue at position 40, 54, 59, 62, e.g., G62A, 63, 66 (F2), 73 (F2), 75, 76, 78, 79, 80, 112, 180, 261, 327, 361, 507, 621, 624, 644, 654, 667, 694, 695, 697, 699, 700, 701, 702, 705, 713, and/or 714 in PB1 that results in enhanced growth in cells, e.g., MDCK cells, relative to a corresponding virus with, for instance, a methionine, arginine, threonine, glycine, alanine, asparagine, lysine, glutamic acid, aspartic acid, glutamic acid, proline, serine, glutamic acid, glycine, serine, arginine, serine, methionine, glutamine, leucine, valine, asparagine, isoleucine, asparagine, leucine, glutamic acid, phenyalanine, phenylalanine, proline, serine, tyrosine, serine or methionine, at position 40, 54, 59, 62, 63, 66 (F2), 73 (F2), 75, 76, 78, 79, 80, 81(F2), 112, 180, 504, 507, 624, 644, 667, 694, 695, 697, 699, 700, 701, 702, 705, 713, or 714, respectively, in PB1. In addition to the residues and position disclosed above, in embodiments, the recombinant reassortant influenza virus has an amino acid residue at position 57, 58, 59, 61, 66, 202, 323, 368, 391, 504, 591, 677, 678, or 679 in PB2 that results in enhanced growth in cells, e.g., MDCK cells, relative to a corresponding virus with, for instance, an isoleucine, threonine, alanine, lysine, methionine, methionine, phenylalanine, arginine, glutamic acid, isoleucine, glutamine, glutamic acid, aspartic acid or phenylalanine, at position 57, 58, 59, 61, 66, 202, 323, 368, 391, 504, 591, 677, 678 or 679, respectively, in PB2. In addition to the residues and position disclosed above, in embodiments, the recombinant reassortant influenza virus has an amino acid residue at position 116, 224, 293, 371, 417, 422 or 442 in NP that results in enhanced growth in cells, e.g., MDCK cells, relative to a corresponding virus with, for instance, a leucine, asparagine, arginine, methionine, aspartic acid, arginine or threonine, at position 116, 224, 293, 371, 417, 422, or 442, respectively, in NP. In addition to the residues and position disclosed above, in embodiments, the recombinant reassortant influenza virus has an amino acid residue at position 90 in M1 that results in enhanced growth in cells relative to a corresponding virus with, for instance, a serine at position 90 in M1.

In addition to the residues and position disclosed above, in embodiments, the recombinant reassortant influenza virus has an amino acid residue at position 30, 49, 118, 140, 161, or 223 in NS1 that results in enhanced growth in MDCK cells relative to a corresponding virus with, for instance, a proline, alanine, arginine, glutamine, threonine, or glutamic acid, respectively, at position 30, 49, 140, 161 or 223, respectively, in NS1. In addition to the residues and position disclosed above, in embodiments, the recombinant influenza virus does not have a valine at residue 504 in PB2 and a leucine at residue 550 in PA. In embodiments, the recombinant reassortant influenza virus has an amino acid residue at position 136, 162, 179, 182, 184, 252, 449, and/or 476 (or any combination thereof) in HA (the numbers refer to the amino acid positions in H3 HA after removal of the N-terminal signal peptide) that results in enhanced growth in cells relative to a corresponding virus with, for instance, glutamic acid, lysine, glutamine, leucine, valine, phenylalanine, lysine or methionine at position 136, 162, 179, 182, 184, 252, 449, or 476 in HA. In addition to the residues and position disclosed above, in embodiments, the recombinant influenza virus has an amino acid residue at position 55 or 265, or both, in NA (the numbers refer to the amino acid positions in N1 NA) that results in enhanced growth in cells relative to a corresponding virus with, for instance, leucine or alanine at position 55 or 265, respectively, in NA.

In embodiments, the influenza virus of the disclosure is a recombinant influenza virus having a particular amino acid residue at specified positions in one, two, three or more of PA, PB1, PB2, NP, M1 and/or NS1 and having an amino acid sequence with at least 80%, e.g., 90%, 92%, 95%, 97%, 98%, or 99%, including any integer between 80 and 99, contiguous amino acid sequence identity to a corresponding polypeptide encoded by one of SEQ ID Nos. 1-6 or 10-15. The residue other than the specified residue may be conservative substitution. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine and tryptophan; a group of amino acids having basic side chains is lysine, arginine and histidine; and a group of amino acids having sulfur-containing side chain is cysteine and methionine. In embodiments, conservative amino acid substitution groups are: threonine-valine-leucine-isoleucine-alanine; phenylalanine-tyrosine; lysine-arginine; alanine-valine; glutamic-aspartic; and asparagine-glutamine.

In embodiments, the influenza virus of the disclosure is a recombinant influenza virus having a particular amino acid residue at specified positions in PA, PB1, PB2, NP, M1 and/or NS1 and an amino acid sequence with at least 80%, e.g., 90%, 92%, 95%, 97%, 98%, or 99%, including any integer between 80 and 99, contiguous amino acid sequence identity to a corresponding polypeptide encoded by one of SEQ ID Nos. 1-6 or 10-15, e.g., a PB2 segment with a conservative substitution for 1504; a PB1 segment with a conservative substitution for M40 and a non-conservative substitution for G180; a PA segment with a conservative substitution for R401; a NP segment with a conservative substitution for I116; a NS viral segment with a conservative substitution for A30 or R118.

Also included are any combination of the selected amino acid residues at specified positions described herein.

Viral segments for of PA, PB1, PB2, NP, M and/or NS that have the residues at the specified positions may be combined with a viral segment for HA, e.g., H1, H2, H3, H4, H5, H6, H7, H8, H9, H10, H11, H12, H13, H14, H15, H16, or H17 and a viral segment for NA, e.g., N1, N2, N3, N4, N5, N6, N7, N8, N9, or N10, and any combination of HA and NA, to provide the reassortant vaccine viruses of the disclosure. In embodiments, the HA is H1, H5 or H7. In one embodiment the NA is N1 or N9. In embodiments, the HA viral segment in the reassortant virus is heterologous to the viral segments for PA, PB1, PB2, NP, M and NS. In embodiments, the NA viral segment in the reassortant virus is heterologous to the viral segments for PA, PB1, PB2, NP, M and NS. In embodiments, the HA viral segment in the reassortant virus has viral segments for PA, PB1, PB2, NP, M and NS from one influenza virus isolate or strain (“parent”), or a variant thereof, e.g., one with viral segments encoding influenza virus proteins with at least 95%, 96%, 97%, 98%, 99%, or 99.5% amino acid sequence identity, or having 1, 2, 5, 10, or 20 substitutions relative, to sequences in a parent influenza virus isolate or strain. In embodiments, the parent strain has viral segments with sequences corresponding to SEQ ID Nos. 1-6 or 10-15. In embodiments, the HA viral segment in the reassortant virus is a chimeric HA viral segment, e.g., a chimera of heterologous HA ectodomain sequences linked to HA signal peptide sequences and/or HA transmembrane domain sequences from the HA viral segment of the parent isolate or strain, or variant thereof. In embodiments, the NA viral segment in the isolated recombinant virus is a chimeric NA viral segment e.g., a chimera of heterologous NA ectodomain sequences linked to NA transmembrane domain sequences from the NA viral segment of the parent isolate or strain, or variant thereof, and/or stalk sequences from the parent isolate or strain, or variant thereof. In embodiments, the NA viral segment in the isolated recombinant virus is a chimeric NA viral segment e.g., a chimera of heterologous NA ectodomain sequences linked to NA transmembrane domain sequences from the NA viral segment of the parent isolate or strain, or variant thereof, and/or stalk sequences from a second isolate or strain, or variant thereof. In embodiments, the isolated recombinant virus has a heterologous HA viral segment, a heterologous NA viral segment, a chimeric HA viral segment, a chimeric NA viral segment, or any combination thereof. The nucleic acid sequences employed to prepare vRNA may be ones that introduce the residues at the specified positions via recombinant methodology or may be selected as having the residues at the specified positions.

A/Puerto Rico/8/34 (H1N1), “PR8,” virus serves as the genetic backbone for generation of inactivated influenza vaccines. Occasionally, vaccine strains based on PR8 backbone replicate to relatively low titers in eggs and cell culture resulting in delayed vaccine production and vaccine shortage. To determine if high yield vaccine strain backbones for propagation in MDCK cells, chicken eggs and Vero cells can be prepared to supply the demand of seasonal flu and highly pathogenic pandemic viruses, various mutagenesis strategies were employed. For example, PR8 backbone random mutant libraries were screened for high replicative mutants, e.g., by introducing random mutations to internal PR8 genes by error prone PCR, introducing mutations that confer high replication and high polymerase activity, and optimizing PR8 internal gene via codon bias. In another approach, the HA gene was optimized to increase virus replication and HA content, e.g., by optimizing the HA promoter to generate a strong promoter, optimizing the HA noncoding region, and/or optimizing the HA signal peptide.

Thus, for vaccine viruses that are to be grown or passaged in cells in culture, e.g., MDCK or Vero cells or eggs, selection of sequences with, or replacement of, the disclosed residues at the specified positions in one or more of PA, PB1, PB2, NP, M1 and/or NS1, that confer enhanced growth of the virus in cultured cells when employed with HA and NA sequences of interest, can result in significantly higher viral titers. Thus, the disclosure provides a method to select for influenza viruses with enhanced replication in cell culture. The method includes providing cells suitable for influenza vaccine production; serially culturing one or more influenza virus isolates in the cells; and isolating serially cultured virus with enhanced growth relative to the one or more isolates prior to serial culture. In embodiments, the cells are canine or primate, e.g., human or monkey, cells.

In embodiments, the influenza virus of the disclosure is a recombinant influenza virus having two or more of selected amino acid residues at specified positions in one or more of PA, PB1, PB2, NP, M1, and/or NS1, which can be employed with HA and NA genes of interest. In addition to the residues and position disclosed above, in embodiments, the recombinant reassortant influenza virus has an asparagine or glutamine at position 142 in PA, a cysteine at position 225, an arginine or histidine at position 356 in PA, or a leucine, valine, threonine, or glycine at position 550 in PA; a histidine, arginine or lysine at position 247 in PB1, a valine, leucine, isoleucine, threonine, alanine or glycine at position 507 in PB1 and/or an alanine, glycine, leucine or isoleucine at position 644 in PB1; a leucine, alanine, valine, isoleucine, glycine, or threonine at position 202 and/or position 323 in PB2, or a valine, leucine, glycine, threonine, or alanine at position 504 in PB2; a lysine or a histidine at position 74 in NP or a leucine, valine, glycine or alanine at position 112, 116 or 442 in NP; a leucine, isoleucine, alanine, glycine, or threonine, at position 97 and/or a lysine, arginine or histidine position 100 in M1; or an asparagine, aspartic acid, glutamic acid or glutamine at position 55 or glutamine or asparagine at position 140 in NS1.

The disclosure provides a plurality of influenza virus vectors of the disclosure, e.g., those useful to prepare reassortant viruses including 6:1:1 reassortants, 6:2 reassortants and 7:1 reassortants. A 6:1:1 reassortant within the scope of the present disclosure is an influenza virus with 6 internal viral segments from a vaccine virus, a NA viral segment from a different (second) viral isolate, and a HA viral segment from a third isolate; a 6:2 reassortant within the scope of the present disclosure is an influenza virus with 6 internal viral segments from a vaccine virus, and a NA viral segment and a HA viral segment from a different (second) viral isolate; and a 7:1 reassortant within the scope of the present disclosure is an influenza virus with 6 internal viral segments and a NA viral segment from a vaccine virus, and a HA viral segment from a different viral source than the vaccine virus, or an influenza virus with 6 internal viral segments and a HA viral segment from the vaccine virus, and a NA viral segment is from a different viral source than the vaccine virus.

In embodiments, the plurality includes vectors for vRNA or cRNA production selected from a vector comprising a promoter operably linked to an influenza virus PA DNA linked to a transcription termination sequence, a vector comprising a promoter operably linked to an influenza virus PB1 DNA linked to a transcription termination sequence, a vector comprising a promoter operably linked to an influenza virus PB2 DNA linked to a transcription termination sequence, a vector comprising a promoter operably linked to an influenza virus HA DNA linked to a transcription termination sequence, a vector comprising a promoter operably linked to an influenza virus NP DNA linked to a transcription termination sequence, a vector comprising a promoter operably linked to an influenza virus NA DNA linked to a transcription termination sequence, a vector comprising a promoter operably linked to an influenza virus M DNA linked to a transcription termination sequence, and a vector comprising a operably linked to an influenza virus NS DNA linked to a transcription termination sequence. In embodiments, the DNAs for vRNA or cRNA production of PB1, PB2, PA, NP, M, and NS, have sequences from an influenza virus that replicates to high titers in cultured mammalian cells such as MDCK cells, Vero cells or PER.C6® cells and also optionally embryonated eggs, and/or from a vaccine virus, e.g., one that does not cause significant disease in humans. The DNA for vRNA or cRNA production of NA may be from any NA, e.g., any of N1-N11, and the DNA for vRNA or cRNA production of HA may be from any HA, e.g., H1-H18. In embodiments, the DNAs for vRNA or cRNA production may be for an influenza B or C virus. The DNAs for vRNA or cRNA production of NA and HA may be from different strains or isolates (6:1:1 reassortants) or from the same strain or isolate (6:2 reassortants), or the NA may be from the same strain or isolate as that for the internal genes (7:1 reassortant). The plurality also includes vectors for mRNA production selected from a vector encoding influenza virus PA, a vector encoding influenza virus PB1, a vector encoding influenza virus PB2, and a vector encoding influenza virus NP, and optionally one or more vectors encoding NP, NS, M, e.g., M1 and M2, HA or NA. The vectors encoding viral proteins may further include a transcription termination sequence.

Viruses that may provide the internal genes for reassortants within the scope of the disclosure include viruses that have high titers in MDCK cells, e.g., titers of at least about 105 PFU/mL, e.g., at least 10⁶ PFU/mL, 10⁷ PFU/mL or 10⁸ PFU/mL; high titers in embryonated eggs, e.g., titers of at least about 10⁷ EID₅₀/mL, e.g., at least 10⁸ EID₅₀/mL, 10⁹ EID₅₀/mL or 10¹⁰ EID₅₀/mL; high titers in cells such as MDCK cells, e.g., titers of at least about 10⁷ PFU/mL, e.g., at least 10⁸ PFU/mL, or high titers in two of more of those host cells.

In embodiments, the titers of the reassortant viruses of the disclosure in cells such as MDCK cells or Vero cells may be over 1 log, 2 logs, 3 logs, or greater, than titers of the corresponding virus without particular residues at the specified positions.

Other reassortants with internal genes from other PR8 isolates or vaccine viruses may be employed in recombinant reassortant viruses of the disclosure. In particular, 5:1:2 reassortants having UW-PR8 PB1, PB2, PA, NP, and M (“5”) and PR8(Cam) NS (“1”); 6:1:1 reassortants having UW-PR8 NA, PB1, PB2, PA, NP, and M (“6”) and PR8(Cam) NS (“1”); and 7:1 reassortants having UW-PR8 PB1, PB2, PA, NP, M, NA, and NS (“7”) may be employed.

In embodiments, the DNAs for the internal genes for PB1, PB2, PA, NP, M, and NS encode proteins with substantially the same activity as a corresponding polypeptide encoded by one of SEQ ID NOs:1-6 or 10-15. As used herein, “substantially the same activity” includes an activity that is about 0.1%, 1%, 10%, 30%, 50%, 90%, e.g., up to 100% or more, or detectable protein level that is about 80%, 90% or more, the activity or protein level, respectively, of the corresponding full-length polypeptide. In embodiments, the nucleic acid a sequence encoding a polypeptide which is substantially the same as, e.g., having at least 80%, e.g., 90%, 92%, 95%, 97%, 98%, or 99%, including any integer between 80 and 99, contiguous amino acid sequence identity to, a polypeptide encoded by one of SEQ ID NOs:1-6 or 10-15. In embodiments, the isolated and/or purified nucleic acid molecule comprises a nucleotide sequence which is substantially the same as, e.g., having at least 50%, e.g., 60%, 70%, 80% or 90%, including any integer between 50 and 100, or more contiguous nucleic acid sequence identity to one of SEQ ID NOs:1-6 or 10-15 and, in embodiments, also encodes a polypeptide having at least 80%, e.g., 90%, 92%, 95%, 97%, 98%, or 99%, including any integer between 80 and 99, contiguous amino acid sequence identity to a polypeptide encoded by one of SEQ ID NOs:1-6 or 10-15. In embodiments, the influenza virus polypeptide has one or more, for instance, 2, 5, 10, 15, 20 or more, conservative amino acids substitutions, e.g., conservative substitutions of up to 10% or 20% of 2, 5, 10, 15, 20 or more, of a combination of conservative and non-conservative amino acids substitutions, e.g., conservative substitutions of up to 10% or 20% of the residues, or relative to a polypeptide encoded by one of SEQ IS NOs:1-6 or 10-15, and has a characteristic residue in two or more of PA, PB1, PB2, NP, M1, and/or NS1 the residues, relative to a polypeptide encoded by one of SEQ ID NOs:1-6 or 10-15, and has a characteristic residue in two or more of the viral segments for PA, PB1, PB2, NP, M1, and/or NS1, e.g., the influenza virus polypeptide has one or more, for instance, 2, 3, 4, 5, 6, 7 or 8 conservative and/or nonconservative amino acid substitutions, relative to a polypeptide encoded by one of SEQ ID NOs:1-6 or 10-15, e.g., those in virus isolates in Table 1.

The disclosure thus includes the use of isolated and purified vectors or plasmids, which express or encode influenza virus proteins, or express or encode influenza vRNA, both native and recombinant vRNA. The vectors comprise influenza cDNA, e.g., influenza A (e.g., any influenza A gene including any of the 16 HA or 9 NA subtypes), B or C DNA (see Fields Virology (Fields et al. (eds.), Lippincott, Williams and Wickens (2006), which is specifically incorporated by reference herein). Any suitable promoter or transcription termination sequence may be employed to express a protein or peptide, e.g., a viral protein or peptide, a protein or peptide of a nonviral pathogen, or a therapeutic protein or peptide.

A composition or plurality of vectors of the disclosure may also comprise a heterologous gene or open reading frame of interest, e.g., a foreign gene encoding an immunogenic peptide or protein useful as a vaccine or in gene replacement, for instance, may encode an epitope useful in a cancer therapy or vaccine, or a peptide or polypeptide useful in gene therapy. When preparing virus, the vector or plasmid comprising the gene or cDNA of interest may substitute for a vector or plasmid for an influenza viral gene or may be in addition to vectors or plasmids for all influenza viral genes. Thus, another embodiment of the disclosure comprises a composition or plurality of vectors as described above in which one of the vectors is replaced with, or further comprises, 5′ influenza virus sequences optionally including 5′ influenza virus coding sequences or a portion thereof, linked to a desired nucleic acid sequence, e.g., a desired cDNA, linked to 3′ influenza virus sequences optionally including 3′ influenza virus coding sequences or a portion thereof. In embodiments, the desired nucleic acid sequence such as a cDNA is in an antisense (antigenomic) orientation. The introduction of such a vector in conjunction with the other vectors described above to a host cell permissive for influenza virus replication results in recombinant virus comprising vRNA corresponding to the heterologous sequences of the vector.

The promoter in a vector for vRNA or cRNA production may be a RNA polymerase I promoter, a RNA polymerase I1 promoter, a RNA polymerase III promoter, a T7 promoter, or a T3 promoter, and optionally the vector comprises a transcription termination sequence such as a RNA polymerase I transcription termination sequence, a RNA polymerase II transcription termination sequence, a RNA polymerase III transcription termination sequence, or a ribozyme. Ribozymes within the scope of the disclosure include, but are not limited to, tetrahymena ribozymes, RNase P, hammerhead ribozymes, hairpin ribozymes, hepatitis ribozyme, as well as synthetic ribozymes. In embodiments, the RNA polymerase I promoter is a human RNA polymerase I promoter.

The promoter or transcription termination sequence in a vRNA or virus protein expression vector may be the same or different relative to the promoter or any other vector. In embodiments, the vector or plasmid which expresses influenza vRNA comprises a promoter suitable for expression in at least one particular host cell, e.g., avian or mammalian host cells such as canine, feline, equine, bovine, ovine, or primate cells including human cells, or for expression in more than one host.

In embodiments, at least one vector for vRNA comprises a RNA polymerase II promoter linked to a ribozyme sequence linked to viral coding sequences linked to another ribozyme sequences, optionally linked to a RNA polymerase II transcription termination sequence. In embodiments, at least 2, e.g., 3, 4, 5, 6, 7 or 8, vectors for vRNA or cRNA production comprise a RNA polymerase II promoter, a first ribozyme sequence, which is 5′ to a sequence corresponding to viral sequences including viral coding sequences, which is 5′ to a second ribozyme sequence, which is 5′ to a transcription termination sequence. Each RNA polymerase II promoter in each vRNA vector may be the same or different as the RNA polymerase II promoter in any other vRNA vector. Similarly, each ribozyme sequence in each vRNA vector may be the same or different as the ribozyme sequences in any other vRNA vector. In embodiments, the ribozyme sequences in a single vector are not the same.

In embodiments, the disclosure provides a plurality of influenza virus vectors for a reassortant, comprising a vector for vRNA or cRNA production comprising a promoter operably linked to an influenza virus PA DNA linked to a transcription termination sequence, a vector for vRNA or cRNA production comprising a promoter operably linked to an influenza virus PB1 DNA linked to a transcription termination sequence, a vector for vRNA or cRNA production comprising a promoter operably linked to an influenza virus PB2 DNA linked to a transcription termination sequence, a vector for vRNA or cRNA production comprising a promoter operably linked to an influenza virus HA DNA linked to a transcription termination sequence, a vector for vRNA or cRNA production comprising a promoter operably linked to an influenza virus NP DNA linked to a transcription termination sequence, a vector for vRNA or cRNA production comprising a promoter operably linked to an influenza virus NA DNA linked to a transcription termination sequence, a vector for vRNA or cRNA production comprising a promoter operably linked to an influenza virus M DNA linked to a transcription termination sequence, and a vector for vRNA or cRNA production comprising a promoter operably linked to an influenza virus NS cDNA linked to a transcription termination sequence, wherein the DNAs for PB1, PB2, PA, NP, NS, and M are from one or more influenza vaccine seed viruses and contain two or more of the characteristic residues at the specified position(s); and a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, or a vector for mRNA production comprising a promoter operably linked to a DNA segment encoding influenza virus NS2. In embodiments, at least one vector comprises sequences corresponding to those encoding PB1, PB2, PA, NP, M, or NS, or a portion thereof, having substantially the same activity as a corresponding polypeptide encoded by one of SEQ ID NOs:1-6 or 10-15, e.g., a sequence encoding a polypeptide with at least 80%, e.g., 85%, 90%, 92%, 95%, 98%, 99% or 100%, including any integer between 80 and 100, amino acid identity to a polypeptide encoded by one of SEQ ID NOs:1-6 or 10-15. Optionally, two vectors may be employed in place of the vector comprising a promoter operably linked to an influenza virus M cDNA linked to a transcription termination sequence, e.g., a vector comprising a promoter operably linked to an influenza virus M1 cDNA linked to a transcription termination sequence and a vector comprising a promoter operably linked to an influenza virus M2 cDNA linked to a transcription termination sequence.

A plurality of the vectors of the disclosure may be physically linked or each vector may be present on an individual plasmid or other, e.g., linear, nucleic acid delivery vehicle. In embodiments, each vRNA or cRNA production vector is on a separate plasmid. In embodiments, each mRNA production vector is on a separate plasmid.

The disclosure also provides a method to prepare influenza virus. The method comprises contacting a cell with a plurality of the vectors of the disclosure, e.g., sequentially or simultaneously, in an amount effective to yield infectious influenza virus. The disclosure also includes isolating virus from a cell contacted with the plurality of vectors. Thus, the disclosure further provides isolated virus, as well as a host cell contacted with the plurality of vectors or virus of the disclosure. In another embodiment, the disclosure includes contacting the cell with one or more vectors, either vRNA or protein production vectors, prior to other vectors, either vRNA or protein production vectors. In embodiments, the promoter for vRNA vectors employed in the method is a RNA polymerase I promoter, a RNA polymerase II promoter, a RNA polymerase III promoter, a T3 promoter or a T7 promoter. In embodiments, the RNA polymerase I promoter is a human RNA polymerase I promoter. In embodiments, each vRNA vector employed in the method is on a separate plasmid. In embodiments, the vRNA vectors employed in the method are on one plasmid or on two or three different plasmids. In embodiments, each mRNA vector employed in the method is on a separate plasmid. In embodiments, the mRNA vectors for PA, PB1, PB2 and NP employed in the method are on one plasmid or on two or three different plasmids.

In embodiments, the disclosure provides a method to select for influenza viruses with enhanced replication in cell culture. The method includes providing cells suitable for influenza vaccine production; serially culturing one or more influenza virus isolates in the cells; and isolating serially cultured virus with enhanced growth relative to the one or more isolates prior to serial culture. In embodiments, the cells are rodent or primate cells.

The methods of producing virus described herein, which do not require helper virus infection, are useful in viral mutagenesis studies, and in the production of vaccines (e.g., for AIDS, influenza, hepatitis B, hepatitis C, rhinovirus, filoviruses, malaria, herpes, and foot and mouth disease) and gene therapy vectors (e.g., for cancer, AIDS, adenosine deaminase, muscular dystrophy, ornithine transcarbamylase deficiency and central nervous system tumors). Thus, a virus for use in medical therapy (e.g., for a vaccine or gene therapy) is provided.

The disclosure also provides isolated viral polypeptides, and methods of preparing and using recombinant virus of the disclosure. The methods include administering to a host organism, e.g., a mammal, an effective amount of the influenza virus of the disclosure, e.g., an inactivated virus preparation, optionally in combination with an adjuvant and/or a carrier, e.g., in an amount effective to prevent or ameliorate infection of an animal such as a mammal by that virus or an antigenically closely related virus. In embodiments, the virus is administered intramuscularly while in another embodiment, the virus is administered intranasally. In some dosing protocols, all doses may be administered intramuscularly or intranasally, while in others a combination of intramuscular and intranasal administration is employed. The vaccine may further contain other isolates of influenza virus including recombinant influenza virus, other pathogen(s), additional biological agents or microbial components, e.g., to form a multivalent vaccine. In embodiments, intranasal vaccination, for instance containing with inactivated influenza virus, and a mucosal adjuvant may induce virus-specific IgA and neutralizing antibody in the nasopharynx as well as serum IgG.

The influenza virus of the disclosure may be employed with other anti-virals, e.g., amantadine, rimantadine, and/or neuraminidase inhibitors, e.g., may be administered separately in conjunction with those anti-virals, for instance, administered before, during and/or after.

### BRIEF DESCRIPTION OF THE FIGURES

The drawings illustrate generally, by way of example, but not by way of limitation, various embodiments discussed herein.

FIG. 1. Recombinant viruses generated with different PR8 backbone mutations.

FIG. 2. Overview of generation of viruses with enhanced growth in MDCK cells and Vero cells.

FIG. 3. Example high yield substitutions (relative to PR8 (UW)).

FIG. 4. Growth kinetics and HA titers of reassortant viruses possessing one or several vRNAs of PR8-HY virus.

FIG. 5. Viral polymerase activity in mini-replicon assays in 293T, MDCK, Vero, and DF1 cells. The PB2, PB1, PA, and NP proteins were derived from UW-PR8 wild-type (WT) virus or from the high-yield PR8-HY (HY) variant.

FIG. 6. Flow chart summarizing the selection and testing of the enhanced high-yield vaccine backbone.

FIG. 7. HA and virus titers in embryonated chicken eggs of two high-yield vaccine candidates. Viruses expressing the Singapore HA and NA proteins with the genetic backbone of the parental PR8-HY or the enhanced S12- and S13-HY candidates were inoculated into embryonated chicken eggs (2×10W PFU/egg) and HA and virus titers were measured 48 hours later. Shown are the averages of three independently generated virus stocks. The titers were compared by one-way ANOVA, followed by Tukey's post-hoc test. * p<0.05.

FIG. 8. Growth curves in embryonated chicken eggs of vaccine viruses and recombinant viruses with different genetic backbones. The HA and NA genes of egg-grown Singapore (a,b), Switzerland (c,d), Kansas (e,f) and Michigan (g,h) vaccine viruses were combined with the genetic backbone of parental PR8-HY or enhanced S12-HY. For comparison, we also tested authentic egg-grown Singapore (IVR-186) (a, b), Switzerland (NIB-112) (c,d), Kansas (NYMC X-327) (e,f) and Michigan (NYMC-X275) (g,h) vaccine viruses from NIBSC. 10-day-old embryonated chicken eggs were infected with 2×10² PFU of virus. The allantoic fluids of three eggs each were harvested at the indicated time points. HA and virus titers were determined by HA and plaque assays in MDCK cells, respectively. The titers were compared by two-way ANOVA analysis. *p<0.05; **p<0.01; ***p<0.001. Red and blue asterisks indicate the comparison of the ‘red’ or ‘blue’ virus with vaccine virus, respectively. Green asterisks indicate the comparison of S12-HY and PR8-HY backbone viruses.

FIG. 9. Evaluation of total viral protein and HA content of vaccine viruses and recombinant viruses with different genetic backbones. Total protein content of virus concentrate was determined by utilizing the Pierce BCA protein assay kit (Thermo Scientific) according to the manufacturer's instructions. Viral protein was deglycosylated by use of PNGase F (New England Biolabs) and then run the SDS-PAGE electrophoresis (4a). The HA contents were calculated according to the amounts of total viral protein (4b) and the amounts of HA (4c). The HA contents are expressed in mg per 100 eggs. Asterisks show a significant difference between groups. The values presented are the average of three independent values±s.d. Statistical significance was determined by using Student's t test. *p<0.05; **p<0.01; ***p<0.001; ***p<0.0001.

FIG. 10. Summary of mutant virus libraries and their consecutive passages in embryonated chicken eggs. All eight virus libraries were passaged twice in embryonated chicken eggs. Aliquots of the virus libraries were combined, resulting in a “mixed library”. The individual libraries and mixed library were passaged thirteen and fifteen more times in embryonated chicken eggs, respectively, followed by plaque assays. In total, 216 plaques were picked, amplified in eggs, and their HA titers were measured. The candidates with the highest HA titers were re-amplified in eggs, and viruses with increased HA titers were characterized in more detail.

FIG. 11. Nucleotide and amino acid sequence for PR8(Cambridge) genes (SEQ ID NOs:10-21).

### DETAILED DESCRIPTION

Compositions and methods are described herein for making and using influenza viruses that replicate to high titers in cultured cells and/or embryonated chicken eggs. The influenza viruses comprise mutations that result in more efficient influenza virus growth and more cost-effective vaccine production. In embodiments, an influenza A/Puerto Rico/8/34 virus strain can comprise replication-enhancing residues that include, but are not limited to PB2 439H, PB1 577R, PB1 640V, M1 35R, PB1 62E, and/or PB1 624I, or any combination thereof. The relevant HA and NA gene segment sequences for the currently circulating influenza viruses can be added to the backbone gene segments of influenza A containing these replication-enhancing residues for improved virus yield.

Vaccination is an effective way to prevent influenza virus infections. Influenza A and B virus vaccine backbones can be prepared that increased the yield of several vaccine viruses in, e.g., embryonated eggs, Madin-Darby canine kidney (MDCK) and/or African green monkey kidney (Vero) cells. Embryonated chicken eggs are the most frequently used platform for influenza vaccine manufacturing. To further increase the viral titers in embryonated chicken eggs, as disclosed herein, random mutations were introduced into the ‘internal genes’ (i.e., all influenza viral genes except those encoding the hemagglutinin and neuraminidase proteins) of the influenza A virus high-yield virus backbone. The randomly mutated viruses were sequentially passaged in embryonated chicken eggs to select variants with increased replicative ability. Candidates were identified which conferred higher influenza virus growth than the high-yield parental virus backbone. The enhanced virus backbone can be used to improve the titers of seasonal influenza vaccine viruses.

### Definitions

As used herein, the term “isolated” refers to in vitro preparation and/or isolation of a nucleic acid molecule, e.g., vector or plasmid, peptide or polypeptide (protein), or virus of the disclosure, so that it is not associated with in vivo substances, or is substantially purified from in vitro substances. An isolated virus preparation is generally obtained by in vitro culture and propagation, and/or via passage in eggs, and is substantially free from other infectious agents.

As used herein, “substantially purified” means the object species is the predominant species, e.g., on a molar basis it is more abundant than any other individual species in a composition, and preferably is at least about 80% of the species present, and optionally 90% or greater, e.g., 95%, 98%, 99% or more, of the species present in the composition.

As used herein, “substantially free” means below the level of detection for a particular infectious agent using standard detection methods for that agent.

A “recombinant” virus is one which has been manipulated in vitro, e.g., using recombinant DNA techniques, to introduce changes to the viral genome. Reassortant viruses can be prepared by recombinant or nonrecombinant techniques.

As used herein, the term “recombinant nucleic acid” or “recombinant DNA sequence or segment” refers to a nucleic acid, e.g., to DNA, that has been derived or isolated from a source, that may be subsequently chemically altered in vitro, so that its sequence is not naturally occurring, or corresponds to naturally occurring sequences that are not positioned as they would be positioned in the native genome. An example of DNA “derived” from a source, would be a DNA sequence that is identified as a useful fragment, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the disclosure, by the methodology of genetic engineering.

As used herein, a “heterologous” influenza virus gene or viral segment is from an influenza virus source that is different than a majority of the other influenza viral genes or viral segments in a recombinant, e.g., reassortant, influenza virus.

The terms “isolated polypeptide”, “isolated peptide” or “isolated protein” include a polypeptide, peptide or protein encoded by cDNA or recombinant RNA including one of synthetic origin, or some combination thereof.

The term “recombinant protein” or “recombinant polypeptide” as used herein refers to a protein molecule expressed from a recombinant DNA molecule. In contrast, the term “native protein” is used herein to indicate a protein isolated from a naturally occurring (i.e., a nonrecombinant) source. Molecular biological techniques may be used to produce a recombinant form of a protein with identical properties as compared to the native form of the protein.

Methods of alignment of sequences for comparison are well known in the art. Thus, the determination of percent identity between any two sequences can be accomplished using a mathematical algorithm.

Computer implementations of these mathematical algorithms can be utilized for comparison of sequences to determine sequence identity. Alignments using these programs can be performed using the default parameters. Software for performing BLAST analyses is publicly available through the National Center for Biotechnology Information (http://www.ncbi.nlm.nih.gov/). The algorithm may involve first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm may also perform a statistical analysis of the similarity between two sequences. One measure of similarity provided by the BLAST algorithm may be the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

The BLASTN program (for nucleotide sequences) may use as defaults a wordlength (W) of 11, an expectation (E) of 10, a cutoff of 100, M=5, N=−4, and a comparison of both strands. For amino acid sequences, the BLASTP program may use as defaults a wordlength (W) of 3, an expectation (E) of 10, and the BLOSUM62 scoring matrix. See http://www ncbi.n1m.nih.gov. Alignment may also be performed manually by inspection.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

### Influenza Virus Structure and Propagation

Influenza A viruses possess a genome of eight single-stranded negative-sense viral RNAs (vRNAs) that encode at least ten proteins. The influenza virus life cycle begins with binding of the hemagglutinin (HA) to sialic acid-containing receptors on the surface of the host cell, followed by receptor-mediated endocytosis. The low pH in late endosomes triggers a conformational shift in the HA, thereby exposing the N-terminus of the HA2 subunit (the so-called fusion peptide). The fusion peptide initiates the fusion of the viral and endosomal membrane, and the matrix protein (M1) and RNP complexes are released into the cytoplasm. RNPs consist of the nucleoprotein (NP), which encapsidates vRNA, and the viral polymerase complex, which is formed by the PA, PB1, and PB2 proteins. RNPs are transported into the nucleus, where transcription and replication take place. The RNA polymerase complex catalyzes three different reactions: synthesis of an mRNA with a 5′ cap and 3′ polyA structure, of a full-length complementary RNA (cRNA), and of genomic vRNA using the cRNA as a template. Newly synthesized vRNAs, NP, and polymerase proteins are then assembled into RNPs, exported from the nucleus, and transported to the plasma membrane, where budding of progeny virus particles occurs. The neuraminidase (NA) protein plays a crucial role late in infection by removing sialic acid from sialyloligosaccharides, thus releasing newly assembled virions from the cell surface and preventing the self aggregation of virus particles. Although virus assembly involves protein-protein and protein-vRNA interactions, the nature of these interactions is largely unknown.

Although influenza B and C viruses are structurally and functionally similar to influenza A virus, there are some differences. For example, influenza B virus has BM2 and has a viral segment with both NA and NB sequences. Influenza C virus has only seven viral segments.

### Cell Lines That Can Be Used

Any cell, e.g., any avian or mammalian cell, such as a human, e.g., 293T or PER.C6® cells, or canine, e.g., MDCK, bovine, equine, feline, swine, ovine, rodent, for instance mink, e.g., MvLul cells, or hamster, e.g., CHO cells, or non-human primate, e.g., Vero cells, including mutant cells, which supports efficient replication of influenza virus can be employed to isolate and/or propagate influenza viruses. Isolated viruses can be used to prepare a reassortant virus. In embodiments, host cells for vaccine production are continuous mammalian or avian cell lines or cell strains. A complete characterization of the cells to be used, may be conducted so that appropriate tests for purity of the final product can be included. Data that can be used for the characterization of a cell includes (a) information on its origin, derivation, and passage history; (b) information on its growth and morphological characteristics; (c) results of tests of adventitious agents; (d) distinguishing features, such as biochemical, immunological, and cytogenetic patterns which allow the cells to be clearly recognized among other cell lines; and (e) results of tests for tumorigenicity. In embodiments, the passage level, or population doubling, of the host cell used is as low as possible.

In embodiments, the cells are WHO certified, or certifiable, continuous cell lines. The requirements for certifying such cell lines include characterization with respect to at least one of genealogy, growth characteristics, immunological markers, virus susceptibility tumorigenicity and storage conditions, as well as by testing in animals, eggs, and cell culture. Such characterization is used to confirm that the cells are free from detectable adventitious agents. In some countries, karyology may also be required. In addition, tumorigenicity may be tested in cells that are at the same passage level as those used for vaccine production. The virus may be purified by a process that has been shown to give consistent results, before vaccine production (see, e.g., World Health Organization, 1982).

Virus produced by the host cell may be highly purified prior to vaccine or gene therapy formulation. Generally, the purification procedures result in extensive removal of cellular DNA and other cellular components, and adventitious agents. Procedures that extensively degrade or denature DNA may also be used.

### Influenza Vaccines

A vaccine of the disclosure includes an isolated recombinant influenza virus of the disclosure, and optionally one or more other isolated viruses including other isolated influenza viruses, one or more immunogenic proteins or glycoproteins of one or more isolated influenza viruses or one or more other pathogens, e.g., an immunogenic protein from one or more bacteria, non-influenza viruses, yeast or fungi, or isolated nucleic acid encoding one or more viral proteins (e.g., DNA vaccines) including one or more immunogenic proteins of the isolated influenza virus of the disclosure. In embodiments, the influenza viruses of the disclosure may be vaccine vectors for influenza virus or other pathogens.

A complete virion vaccine may be concentrated by ultrafiltration and then purified by zonal centrifugation or by chromatography. Viruses other than the virus of the disclosure, such as those included in a multivalent vaccine, may be inactivated before or after purification using formalin or beta-propiolactone, for instance.

A subunit vaccine comprises purified glycoproteins. Such a vaccine may be prepared as follows: using viral suspensions fragmented by treatment with detergent, the surface antigens are purified, by ultracentrifugation for example. The subunit vaccines thus contain mainly HA protein, and also NA. The detergent used may be cationic detergent for example, such as hexadecyl trimethyl ammonium bromide (Bachmeyer, 1975), an anionic detergent such as ammonium deoxycholate (Laver & Webster, 1976): or a nonionic detergent such as that commercialized under the name TRITON X100. The hemagglutinin may also be isolated after treatment of the virions with a protease such as bromelin, and then purified. The subunit vaccine may be combined with an attenuated virus of the disclosure in a multivalent vaccine.

A split vaccine comprises virions which have been subjected to treatment with agents that dissolve lipids. A split vaccine can be prepared as follows: an aqueous suspension of the purified virus obtained as above, inactivated or not, is treated, under stirring, by lipid solvents such as ethyl ether or chloroform, associated with detergents. The dissolution of the viral envelope lipids results in fragmentation of the viral particles. The aqueous phase is recuperated containing the split vaccine, constituted mainly of hemagglutinin and neuraminidase with their original lipid environment removed, and the core or its degradation products. Then the residual infectious particles are inactivated if this has not already been done. The split vaccine may be combined with an attenuated virus of the disclosure in a multivalent vaccine.

Inactivated Vaccines. Inactivated influenza virus vaccines are provided by inactivating replicated virus using known methods, such as, but not limited to, formalin or β-propiolactone treatment. Inactivated vaccine types that can be used in the disclosure can include whole-virus (WV) vaccines or subvirion (SV) (split) vaccines. The WV vaccine contains intact, inactivated virus, while the SV vaccine contains purified virus disrupted with detergents that solubilize the lipid-containing viral envelope, followed by chemical inactivation of residual virus.

In addition, vaccines that can be used include those containing the isolated HA and NA surface proteins, which are referred to as surface antigen or subunit vaccines.

Live Attenuated Virus Vaccines. Live, attenuated influenza virus vaccines, such as those including a recombinant virus of the disclosure can be used for preventing or treating influenza virus infection. Attenuation may be achieved in a single step by transfer of attenuated genes from an attenuated donor virus to a replicated isolate or reassorted virus according to known methods. Since resistance to influenza A virus is mediated primarily by the development of an immune response to the HA and/or NA glycoproteins, the genes coding for these surface antigens come from the reassorted viruses or clinical isolates. The attenuated genes are derived from an attenuated parent. In this approach, genes that confer attenuation generally do not code for the HA and NA glycoproteins.

Viruses (donor influenza viruses) are available that are capable of reproducibly attenuating influenza viruses, e.g., a cold adapted (ca) donor virus can be used for attenuated vaccine production. Live, attenuated reassortant virus vaccines can be generated by mating the ca donor virus with a virulent replicated virus. Reassortant progeny are then selected at 25° C. (restrictive for replication of virulent virus), in the presence of an appropriate antiserum, which inhibits replication of the viruses bearing the surface antigens of the attenuated ca donor virus. Useful reassortants are: (a) infectious, (b) attenuated for seronegative non-adult mammals and immunologically primed adult mammals, (c) immunogenic and (d) genetically stable. The immunogenicity of the ca reassortants parallels their level of replication. Thus, the acquisition of the six transferable genes of the ca donor virus by new wild-type viruses has reproducibly attenuated these viruses for use in vaccinating susceptible mammals both adults and non-adult.

Other attenuating mutations can be introduced into influenza virus genes by site-directed mutagenesis to rescue infectious viruses bearing these mutant genes. Attenuating mutations can be introduced into non-coding regions of the genome, as well as into coding regions. Such attenuating mutations can also be introduced into genes other than the HA or NA, e.g., the PB2 polymerase gene. Thus, new donor viruses can also be generated bearing attenuating mutations introduced by site-directed mutagenesis, and such new donor viruses can be used in the production of live attenuated reassortants vaccine candidates in a manner analogous to that described above for the ca donor virus. Similarly, other known and suitable attenuated donor strains can be reassorted with influenza virus to obtain attenuated vaccines suitable for use in the vaccination of mammals.

In embodiments, such attenuated viruses maintain the genes from the virus that encode antigenic determinants substantially similar to those of the original clinical isolates. This is because the purpose of the attenuated vaccine is to provide substantially the same antigenicity as the original clinical isolate of the virus, while at the same time lacking pathogenicity to the degree that the vaccine causes minimal chance of inducing a serious disease condition in the vaccinated mammal.

The viruses in a multivalent vaccine can thus be attenuated or inactivated, formulated and administered, according to known methods, as a vaccine to induce an immune response in an animal, e.g., a mammal. Methods are well-known in the art for determining whether such attenuated or inactivated vaccines have maintained similar antigenicity to that of the clinical isolate or high growth strain derived therefrom. Such known methods include the use of antisera or antibodies to eliminate viruses expressing antigenic determinants of the donor virus; chemical selection (e.g., amantadine or rimantidine); HA and NA activity and inhibition; and nucleic acid screening (such as probe hybridization or PCR) to confirm that donor genes encoding the antigenic determinants (e.g., HA or NA genes) are not present in the attenuated viruses.

### Pharmaceutical Compositions

Pharmaceutical compositions of the present disclosure, suitable for inoculation, e.g., nasal, parenteral or oral administration, comprise one or more influenza virus isolates, e.g., one or more attenuated or inactivated influenza viruses, a subunit thereof, isolated protein(s) thereof, and/or isolated nucleic acid encoding one or more proteins thereof, optionally further comprising sterile aqueous or non-aqueous solutions, suspensions, and emulsions. The compositions can further comprise auxiliary agents or excipients, as known in the art. The composition of the disclosure is generally presented in the form of individual doses (unit doses).

Conventional vaccines generally contain about 0.1 to 200 μg, e.g., 30 to 100 μg, of HA from each of the strains entering into their composition. The vaccine forming the main constituent of the vaccine composition of the disclosure may comprise a single influenza virus, or a combination of influenza viruses, for example, at least two or three influenza viruses, including one or more reassortant(s).

Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents.

When a composition of the present disclosure is used for administration to an individual, it can further comprise salts, buffers, adjuvants, or other substances which are desirable for improving the efficacy of the composition. For vaccines, adjuvants, substances which can augment a specific immune response, can be used. Normally, the adjuvant and the composition are mixed prior to presentation to the immune system, or presented separately, but into the same site of the organism being immunized.

Heterogeneity in a vaccine may be provided by mixing replicated influenza viruses for at least two influenza virus strains, such as 2-20 strains or any range or value therein. Vaccines can be provided for variations in a single strain of an influenza virus, using techniques known in the art.

A pharmaceutical composition according to the present disclosure may further or additionally comprise at least one chemotherapeutic compound, for example, for gene therapy, immunosuppressants, anti-inflammatory agents or immune enhancers, and for vaccines, chemotherapeutics including, but not limited to, gamma globulin, amantadine, guanidine, hydroxybenzimidazole, interferon-α, interferon-β, interferon-γ, tumor necrosis factor-alpha, thiosemicarbarzones, methisazone, rifampin, ribavirin, a pyrimidine analog, a purine analog, foscarnet, phosphonoacetic acid, acyclovir, dideoxynucleosides, a protease inhibitor, or ganciclovir.

The composition can also contain variable but small quantities of endotoxin-free formaldehyde, and preservatives, which have been found safe and not contributing to undesirable effects in the organism to which the composition is administered.

### Pharmaceutical Purposes

The administration of the composition (or the antisera that it elicits) may be for either a “prophylactic” or “therapeutic” purpose. When provided prophylactically, the compositions of the disclosure which are vaccines are provided before any symptom or clinical sign of a pathogen infection becomes manifest. The prophylactic administration of the composition serves to prevent or attenuate any subsequent infection. When provided prophylactically, the gene therapy compositions of the disclosure, are provided before any symptom or clinical sign of a disease becomes manifest. The prophylactic administration of the composition serves to prevent or attenuate one or more symptoms or clinical signs associated with the disease.

When provided therapeutically, a viral vaccine is provided upon the detection of a symptom or clinical sign of actual infection. The therapeutic administration of the compound(s) serves to attenuate any actual infection. When provided therapeutically, a gene therapy composition is provided upon the detection of a symptom or clinical sign of the disease. The therapeutic administration of the compound(s) serves to attenuate a symptom or clinical sign of that disease.

Thus, a vaccine composition of the present disclosure may be provided either before the onset of infection (so as to prevent or attenuate an anticipated infection) or after the initiation of an actual infection. Similarly, for gene therapy, the composition may be provided before any symptom or clinical sign of a disorder or disease is manifested or after one or more symptoms are detected.

A composition is said to be “pharmacologically acceptable” if its administration can be tolerated by a recipient mammal. Such an agent is said to be administered in a “therapeutically effective amount” if the amount administered is physiologically significant. A composition of the present disclosure is physiologically significant if its presence results in a detectable change in the physiology of a recipient patient, e.g., enhances at least one primary or secondary humoral or cellular immune response against at least one strain of an infectious influenza virus.

The “protection” provided need not be absolute, i.e., the influenza infection need not be totally prevented or eradicated, if there is a statistically significant improvement compared with a control population or set of mammals. Protection may be limited to mitigating the severity or rapidity of onset of symptoms or clinical signs of the influenza virus infection.

### Pharmaceutical Administration

A composition of the present disclosure may confer resistance to one or more pathogens, e.g., one or more influenza virus strains, by either passive immunization or active immunization. In active immunization, an attenuated live vaccine composition is administered prophylactically to a host (e.g., a mammal), and the host's immune response to the administration protects against infection and/or disease. For passive immunization, the elicited antisera can be recovered and administered to a recipient suspected of having an infection caused by at least one influenza virus strain. A gene therapy composition of the present disclosure may yield prophylactic or therapeutic levels of the desired gene product by active immunization.

In embodiments, the vaccine is provided to a mammalian female (at or prior to pregnancy or parturition), under conditions of time and amount sufficient to cause the production of an immune response which serves to protect both the female and the fetus or newborn (via passive incorporation of the antibodies across the placenta or in the mother's milk).

The present disclosure thus includes methods for preventing or attenuating a disorder or disease, e.g., an infection by at least one strain of pathogen. As used herein, a vaccine is said to prevent or attenuate a disease if its administration results either in the total or partial attenuation (i.e., suppression) of a clinical sign or condition of the disease, or in the total or partial immunity of the individual to the disease. As used herein, a gene therapy composition is said to prevent or attenuate a disease if its administration results either in the total or partial attenuation (i.e., suppression) of a clinical sign or condition of the disease, or in the total or partial immunity of the individual to the disease.

A composition having at least one influenza virus of the present disclosure, including one which is attenuated and one or more other isolated viruses, one or more isolated viral proteins thereof, one or more isolated nucleic acid molecules encoding one or more viral proteins thereof, or a combination thereof, may be administered by any means that achieve the intended purposes.

For example, administration of such a composition may be by various parenteral routes such as subcutaneous, intravenous, intradermal, intramuscular, intraperitoneal, intranasal, oral or transdermal routes. Parenteral administration can be accomplished by bolus injection or by gradual perfusion over time.

A typical regimen for preventing, suppressing, or treating an influenza virus related pathology, comprises administration of an effective amount of a vaccine composition as described herein, administered as a single treatment, or repeated as enhancing or booster dosages, over a period up to and including between one week and about 24 months, or any range or value therein.

According to the present disclosure, an “effective amount” of a composition is one that is sufficient to achieve a desired effect. It is understood that the effective dosage may be dependent upon the species, age, sex, health, and weight of the recipient, kind of concurrent treatment, if any, frequency of treatment, and the nature of the effect wanted. The ranges of effective doses provided below are not intended to limit the disclosure and represent dose ranges.

The dosage of a live, attenuated or killed virus vaccine for an animal such as a mammalian adult organism may be from about 10²-100 μg, e.g., 10³-10¹², plaque forming units (PFU)/kg, or any range or value therein. The dose of inactivated vaccine may range from about 0.1 to 1000, e.g., 30 to 100 μg, of HA protein. However, the dosage should be a safe and effective amount as determined by conventional methods, using existing vaccines as a starting point.

The dosage of immunoreactive HA in each dose of replicated virus vaccine may be standardized to contain a suitable amount, e.g., 30 to 100 μg or any range or value therein, or the amount recommended by government agencies or recognized professional organizations. The quantity of NA can also be standardized, however, this glycoprotein may be labile during purification and storage.

The dosage of immunoreactive HA in each dose of replicated virus vaccine can be standardized to contain a suitable amount, e.g., 1-50 μg or any range or value therein, or the amount recommended by the U.S. Public Health Service (PHS), which is usually 15 μg per component for older children (greater than or equal to 3 years of age), and 7.5 μg per component for children less than 3 years of age. The quantity of NA can also be standardized, however, this glycoprotein can be labile during the processor purification and storage (Kendal et al., 1980; Kerr et al., 1975). Each 0.5-ml dose of vaccine may contains approximately 1-50 billion virus particles, and preferably 10 billion particles.

### Replication-Enhancing Residues

In embodiments, the disclosure provides an isolated recombinant influenza virus having PA, PB1, PB2, NP, NS, and M viral segments from a first influenza vaccine virus isolate, a heterologous, recombinant or chimeric influenza virus NA viral segment, and a heterologous, recombinant or chimeric HA viral segment, wherein one, two or more of the PA, PB1, PB2, NP, NS, and M viral segments have selected amino acid residues at positions 30, 31, 105, 142, 149, 225, 356, 357, 401, and/or 550 in PA; positions 40, 54, 59, 62, e.g., 62, 63, 75, 76, 78, 79, 80, 112, 180, 247, 261, e.g., 161G, 327, 361, e.g., 361R, 507, 577, 621, e.g., 621R, 624, 640, 644, 654, e.g., 654S, 667, 694, 695, 697, 699, 700, 701, 702, 705, 713, and/or 714 in PB1; positions 57, 58, 59, 61, 66, 202, 323, 368, 391, 439, 504, 591, 677, 678, and/or 679, in PB2; positions 74, 112, 116, 224, 293, 371, 377, 417, 422 or 442 in NP; positions 35, 90, 97 and/or 100 in M1; or positions 30, 49, 55, 118, e.g., 118K, 140, 161, and/or 223 in NS1, and optionally an HA with a residue other than glutamic acid, lysine, glutamine, leucine, valine, phenylalanine, lysine or methionine at position 136, 162, 179, 182, 184, 252, 449, or 476, respectively, e.g., a HA segment with one or more of 136D, 162E, 179L, 182V, 184I, 2521, 449E or 476I, or optionally a NA with a residue other than leucine or alanine at residue 55 or 265, respectively, e.g., 55S or 265V.

In embodiments, the isolated virus has one or more of 40L, 62E, 112G, 180W, 247H, 507V, 577R, 624T, 640V, or 644A in PB1; has one or more of 202L, 323L, 439H, or 504V in PB2; has one or more of 74K, 112L, 116L, 417D, or 442A in NP; 35R, 97A and/or 100H in M1; and/or 30P, 55E, 118K and/or 140Q in NS1, or combinations thereof, e.g., has at least one of 202L and/or 323L in PB2, 247H in PB1 or 74K in NP and optionally at least one of 142N in PA1, 55K in NS1 or 97A and/or 100H in M1 or has at least one of 202L and/or 323L in PB2, 247H in PB1 or 74K in NP and at least one of 142N in PA1, 55K in NS1 or 97A and/or 100H in M1. In embodiments, the virus has at least one of 202L and/or 323L in PB2, 247H in PB1 or 74K in NP and optionally at least one of 142N in PA1, 55K in NS1 or 97A and/or 100H in M1. In embodiments, the isolated virus has one or more of 40L, 62E, 180W, 577R, 624T, and/or 640V in PB1; has one or more of 439H or 504V in PB2; may have 116L in NP; may have 35R in M1; and/or 30P and/or 118K in NS1, or combinations thereof.

In embodiments, the isolated virus has 142N, 225C, 356R, or 550L in PA; has one or more of 112G, 247H, 507V, or 644A in PB1; has one or more of 202L, 323L or 504V in PB2; has one or more of 74K, 112L, 116L, 417D, or 442A in NP; 97A and/or 100H in M1; and/or 55E and/or 140Q in NS1, or combinations thereof, e.g., has at least one of 202L and/or 323L in PB2, 247H in PB1 or 74K in NP and optionally at least one of 142N in PA1, 55K in NS1 or 97A and/or 100H in M1 or has at least one of 202L and/or 323L in PB2, 247H in PB1 or 74K in NP and at least one of 142N in PA1, 55K in NS1 or 97A and/or 100H in M1. In embodiments, the virus has at least one of 202L and/or 323L in PB2, 247H in PB1 or 74K in NP and optionally at least one of 142N in PA1, 55K in NS1 or 97A and/or 100H in M1. In embodiments, the virus has at least one of 202L and/or 323L in PB2, 247H in PB1 or 74K in NP and at least one of 142N in PA1, 55K in NS1 or 97A and/or 100H in M1. In embodiments, the isolated virus has 202L and/or 323L in PB2, and optionally has 247H in PB1 and optionally 74K in NP. In embodiments, the isolated virus has 247H in PB1 and optionally 74K in NP. In embodiments, the isolated virus has 401, 40L, 112G, 180W, 247H, 507V, or 644A in PB1 and optionally has 202L and/or 323L in PB2, and optionally has 74K, 112L, 116L, 377N, 417D, or 422L in NP, and optionally has 30P, 118K, 161T or 140Q in NS1, and optionally has 142N, 225C, 356R, 401K, or 550L in PA. In embodiments, the isolated virus has 401, 40L, 112G, 180W, 247H, 507V, or 644A in PB1. In embodiments, the isolated virus has 202L and/or 323L in PB2. In embodiments, the isolated virus has 74K, 112L, I16L, 377N, 417D, or 422L in NP. In embodiments, the isolated virus has 30P, 118K, 161T or 140Q in NS1. In embodiments, the isolated virus has 142N, 225C, 356R, 401K, or 550L in PA. In embodiments, the selected amino acid residues at specified positions in the PA is/are at position(s) 97, 105, 142, 149, 225, 356, 357, 401, 404, and/or 421. In embodiments, the selected amino acid residues at specified positions in the PB1 is/are at position(s) 12, 40, 54, 59, 62, 63, 66, 75, 76, 78, 79, 80, 180, 247, 507, 624, 644, 694, 695, 697, 699, 700, 701, 705, 713, 714, and/or 762. In embodiments, the selected amino acid residues at specified positions in the PB2 is/are at position(s) 57, 58, 59, 61, 66, 202, 243, 323, 504, 677, 678, and/or 679. In embodiments, the selected amino acid residues at specified positions in the NP is/are at position(s) 74, 112, 116, 224, 293, 417, and/or 442. In embodiments, the selected amino acid residues at specified positions in the M1 is/are at position(s) 90, 97, and/or 100. In embodiments, the selected amino acid residues at specified positions in the NS1 is/are at position(s) 49, 30, 55, 161, and/or 223. In embodiments, the selected amino acid residues at specified positions in the PA is/are at position(s) 97, 105, 142, 149, 225, 356, 357, 401, 404, and/or 421; and optionally the selected amino acid residues at specified positions in the PB1 is/are at position(s) 12, 40, 54, 59, 62, 63, 66, 75, 76, 78, 79, 80, 180, 247, 507, 624, 644, 694, 695, 697, 699, 700, 701, 705, 713, 714, and/or 762, in any combination with the selected residues for PA; and optionally the selected amino acid residues at specified positions in the PB2 is/are at position(s) 57, 58, 59, 61, 66, 202, 243, 323, 504, 677, 678, and/or 679 in any combination with the selected residues for PA and/or PB1; and optionally the selected amino acid residues at specified positions in the NP is/are at position(s) 74, 112, 116, 224, 293, 417, and/or 442 any combination with the selected residues for PA, PB1 and/or PB2; and optionally the selected amino acid residues at specified positions in the M1 is/are at position(s) 90, 97, and/or 100 any combination with the selected residues for PA, PB1, PB2, and/or NP; and optionally the selected amino acid residues at specified positions in the NS1 is/are at position(s) 49, 30, 55, 161, and/or 223, or in any combination with the selected residues for PA, PB1, PB2, NP, and/or M1.

For any of the example viruses disclosed above, the PA, PB1, PB2, NP, NS, and M viral segments can comprise sequences for at least one of the following: a PB1 having the amino acid sequence encoded by SEQ ID NO:2 or PB1 with at least 95% amino acid sequence identity to the PB1 encoded by SEQ ID NO:2; a PB2 having the amino acid sequence encoded by SEQ ID NO:3 or PB2 with at least 95% amino acid sequence identity to the PB2 encoded by SEQ ID NO:3; a PA having the amino acid sequence encoded by SEQ ID NO:1 or PA with at least 95% amino acid sequence identity to the PA encoded by SEQ ID NO:1; a NP having the amino acid sequence encoded by SEQ ID NO:4 or NP with at least 95% amino acid sequence identity to the NP encoded by SEQ ID NO:4; a M having the amino acid sequence encoded by SEQ ID NO:5 or M with at least 95% amino acid sequence identity to the M encoded by SEQ ID NO:5; or a NS having the amino acid sequence encoded by SEQ ID NO:6 or NS with at least 95% amino acid sequence identity to the NS encoded by SEQ ID NO:6, or the PA, PB1, PB2, NP, NS, and M viral segments comprise sequences for at least one of the following: a PB1 having the amino acid sequence encoded by SEQ ID NO:10 or PB1 with at least 95% amino acid sequence identity to the PB1 encoded by SEQ ID NO:10; a PB2 having the amino acid sequence encoded by SEQ ID NO:11 or PB2 with at least 95% amino acid sequence identity to the PB2 encoded by SEQ ID NO:11; a PA having the amino acid sequence encoded by SEQ ID NO:12 or PA with at least 95% amino acid sequence identity to the PA encoded by SEQ ID NO:12; a NP having the amino acid sequence encoded by SEQ ID NO:13 or NP with at least 95% amino acid sequence identity to the NP encoded by SEQ ID NO:13; a M having the amino acid sequence encoded by SEQ ID NO:14 or M with at least 95% amino acid sequence identity to the M encoded by SEQ ID NO:14; or a NS having the amino acid sequence encoded by SEQ ID NO: 15 or NS with at least 95% amino acid sequence identity to the NS encoded by SEQ ID NO:15.

For any of the example viruses disclosed above, at least one of the PA, PB1, PB2, NP, NS, and M viral segments can have a C to U promoter mutation.

Any of the isolated viruses disclosed herein may be employed in a vaccine.

In embodiments, the disclosure provides a plurality of influenza virus vectors for preparing a reassortant. In embodiments, the plurality of influenza virus vectors includes a vector for vRNA or cRNA production comprising a promoter operably linked to an influenza virus PA DNA linked to a transcription termination sequence, a vector for vRNA or cRNA production comprising a promoter operably linked to an influenza virus PB1 DNA linked to a transcription termination sequence, a vector for vRNA or cRNA production comprising a promoter operably linked to an influenza virus PB2 DNA linked to a transcription termination sequence, a vector for vRNA or cRNA production comprising a promoter operably linked to an influenza virus HA DNA linked to a transcription termination sequence, a vector for vRNA or cRNA production comprising a promoter operably linked to an influenza virus NP DNA linked to a transcription termination sequence, a vector for vRNA or cRNA production comprising a promoter operably linked to an influenza virus NA DNA linked to a transcription termination sequence, a vector for vRNA or cRNA production comprising a promoter operably linked to an influenza virus M DNA linked to a transcription termination sequence, and a vector for vRNA or cRNA production comprising a promoter operably linked to an influenza virus NS cDNA linked to a transcription termination sequence, wherein the PB1, PB2, PA, NP, NS, and M DNAs in the vectors for vRNA or cRNA production are from one or more influenza vaccine virus isolates, wherein the NA DNA in the vector for vRNA or cRNA production of NA has sequences for a heterologous NA, and wherein the HA DNA in the vector for vRNA or cRNA production of HA has sequences for a heterologous HA, 30, 31, 105, 142, 149, 225, 356, 357, 401, and/or 550 in PA; 40, 54, 59, 62, 63, 75, 76, 78, 79, 80, 112, 180, 247, 327, 507, 577, 624, 640, 644, 667, 694, 695, 697, 699, 700, 701, 702, 705, 713, or 714 and/or 247 in PB1; 57, 58, 59, 61, 66, 202, 323, 368, 391, 439, 504, 591, 677, 678, or 679, 202 and/or 323 in PB2; 74, 112, 116, 224, 293, 371, 377, 417, 422 and/or 442 in NP; 35, 90, 97 and/or 100 in M1; or 30, 49, 55, 118, 140, 161 and/or 223 in NS; and a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, or a vector for mRNA production comprising a promoter operably linked to a DNA segment encoding influenza virus NS2. In embodiments, the PB1, PB2, PA, NP, NS, and M DNAs in the vectors for vRNA or cRNA production have a sequence corresponding to one that encodes a polypeptide having at least 95% amino acid sequence identity to a corresponding polypeptide encoded by SEQ ID NOs:1-6 or 10-15. In embodiments, the promoter for vRNA vectors is a RNA polymerase I promoter, a RNA polymerase II promoter, a RNA polymerase III promoter, a T3 promoter or a T7 promoter. In embodiments, the NA is N9. In embodiments, the HA is H7. In embodiments, the PA, PB1, PB2, NP, NS, and/or M viral segments has/have a promoter C to a mutation.

### Methods

In embodiments, the disclosure provides a method to prepare influenza virus. The method includes contacting a cell with: a vector for vRNA or cRNA production comprising a promoter operably linked to an influenza virus PA DNA linked to a transcription termination sequence, a vector for vRNA or cRNA production comprising a promoter operably linked to an influenza virus PB1 DNA linked to a transcription termination sequence, a vector for vRNA or cRNA production comprising a promoter operably linked to an influenza virus PB2 DNA linked to a transcription termination sequence, a vector for vRNA or cRNA production comprising a promoter operably linked to an influenza virus HA DNA linked to a transcription termination sequence, a vector for vRNA or cRNA production comprising a promoter operably linked to an influenza virus NP DNA linked to a transcription termination sequence, a vector for vRNA or cRNA production comprising a promoter operably linked to an influenza virus NA DNA linked to a transcription termination sequence, a vector for vRNA or cRNA production comprising a promoter operably linked to an influenza virus M DNA linked to a transcription termination sequence, and a vector for vRNA or cRNA production comprising a promoter operably linked to an influenza virus NS DNA linked to a transcription termination sequence, wherein the PB1, PB2, PA, NP, NS, and M DNAs in the vectors for vRNA or cRNA production are from one or more influenza vaccine virus isolates, wherein the NA DNA in the vector for vRNA or cRNA production of NA has sequences for a heterologous NA, and wherein the HA DNA in the vector for vRNA or cRNA production of HA has sequences for a heterologous HA, 30, 31, 105, 142, 149, 225, 356, 357, 401, and/or 550 in PA; 40, 54, 59, 62, 63, 75, 76, 78, 79, 80, 112, 180, 247, 327, 507, 577, 624, 640,644, 667, 694, 695, 697, 699, 700, 701, 702, 705, 713, and/or 714 and/or 247 in PB1; 57, 58, 59, 61, 66, 202, 323, 368, 391, 504, 591, 677, 678, and/or 679, 202 and/or 323 in PB2; 74, 112, 116, 224, 293, 371, 377, 417, 422 and/or 442 in NP; 35, 90, 97 and/or 100 in M1; or 30, 49, 55, 118, 140, 161 or 223 in NS; and a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, or a vector for mRNA production comprising a promoter operably linked to a DNA segment encoding influenza virus NS2; in an amount effective to yield infectious influenza virus. In embodiments, the cell is an avian cell or a mammalian cell, e.g., a Vero cell, a human cell or a MDCK cell. In embodiments, the PB1, PB2, PA, NP, NS, and M DNAs in the vectors for vRNA or cRNA productions have a sequence that corresponds to one that encodes a polypeptide having at least 95% amino acid sequence identity to a corresponding polypeptide encoded by SEQ ID NOs:1-6 or 10-15. In embodiments, the method includes isolating the virus. In embodiments, at least one of PA, PB1, or PB2 viral segments has a C to U promoter mutation.

Further provided is a vector for vRNA or mRNA expression of influenza virus PA having at least 95% amino acid sequence identity to a polypeptide encoded by SEQ ID NO:1 and having a threonine at position 30, a lysine at position 31, cysteine at position 105 or a lysine at position 401, or a combination thereof; a vector for vRNA or mRNA expression of influenza virus PB1 having at least 95% amino acid sequence identity to a polypeptide encoded by SEQ ID NO:2 and having a leucine at position 40, an alanine or isoleucine at position 54, a glutamic acid at position 62, glycine at position 112, histidine at position 247, valine at position 507, arginine at position 577, valine at position 640, isoleucine at position 624, alanine at position 644, or cysteine at position 713, or any combination thereof; a vector for vRNA or mRNA expression of PB2 having at least 95% amino acid sequence identity to a polypeptide encoded by SEQ ID NO:3 and a leucine at position 202 and/or 323 or a valine at position 504, or a combination thereof, a vector for vRNA or mRNA expression of influenza virus NP having at least 95% amino acid sequence identity to a polypeptide encoded by SEQ ID NO:4 and having a lysine at position 74, leucine at position 116, isoleucine at position 224, lysine at position 293, asparagine at position 377, or aspartic acid at position 417, or any combination thereof; a vector for vRNA or mRNA expression of influenza virus NS1 having at least 95% amino acid sequence identity to a NS1 polypeptide encoded by SEQ ID NO:6 and having a proline at position 30, alanine at position 49, lysine at position 118, glutamine at position 140, threonine at position 161, or glutamic acid at position 223, or any combination thereof; and a vector for vRNA or mRNA expression of influenza virus M1 having at least 95% amino acid sequence identity to a M1 polypeptide encoded by SEQ ID NO:5 and having an arginine at position 35, or any combination thereof.

### Example Methods

### Cells and Viruses

293T human embryonic kidney cells are maintained in Dulbecco's modified Eagle's minimal essential medium (DMEM) with 10% fetal calf serum and antibiotics. Madin-Darby canine kidney (MDCK) cells are grown in MEM with 5% newborn calf serum and antibiotics. African green monkey Vero WCB cells, which had been established after biosafety tests for use in human vaccine production (Sugawara et al., 2002), are maintained in serum-free VP-SFM medium (GIBCO-BRL) with antibiotics. Cells are maintained at 37° C. in 5% CO₂. A WHO-recommended vaccine seed virus is NIBRG-14.

### Construction of Plasmids and Reverse Genetics

To generate reassortants of influenza A viruses, a plasmid-based reverse genetics (Neumann et al., 1999) is used. The full-length cDNAs were cloned into a plasmid under control of the human polymerase I promoter and the mouse RNA polymerase I terminator (PolI plasmids).

A previously produced series of PolI constructs, derived from A/WSN/33 (H5N1; WSN) or PR8 strains is used, for reverse genetics (Horimoto et al., 2006; Neumann et al., 1999). The World Health Organization (WHO) recommends A/Puerto Rico/8/34 (H1N1; PR8) as a donor virus, because of its safety in humans (Wood & Robertson, 2004; Webby & Webster, 2003).

Plasmids expressing WSN or PR8 NP, PA, PB1, or PB2 under control of the chicken actin, e.g., beta-actin, promoter are used for all reverse genetics experiments (Horimoto et al., 2006; Neumann et al., 1999). Briefly, PolI plasmids and protein expression plasmids are mixed with a transfection reagent, Trans-IT 293T (Panvera), incubated at room temperature for 15 minutes, and then added to 293T cells. Transfected cells are incubated in Opti-MEM I (GIBCO-BRL) for 48 hours. For reverse genetics in Vero WCB cells, an electroporator (Amaxa) is used to transfect the plasmid mixtures according to the manufacturer's instructions. Sixteen hours after transfection, freshly prepared Vero WCB cells were added onto the transfected cells and TPCK-trypsin (1 μg/mL) is added to the culture 6 hours later. Transfected cells are incubated in serum-free VP-SFM for a total of 4 days. Supernatants containing infectious viruses are harvested and may be biologically cloned by limiting dilution.

A recombinant virus having the HA and NA genes from A/Hong Kong/213/2003 (H5N1) and the remainder of the type A influenza virus genes from PR8(UW) was prepared. The titer of the recombinant virus was 10^{10.67} EID₅₀/mL, and the HA titer was 1:1600.

TABLE A
HA titers of influenza A virus with PR8 genes
with WSN-HA NA genes or HK-HAavir NA genes.

Virus possessing
PR8 genes together

| with the following | HA titer (HAU/mL) in each dilition | | | | | | |
|---|---|---|---|---|---|---|---|
| HA and NA genes | 10-2 | 10-3 | 10-4 | 10-5 | 10-6 | 10-7 | 10-8 |
| WSN-HA NA | 160 | 40 | 40 | 320 | 40 | 640 | <1 |
| HK-HAavir NA | 400 | 800 | 400 | 400 | 400 | 800 | <1 |

The nucleic acid sequences of PR8 (UW) genes are as follows:

PA
(SEQ ID NO: 1)
AGCGAAAGCA GGTACTGATC CAAAATGGAA GATTTTGTGC
GACAATGCTT CAATCCGATG ATTGTCGAGC TTGCGGAAAA
AACAATGAAA GAGTATGGGG AGGACCTGAA AATCGAAACA
AACAAATTTG CAGCAATATG CACTCACTTG GAAGTATGCT
TCATGTATTC AGATTTTCAC TTCATCAATG AGCAAGGCGA
GTCAATAATC GTAGAACTTG GTGATCCAAA TGCACTTTTG
AAGCACAGAT TTGAAATAAT CGAGGGAAGA GATCGCACAA
TGGCCTGGAC AGTAGTAAAC AGTATTTGCA ACACTACAGG
GGCTGAGAAA CCAAAGTTTC TACCAGATTT GTATGATTAC
AAGGAGAATA GATTCATCGA AATTGGAGTA ACAAGGAGAG
AAGTTCACAT ATACTATCTG GAAAAGGCCA ATAAAATTAA
ATCTGAGAAA ACACACATCC ACATTTTCTC GTTCACTGGG
GAAGAAATGG CCACAAAGGC AGACTACACT CTCGATGAAG
AAAGCAGGGC TAGGATCAAA ACCAGACTAT TCACCATAAG
ACAAGAAATG GCCAGCAGAG GCCTCTGGGA TTCCTTTCGT
CAGTCCGAGA GAGGAGAAGA GACAATTGAA GAAAGGTTTG
AAATCACAGG AACAATGCGC AAGCTTGCCG ACCAAAGTCT
CCCGCCGAAC TTCTCCAGCC TTGAAAATTT TAGAGCCTAT
GTGGATGGAT TCGAACCGAA CGGCTACATT GAGGGCAAGC
TGTCTCAAAT GTCCAAAGAA GTAAATGCTA GAATTGAACC
TTTTTTGAAA ACAACACCAC GACCACTTAG ACTTCCGAAT
GGGCCTCCCT GTTCTCAGCG GTCCAAATTC CTGCTGATGG
ATGCCTTAAA ATTAAGCATT GAGGACCCAA GTCATGAAGG
AGAGGGAATA CCGCTATATG ATGCAATCAA ATGCATGAGA
ACATTCTTTG GATGGAAGGA ACCCAATGTT GTTAAACCAC
ACGAAAAGGG AATAAATCCA AATTATCTTC TGTCATGGAA
GCAAGTACTG GCAGAACTGC AGGACATTGA GAATGAGGAG
AAAATTCCAA AGACTAAAAA TATGAAGAAA ACAAGTCAGC
TAAAGTGGGC ACTTGGTGAG AACATGGCAC CAGAAAAGGT
AGACTTTGAC GACTGTAAAG ATGTAGGTGA TTTGAAGCAA
TATGATAGTG ATGAACCAGA ATTGAGGTCG CTTGCAAGTT
GGATTCAGAA TGAGTTTAAC AAGGCATGCG AACTGACAGA
TTCAAGCTGG ATAGAGCTCG ATGAGATTGG AGAAGATGTG
GCTCCAATTG AACACATTGC AAGCATGAGA AGGAATTATT
TCACATCAGA GGTGTCTCAC TGCAGAGCCA CAGAATACAT
AATGAAGGGA GTGTACATCA ATACTGCCTT GCTTAATGCA
TCTTGTGCAG CAATGGATGA TTTCCAATTA ATTCCAATGA
TAAGCAAGTG TAGAACTAAG GAGGGAAGGC GAAAGACCAA
CTTGTATGGT TTCATCATAA AAGGAAGATC CCACTTAAGG
AATGACACCG ACGTGGTAAA CTTTGTGAGC ATGGAGTTTT
CTCTCACTGA CCCAAGACTT GAACCACATA AATGGGAGAA
GTACTGTGTT CTTGAGATAG GAGATATGCT TATAAGAAGT
GCCATAGGCC AGGTTTCAAG GCCCATGTTC TTGTATGTGA
GAACAAATGG AACCTCAAAA ATTAAAATGA AATGGGGAAT
GGAGATGAGG CGTTGCCTCC TCCAGTCACT TCAACAAATT
GAGAGTATGA TTGAAGCTGA GTCCTCTGTC AAAGAGAAAG
ACATGACCAA AGAGTTCTTT GAGAACAAAT CAGAAACATG
GCCCATTGGA GAGTCCCCCA AAGGAGTGGA GGAAAGTTCC
ATTGGGAAGG TCTGCAGGAC TTTATTAGCA AAGTCGGTAT
TCAACAGCTT GTATGCATCT CCACAACTAG AAGGATTTTC
AGCTGAATCA AGAAAACTGC TTCTTATCGT TCAGGCTCTT
AGGGACAACC TGGAACCTGG GACCTTTGAT CTTGGGGGGC
TATATGAAGC AATTGAGGAG TGCCTGATTA ATGATCCCTG
GGTTTTGCTT AATGCTTCTT GGTTCAACTC CTTCCTTACA
CATGCATTGA GTTAGTTGTG GCAGTGCTAC TATTTGCTAT
CCATACTGTC CAAAAAAGTA CCTTGTTTCT ACT
PB1
(SEO ID NO: 2)
AGCGAAAGCA GGCAAACCAT TTGAATGGAT GTCAATCCGA
CCTTACTTTT CTTAAAAGTG CCAGCACAAA ATGCTATAAG
CACAACTTTC CCTTATACTG GAGACCCTCC TTACAGCCAT
GGGACAGGAA CAGGATACAC CATGGATACT GTCAACAGGA
CACATCAGTA CTCAGAAAAG GGAAGATGGA CAACAAACAC
CGAAACTGGA GCACCGCAAC TCAACCCGAT TGATGGGCCA
CTGCCAGAAG ACAATGAACC AAGTGGTTAT GCCCAAACAG
ATTGTGTATT GGAGGCGATG GCTTTCCTTG AGGAATCCCA
TCCTGGTATT TTTGAAAACT CGTGTATTGA AACGATGGAG
GTTGTTCAGC AAACACGAGT AGACAAGCTG ACACAAGGCC
GACAGACCTA TGACTGGACT CTAAATAGAA ACCAACCTGC
TGCAACAGCA TTGGCCAACA CAATAGAAGT GTTCAGATCA
AATGGCCTCA CGGCCAATGA GTCTGGAAGG CTCATAGACT
TCCTTAAGGA TGTAATGGAG TCAATGAACA AAGAAGAAAT
GGGGATCACA ACTCATTTTC AGAGAAAGAG ACGGGTGAGA
GACAATATGA CTAAGAAAAT GATAACACAG AGAACAATGG
GTAAAAAGAA GCAGAGATTG AACAAAAGGA GTTATCTAAT
TAGAGCATTG ACCCTGAACA CAATGACCAA AGATGCTGAG
AGAGGGAAGC TAAAACGGAG AGCAATTGCA ACCCCAGGGA
TGCAAATAAG GGGGTTTGTA TACTTTGTTG AGACACTGGC
AAGGAGTATA TGTGAGAAAC TTGAACAATC AGGGTTGCCA
GTTGGAGGCA ATGAGAAGAA AGCAAAGTTG GCAAATGTTG
TAAGGAAGAT GATGACCAAT TCTCAGGACA CCGAACTTTC
TTTCACCATC ACTGGAGATA ACACCAAATG GAACGAAAAT
CAGAATCCTC GGATGTTTTT GGCCATGATC ACATATATGA
CCAGAAATCA GCCCGAATGG TTCAGAAATG TTCTAAGTAT
TGCTCCAATA ATGTTCTCAA ACAAAATGGC GAGACTGGGA
AAAGGGTATA TGTTTGAGAG CAAGAGTATG AAACTTAGAA
CTCAAATACC TGCAGAAATG CTAGCAAGCA TCGATTTGAA
ATATTTCAAT GATTCAACAA GAAAGAAGAT TGAAAAAATC
CGACCGCTCT TAATAGAGGG GACTGCATCA TTGAGCCCTG
GAATGATGAT GGGCATGTTC AATATGTTAA GCACTGTATT
AGGCGTCTCC ATCCTGAATC TTGGACAAAA GAGATACACC
AAGACTACTT ACTGGTGGGA TGGTCTTCAA TCCTCTGACG
ATTTTGCTCT GATTGTGAAT GCACCCAATC ATGAAGGGAT
TCAAGCCGGA GTCGACAGGT TTTATCGAAC CTGTAAGCTA
CTTGGAATCA ATATGAGCAA GAAAAAGTCT TACATAAACA
GAACAGGTAC ATTTGAATTC ACAAGTTTTT TCTATCGTTA
TGGGTTTGTT GCCAATTTCA GCATGGAGCT TCCCAGTTTT
GGGGTGTCTG GGATCAACGA GTCAGCGGAC ATGAGTATTG
GAGTTACTGT CATCAAAAAC AATATGATAA ACAATGATCT
TGGTCCAGCA ACAGCTCAAA TGGCCCTTCA GTTGTTCATC
AAAGATTACA GGTACACGTA CCGATGCCAT ATAGGTGACA
CACAAATACA AACCCGAAGA TCATTTGAAA TAAAGAAACT
GTGGGAGCAA ACCCGTTCCA AAGCTGGACT GCTGGTCTCC
GACGGAGGCC CAAATTTATA CAACATTAGA AATCTCCACA
TTCCTGAAGT CTGCCTAAAA TGGGAATTGA TGGATGAGGA
TTACCAGGGG CGTTTATGCA ACCCACTGAA CCCATTTGTC
AGCCATAAAG AAATTGAATC AATGAACAAT GCAGTGATGA
TGCCAGCACA TGGTCCAGCC AAAAACATGG AGTATGATGC
TGTTGCAACA ACACACTCCT GGATCCCCAA AAGAAATCGA
TCCATCTTGA TACAAGTCA AAGAGGAGTA CTTGAGGATG
AACAAATGTA CCAAAGGTGC TGCAATTTAT TTGAAAAATT
CTTCCCCAGC AGTTCATACA GAAGACCAGT CGGGATATCC
AGTATGGTGG AGGCTATGGT TTCCAGAGCC CGAATTGATG
CACGGATTGA TTTCGAATCT GGAAGGATAA AGAAAGAAGA
GTTCACTGAG ATCATGAAGA TCTGTTCCAC CATTGAAGAG
CTCAGACGGC AAAAATAGTG AATTTAGCTT GTCCTTCATG
AAAAAATGCC TTGTTTCTAC T
PB2
(SEQ ID NO: 3)
AGCGAAAGCA GGTCAATTAT ATTCAATATG GAAAGAATAA
AAGAACTACG AAATCTAATG TCGCAGTCTC GCACCCGCGA
GATACTCACA AAAACCACCG TGGACCATAT GGCCATAATC
AAGAAGTACA CATCAGGAAG ACAGGAGAAG AACCCAGCAC
TTAGGATGAA ATGGATGATG GCAATGAAAT ATCCAATTAC
AGCAGACAAG AGGATAACGG AAATGATTCC TGAGAGAAAT
GAGCAAGGAC AAACTTTATG GAGTAAAATG AATGATGCCG
GATCAGACCG AGTGATGGTA TCACCTCTGG CTGTGACATG
GTGGAATAGG AATGGACCAA TAACAAATAC AGTTCATTAT
CCAAAAATCT ACAAAACTTA TTTTGAAAGA GTCGAAAGGC
TAAAGCATGG AACCTTTGGC CCTGTCCATT TTAGAAACCA
AGTCAAAATA CGTCGGAGAG TTGACATAAA TCCTGGTCAT
GCAGATCTCA GTGCCAAGGA GGCACAGGAT GTAATCATGG
AAGTTGTTTT CCCTAACGAA GTGGGAGCCA GGATACTAAC
ATCGGAATCG CAACTAACGA TAACCAAAGA GAAGAAAGAA
GAACTCCAGG ATTGCAAAAT TTCTCCTTTG ATGGTTGCAT
ACATGTTGGA GAGAGAACTG GTCCGCAAAA CGAGATTCCT
CCCAGTGGCT GGTGGAACAA GCAGTGTGTA CATTGAAGTG
TTGCATTTGA CTCAAGGAAC ATGCTGGGAA CAGATGTATA
CTCCAGGAGG GGAAGTGAGG AATGATGATG TTGATCAAAG
CTTGATTATT GCTGCTAGGA ACATAGTGAG AAGAGCTGCA
GTATCAGCAG ATCCACTAGC ATCTTTATTG GAGATGTGCC
ACAGCACACA GATTGGTGGA ATTAGGATGG TAGACATCCT
TAGGCAGAAC CCAACAGAAG AGCAAGCCGT GGATATATGC
AAGGCTGCAA TGGGACTGAG AATTAGCTCA TCCTTCAGTT
TTGGTGGATT CACATTTAAG AGAACAAGCG GATCATCAGT
CAAGAGAGAG GAAGAGGTGC TTACGGGCAA TCTTCAAACA
TTGAAGATAA GAGTGCATGA GGGATATGAA GAGTTCACAA
TGGTTGGGAG AAGAGCAACA GCCATACTCA GAAAAGCAAC
CAGGAGATTG ATTCAGCTGA TAGTGAGTGG GAGAGACGAA
CAGTCGATTG CCGAAGCAAT AATTGTGGCC ATGGTATTTT
CACAAGAGGA TTGTATGATA AAAGCAGTCA GAGGTGATCT
GAATTTCGTC AATAGGGCGA ATCAACGATT GAATCCTATG
CATCAACTTT TAAGACATTT TCAGAAGGAT GCGAAAGTGC
TTTTTCAAAA TTGGGGAGTT GAACCTATCG ACAATGTGAT
GGGAATGATT GGGATATTGC CCGACATGAC TCCAAGCATC
GAGATGTCAA TGAGAGGAGT GAGAATCAGC AAAATGGGTG
TAGATGAGTA CTCCAGCACG GAGAGGGTAG TGGTGAGCAT
TGACCGTTTT TTGAGAATCC GGGACCAACG AGGAAATGTA
CTACTGTCTC CCGAGGAGGT CAGTGAAACA CAGGGAACAG
AGAAACTGAC AATAACTTAC TCATCGTCAA TGATGTGGGA
GATTAATGGT CCTGAATCAG TGTTGGTCAA TACCTATCAA
TGGATCATCA GAAACTGGGA AACTGTTAAA ATTCAGTGGT
CCCAGAACCC TACAATGCTA TACAATAAAA TGGAATTTGA
ACCATTTCAG TCTTTAGTAC CTAAGGCCAT TAGAGGCCAA
TACAGTGGGT TTGTAAGAAC TCTGTTCCAA CAAATGAGGG
ATGTGCTTGG GACATTTGAT ACCGCACAGA TAATAAAACT
TCTTCCCTTC GCAGCCGCTC CACCAAAGCA AAGTAGAATG
CAGTTCTCCT CATTTACTGT GAATGTGAGG GGATCAGGAA
TGAGAATACT TGTAAGGGGC AATTCTCCTG TATTCAACTA
TAACAAGGCC ACGAAGAGAC TCACAGTTCT CGGAAAGGAT
GCTGGCACTT TAACTGAAGA CCCAGATGAA GGCACAGCTG
GAGTGGAGTC CGCTGTTCTG AGGGGATTCC TCATTCTGGG
CAAAGAAGAC AAGAGATATG GGCCAGCACT AAGCATCAAT
GAACTGAGCA ACCTTGCGAA AGGAGAGAAG GCTAATGTGC
TAATTGGGCA AGGAGACGTG GTGTTGGTAA TGAAACGGAA
ACGGGACTCT AGCATACTTA CTGACAGCCA GACAGCGACC
AAAAGAATTC GGATGGCCAT CAATTAGTGT CGAATAGTTT
AAAAACGACC TTGTTTCTAC T
NP
(SEQ ID NO: 4)
AGCAAAAGCA GGGTAGATAA TCACTCACTG AGTGACATCA
AAATCATGGC GTCTCAAGGC ACCAAACGAT CTTACGAACA
GATGGAGACT GATGGAGAAC GCCAGAATGC CACTGAAATC
AGAGCATCCG TCGGAAAAAT GATTGGTGGA ATTGGACGAT
TCTACATCCA AATGTGCACC GAACTCAAAC TCAGTGATTA
TGAGGGACGG TTGATCCAAA ACAGCTTAAC AATAGAGAGA
ATGGTGCTCT CTGCTTTTGA CGAAAGGAGA AATAAATACC
TTGAAGAACA TCCCAGTGCG GGGAAAGATC CTAAGAAAAC
TGGAGGACCT ATATACAGGA GAGTAAACGG AAAGTGGATG
AGAGAACTCA TCCTTTATGA CAAAGAAGAA ATAAGGCGAA
TCTGGCGCCA AGCTAATAAT GGTGACGATG CAACGGCTGG
TCTGACTCAC ATGATGATCT GGCATTCCAA TTTGAATGAT
GCAACTTATC AGAGGACAAG AGCTCTTGTT CGCACCGGAA
TGGATCCCAG GATGTGCTCT CTGATGCAAG GTTCAACTCT
CCCTAGGAGG TCTGGAGCCG CAGGTGCTGC AGTCAAAGGA
GTTGGAACAA TGGTGATGGA ATTGGTCAGA ATGATCAAAC
GTGGGATCAA TGATCGGAAC TTCTGGAGGG GTGAGAATGG
ACGAAAAACA AGAATTGCTT ATGAAAGAAT GTGCAACATT
CTCAAAGGGA AATTTCAAAC TGCTGCACAA AAAGCAATGA
TGGATCAAGT GAGAGAGAGC CGGAACCCAG GGAATGCTGA
GTTCGAAGAT CTCACTTTTC TAGCACGGTC TGCACTCATA
TTGAGAGGGT CGGTTGCTCA CAAGTCCTGC CTGCCTGCCT
GTGTGTATGG ACCTGCCGTA GCCAGTGGGT ACGACTTTGA
AAGGGAGGGA TACTCTCTAG TCGGAATAGA CCCTTTCAGA
CTGCTTCAAA ACAGCCAAGT GTACAGCCTA ATCAGACCAA
ATGAGAATCC AGCACACAAG AGTCAACTGG TGTGGATGGC
ATGCCATTCT GCCGCATTTG AAGATCTAAG AGTATTAAGC
TTCATCAAAG GGACGAAGGT GCTCCCAAGA GGGAAGCTTT
CCACTAGAGG AGTTCAAATT GCTTCCAATG AAAATATGGA
GACTATGGAA TCAAGTACAC TTGAACTGAG AAGCAGGTAC
TGGGCCATAA GGACCAGAAG TGGAGGAAAC ACCAATCAAC
AGAGGGCATC TGCGGGCCAA ATCAGCATAC AACCTACGTT
CTCAGTACAG AGAAATCTCC CTTTTGACAG AACAACCATT
ATGGCAGCAT TCAATGGGAA TACAGAGGGG AGAACATCTG
ACATGAGGAC CGAAATCATA AGGATGATGG AAAGTGCAAG
ACCAGAAGAT GTGTCTTTCC AGGGGGGGGG AGTCTTCGAG
CTCTCGGACG AAAAGGCAGC GAGCCCGATC GTGCCTTCCT
TTGACATGAG TAATGAAGGA TCTTATTTCT TCGGAGACAA
TGCAGAGGAG TACGACAATT AAAGAAAAAT ACCCTTGTTT
CTACT
M
(SEQ ID NO: 5)
AGCAAAAGCA GGTAGATATT GAAAGATGAG TCTTCTAACC
GAGGTCGAAA CGTACGTACT CTCTATCATC CCGTCAGGCC
CCCTCAAAGC CGAGATCGCA CAGAGACTTG AAGATGTCTT
TGCAGGGAAG AACACCGATC TTGAGGTTCT CATGGAATGG
CTAAAGACAA GACCAATCCT GTCACCTCTG ACTAAGGGGA
TTTTAGGATT TGTGTTCACG CTCACCGTGC CCAGTGAGCG
AGGACTGCAG CGTAGACGCT TTGTCCAAAA TGCCCTTAAT
GGGAACGGGG ATCCAAATAA CATGGACAAA GCAGTTAAAC
TGTATAGGAA GCTCAAGAGG GAGATAACAT TCCATGGGGC
CAAAGAAATC TCACTCAGTT ATTCTGCTGG TGCACTTGCC
AGTTGTATGG GCCTCATATA CAACAGGATG GGGGCTGTGA
CCACTGAAGT GGCATTTGGC CTGGTATGTG CAACCTGTGA
ACAGATTGCT GACTCCCAGC ATCGGTCTCA TAGGCAAATG
GTGACAACAA CCAATCCACT AATCAGACAT GAGAACAGAA
TGGTTTTAGC CAGCACTACA GCTAAGGCTA TGGAGCAAAT
GGCTGGATCG AGTGAGCAAG CAGCAGAGGC CATGGAGGTT
GCTAGTCAGG CTAGACAAAT GGTGCAAGCG ATGAGAACCA
TTGGGACTCA TCCTAGCTCC AGTGCTGGTC TGAAAAATGA
TCTTCTTGAA AATTTGCAGG CCTATCAGAA ACGAATGGGG
GTGCAGATGC AACGGTTCAA GTGATCCTCT CACTATTGCC
GCAAATATCA TTGGGATCTT GCACTTGACA TTGTGGATTC
TTGATCGTCT TTTTTTCAAA TGCATTTACC GTCGCTTTAA
ATACGGACTG AAAGGAGGGC CTTCTACGGA AGGAGTGCCA
AAGTCTATGA GGGAAGAATA TCGAAAGGAA CAGCAGAGTG
CTGTGGATGC TGACGATGGT CATTTTGTCA GCATAGAGCT
GGAGTAAAAA ACTACCTTGT TTCTACT
NS
(SEQ ID NO: 6)
AGCAAAAGCA GGGTGACAAA AACATAATGG ATCCAAACAC
TGTGTCAAGC TTTCAGGTAG ATTGCTTTCT TTGGCATGTC
CGCAAACGAG TTGCAGACCA AGAACTAGGC GATGCCCCAT
TCCTTGATCG GCTTCGCCGA GATCAGAAAT CCCTAAGAGG
AAGGGGCAGT ACTCTCGGTC TGGACATCAA GACAGCCACA
CGTGCTGGAA AGCAGATAGT GGAGCGGATT CTGAAAGAAG
AATCCGATGA GGCACTTAAA ATGACCATGG CCTCTGTACC
TGCGTCGCGT TACCTAACTG ACATGACTCT TGAGGAAATG
TCAAGGGACT GGTCCATGCT CATACCCAAG CAGAAAGTGG
CAGGCCCTCT TTGTATCAGA ATGGACCAGG CGATCATGGA
TAAGAACATC ATACTGAAAG CGAACTTCAG TGTGATTTTT
GACCGGCTGG AGACTCTAAT ATTGCTAAGG GCTTTCACCG
AAGAGGGAGC AATTGTTGGC GAAATTTCAC CATTGCCTTC
TCTTCCAGGA CATACTGCTG AGGATGTCAA AAATGCAGTT
GGAGTCCTCA TCGGAGGACT TGAATGGAAT GATAACACAG
TTCGAGTCTC TGAAACTCTA CAGAGATTCG CTTGGAGAAG
CAGTAATGAG AATGGGAGAC CTCCACTCAC TCCAAAACAG
AAACGAGAAA TGGGGGGAAC AATTAGGTCA GAAGTTTGAA
GAAATAAGAT GGTTGATTGA AGAAGTGAGA CACAAACTGA
AGATAACAGA GAATAGTTTT GAGCAAATAA CATTTATGCA
AGCCTTACAT CTATTGCTTG AAGTGGAGCA AGAGATAAGA
ACTTTCTCGT TTCAGCTTAT TTAGTACTAA AAAACACCCT
TGTTTCTACT
HA
(SEQ ID NO: 7)
AGCAAAAGCAGGGGAAAATAAAAACAACCAAAATGAAGGCAAAC
CTACTGGTCCTGTTATGTGCACTTGCAGCTGCAGATGCAGACAC
AATATGTATAGGCTACCATGCGAACAATTCAACCGACACTGTTG
ACACAGTACTCGAGAAGAATGTGACAGTGACACACTCTGTTAAC
CTGCTCGAAGACAGCCACAACGGAAAACTATGTAGATTAAAAGG
AATAGCCCCACTACAATTGGGGAAATGTAACATCGCCGGATGGC
TCTTGGGAAACCCAGAATGCGACCCACTGCTTCCAGTGAGATCA
TGGTCCTACATTGTAGAAACACCAAACTCTGAGAATGGAATATG
TTATCCAGGAGATTTCATCGACTATGAGGAGCTGAGGGAGCAAT
TGAGCTCAGTGTCATCATTCGAAAGATTCGAAATATTTCCCAAA
GAAAGCTCATGGCCCAACCACAACACAAACGGAGTAACGGCAGC
ATGCTCCCATGAGGGGAAAAGCAGTTTTTACAGAAATTTGCTAT
GGCTGACGGAGAAGGAGGGCTCATACCCAAAGCTGAAAAATTCT
TATGTGAACAAAAAAGGGAAAGAAGTCCTTGTACTGTGGGGTAT
TCATCACCCGCCTAACAGTAAGGAACAACAGAATCTCTATCAGA
ATGAAAATGCTTATGTCTCTGTAGTGACTTCAAATTATAACAGG
AGATTTACCCCGGAAATAGCAGAAAGACCCAAAGTAAGAGATCA
AGCTGGGAGGATGAACTATTACTGGACCTTGCTAAAACCCGGAG
ACACAATAATATTTGAGGCAAATGGAAATCTAATAGCACCAATG
TATGCTTTCGCACTGAGTAGAGGCTTTGGGTCCGGCATCATCAC
CTCAAACGCATCAATGCATGAGTGTAACACGAAGTGTCAAACAC
CCCTGGGAGCTATAAACAGCAGTCTCCCTTACCAGAATATACAC
CCAGTCACAATAGGAGAGTGCCCAAAATACGTCAGGAGTGCCAA
ATTGAGGATGGTTACAGGACTAAGGAACATTCCGTCCATTCAAT
CCAGAGGTCTATTTGGAGCCATTGCCGGTTTTATTGAAGGGGGA
TGGACTGGAATGATAGATGGATGGTATGGTTATCATCATCAGAA
TGAACAGGGATCAGGCTATGCAGCGGATCAAAAAAGCACACAAA
ATGCCATTAACGGGATTACAAACAAGGTGAACACTGTTATCGAG
AAAATGAACATTCAATTCACAGCTGTGGGTAAAGAATTCAACAA
ATTAGAAAAAAGGATGGAAAATTTAAATAAAAAAGTTGATGATG
GATTTCTGGACATTTGGACATATAATGCAGAATTGTTAGTTCTA
CTGGAAAATGAAAGGACTCTGGATTTCCATGACTCAAATGTGAA
GAATCTGTATGAGAAAGTAAAAAGCCAATTAAAGAATAATGCCA
AAGAAATCGGAAATGGATGTTTTGAGTTCTACCACAAGTGTGAC
AATGAATGCATGGAAAGTGTAAGAAATGGGACTTATGATTATCC
CAAATATTCAGAAGAGTCAAAGTTGAACAGGGAAAAGGTAGATG
GAGTGAAATTGGAATCAATGGGGATCTATCAGATTCTGGCGATC
TACTCAACTGTCGCCAGTTCACTGGTGCTTTTGGTCTCCCTGGG
GGCAATCAGTTTCTGGATGTGTTCTAATGGATCTTTGCAGTGCA
GAATATGCATCTGAGATTAGAATTTCAGAGATATGAGGAAAAAC
ACCCTTGTTTCTACT
NA
(SEQ ID NO: 8)
AGCAAAAGCAGGGGTTTAAAATGAATCCAAATCAGAAAATAATA
ACCATTGGATCAATCTGTCTGGTAGTCGGACTAATTAGCCTAAT
ATTGCAAATAGGGAATATAATCTCAATATGGATTAGCCATTCAA
TTCAAACTGGAAGTCAAAACCATACTGGAATATGCAACCAAAAC
ATCATTACCTATAAAAATAGCACCTGGGTAAAGGACACAACTTC
AGTGATATTAACCGGCAATTCATCTCTTTGTCCCATCCGTGGGT
GGGCTATATACAGCAAAGACAATAGCATAAGAATTGGTTCCAAA
GGAGACGTTTTTGTCATAAGAGAGCCCTTTATTTCATGTTCTCA
CTTGGAATGCAGGACCTTTTTTCTGACCCAAGGTGCCTTACTGA
ATGACAAGCATTCAAGTGGGACTGTTAAGGACAGAAGCCCTTAT
AGGGCCTTAATGAGCTGCCCTGTCGGTGAAGCTCCGTCCCCGTA
CAATTCAAGATTTGAATCGGTTGCTTGGTCAGCAAGTGCATGTC
ATGATGGCATGGGCTGGCTAACAATCGGAATTTCAGGTCCAGAT
AATGGAGCAGTGGCTGTATTAAAATACAACGGCATAATAACTGA
AACCATAAAAAGTTGGAGGAAGAAAATATTGAGGACACAAGAGT
CTGAATGTGCCTGTGTAAATGGTTCATGTTTTACTATAATGACT
GATGGCCCGAGTGATGGGCTGGCCTCGTACAAAATTTTCAAGAT
CGAAAAGGGGAAGGTTACTAAATCAATAGAGTTGAATGCACCTA
ATTCTCACTATGAGGAATGTTCCTGTTACCCTGATACCGGCAAA
GTGATGTGTGTGTGCAGAGACAATTGGCATGGTTCGAACCGGCC
ATGGGTGTCTTTCGATCAAAACCTGGATTATCAAATAGGATACA
TCTGCAGTGGGGTTTTCGGTGACAACCCGCGTCCCGAAGATGGA
ACAGGCAGCTGTGGTCCAGTGTATGTTGATGGAGCAAACGGAGT
AAAGGGATTTTCATATAGGTATGGTAATGGTGTTTGGATAGGAA
GGACCAAAAGTCACAGTTCCAGACATGGGTTTGAGATGATTTGG
GATCCTAATGGATGGACAGAGACTGATAGTAAGTTCTCTGTGAG
GCAAGATGTTGTGGCAATGACTGATTGGTCAGGGTATAGCGGAA
GTTTCGTTCAACATCCTGAGCTGACAGGGCTAGACTGTATGAGG
CCGTGCTTCTGGGTTGAATTAATCAGGGGACGACCTAAAGAAAA
AACAATCTGGACTAGTGCGAGCAGCATTTCTTTTTGTGGCGTGA
ATAGTGATACTGTAGATTGGTCTTGGCCAGACGGTGCTGAGTTG
CCATTCAGCATTGACAAGTAGTCTGTTCAAAAAACTCCTTGTTT
CTACT

High-titer A/PR/8/34 (H1N1, PR8(UW)) virus grows 10 times better than other A/PR/8/34 PR8 strains in eggs (10¹⁰ EID₅₀/mL; HA titer:1:8,000). Thus, replacement of the HA and NA genes of PR8(UW) with those of a currently circulating strain of influenza virus results in a vaccine strain that can be safely produced and validates the use of PR8(UW) as a master vaccine strain.

Genes that contribute to different growth properties between PR8(UW) and PR8 (Cambridge), which provides the non-HA and -NA genes of the N1BRG-14 vaccine strain, were determined. Higher titers in eggs were obtained when the majority of internal genes were from PR8(UW). Highest titers were with the M viral segment of PR8(UW) and the NS gene of PR8 (Cambridge). The NS gene in PR8(UW) has a K (lysine) at residue 55 while the NS gene in PR8(Cam) has a E (glutamic acid). The polymerase subunit (PA, PB1, and PB2) and NP genes of PR8(UW) enhanced the growth of an H5N1 vaccine seed virus in chicken embryonated eggs, and the NS gene of PR8(Cambridge) enhanced the growth of an H5N1 vaccine seed virus in chicken embryonated eggs. A tyrosine (Y) at position 360 in PB2 of PR8(UW) likely contributes to the high growth rate of that virus in MDCK cells.

### High Growth Viruses

To develop an high-yield A/PR/8/34 (HIN1; PR8) virus backbone for growth of vaccine virus in specific host cells, random mutagenesis of the internal genes of PR8UW was conducted. Random mutations were introduced into the UW-PR8 internal genes by error-prone PCR, after which plasmid libraries were prepared that possessed the random mutations in an individual UW-PR8 internal gene. Then virus libraries (PR8/H5N1) were generated that possessed random mutations in an individual UW-PR8 internal gene, along with the other wild type internal genes and the NA and ‘detoxified’ HA genes of A/chicken/Indonesia/NC/09 (H5N1) virus (Table B), to generate “6+2” recombinant viruses. Consecutive passages of the virus in MDCK cells were employed to select for variants with high-growth properties.

TABLE B
Virus libraries generated

| | | | | Titer of |
|---|---|---|---|---|
| | Internal genes | | | virus |
| | | Other internal | | library |
| Number | Gene library | genes | HA + NA | (pfu/ml) |
| Control | PR8 wild type | | NC/09/H5N1 | 3 × 10⁶ |
| 1 | PB2 | 5 UW-PR8 genes | NC/09/H5N1 | 2.1 × 10² |
| 2 | PB1 | 5 UW-PR8 genes | NC/09/H5N1 | 1.6 × 10⁵ |
| 3 | PA | 5 UW-PR8 genes | NC/09/H5N1 | 7 × 10³ |
| 4 | NP | 5 UW-PR8 genes | NC/09/H5N1 | 1.5 × 10³ |
| 5 | M | 5 UW-PR8 genes | NC/09/H5N1 | 1 × 10⁶ |
| 6 | NS | 5 UW-PR8 genes | NC/09/H5N1 | 1.8 × 10⁶ |
| 7 | PB2 + PB1 + PA | 3 UW-PR8 genes | NC/09/H5N1 | 75 |
| 8 | PB2 + PB1 + | 2 UW-PR8 genes | NC/09/H5N1 | 33 |
| | PA + NP | | | |
| 9 | PB2 + NS | 4 UW-PR8 genes | NC/09/H5N1 | 2 × 10² |
| 10 | M + NS | 4 UW-PR8 genes | NC/09/H5N1 | 5.7 × 10⁵ |

Virus libraries were passaged 12 times in MDCK cells or, after 2 passages, the libraries were mixed and 10 more passages were carried out.

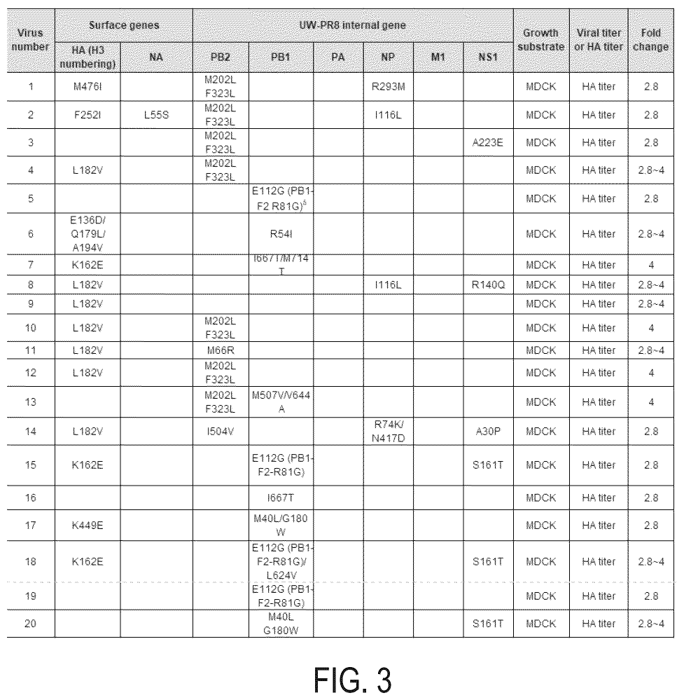

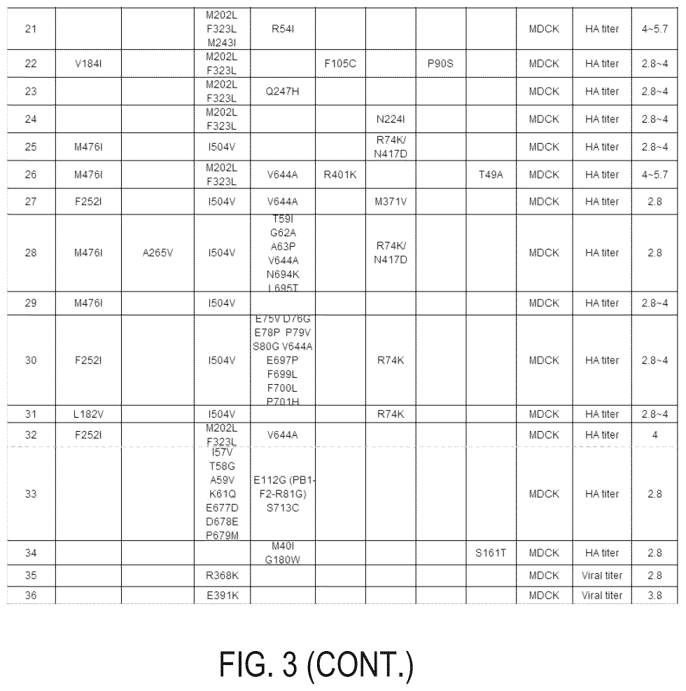

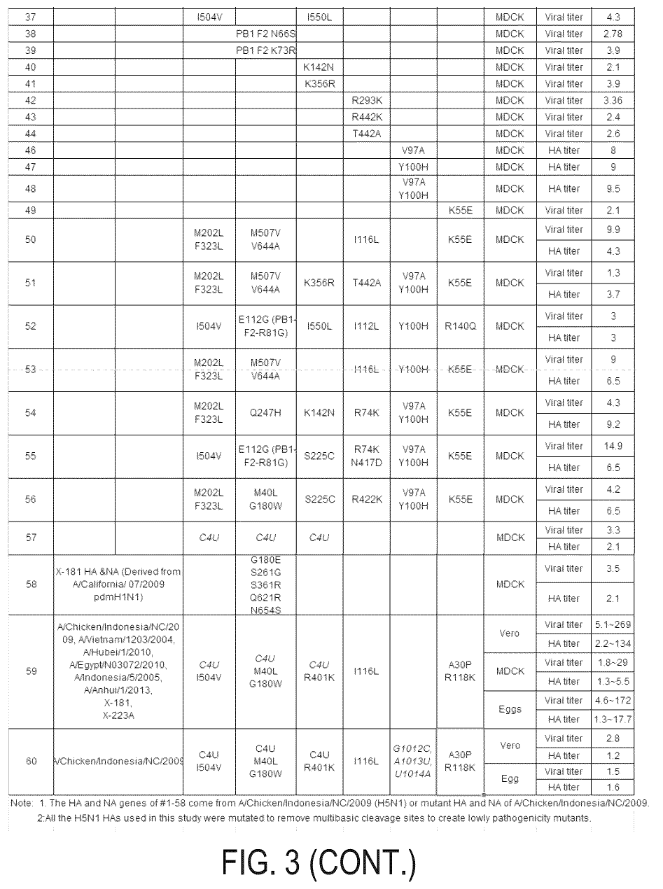

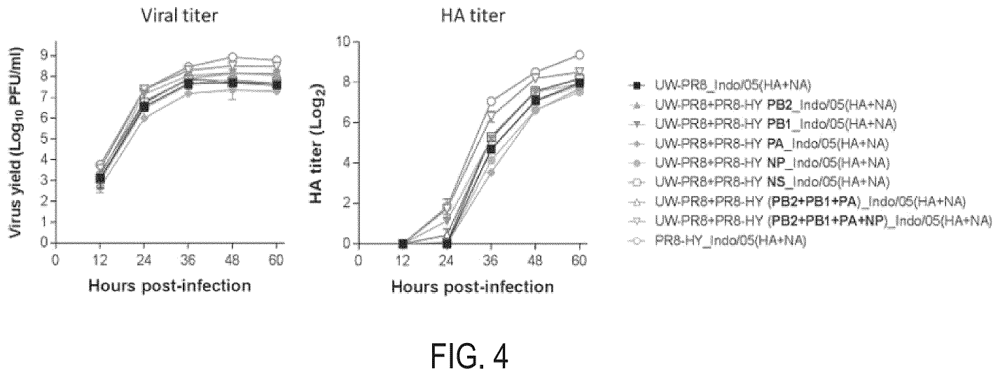

After 10 to about 12 consecutive passages in MDCK cells, plaque assays were performed and over 1,400 individual plaques were picked. FIG. 3 shows the numbers of clones with various HA titers. Growth-enhancing mutations included: PB2: M202L, F323L, 1504V, PBL: E1112G, V644A, NP: R74K, N4171D, I116L, and NS: S161T.

36 viruses with the highest HA titers from the random mutagenesis libraries were sequenced (Table C).

TABLE C
Sequences of viruses with the highest HA titers

| | | HA | | | | | | | | |
|---|---|---|---|---|---|---|---|---|---|---|
| Clone | | titer | | | | HA (H3 | | | | |
| # | Library | (2ⁿ) | PB2 | PB1 | PA | numbering) | NP | NA | M | NS |
| WT | | 7 | | | | | | | | |
| 329 | Mix | 9 | M202L | | | L182V | | | | |
| | | | F323L | | | | | | | |
| 154 | Mix | 8.5~9 | M202L | | | L182V | | | | |
| | | | F323L | | | | | | | |
| 347 | Mix | 9 | M202L | | | L182V | | | | |
| | | | F323L | | | | | | | |
| 94 | Mix | 8.5 | M202L | | | F252I | I116L | L55S | | |
| | | | F323L | | | | | | | |
| 1045 | Mix | 9 | M202L | V644A | | F252I | | | | |
| | | | F323L | | | | | | | |
| 965 | Mix | 8.5~9 | M202L | | F105C | V184I | | | P90S | |
| | | | F323L | | | | | | | |
| 50 | Mix | 8.5 | M202L | | | M148I | R293M | | | |
| | | | F323L | | | (HA2) | | | | |
| 1005 | Mix | 9~9.5 | M202L | V644A | R401K | M148I | | | | T49A |
| | | | F323L | | | (HA2) | | | | |
| 134 | Mix | 8.5 | M202L | | | | | | | A223E |
| | | | F323L | | | | | | | |
| 387 | Mix | 9 | M202L | M507V | | | | | | |
| | | | F323L | V644A | | | | | | |
| 852 | Mix | 9~9.5 | M202L | R54I | | | | | | |
| | | | F323L | | | | | | | |
| | | | M243I | | | | | | | |
| 981 | Mix | 8.5~9 | M202L | Q247H | | | | | | |
| | | | F323L | | | | | | | |
| 993 | Mix | 8.5~9 | M202L | | | | N224I | | | |
| | | | F323L | | | | | | | |
| 1043 | Mix | 8.5~9 | I504V | | | L182V | R74K | | | |
| 398 | Mix | | I504V | | | L182V | R74K, | | | A30P |
| | | | | | | | N417D | | | |
| 1007 | Mix | 8.5 | I504V | V644A | | F252I | M371V | | | |
| 1042 | Mix | 8.5~9 | I504V | E75V | | F252 | R74K | | | |
| | | | | D76G | | | | | | |
| | | | | E78P | | | | | | |
| | | | | P79V | | | | | | |
| | | | | S80G | | | | | | |
| | | | | V644A | | | | | | |
| | | | | E697P | | | | | | |
| | | | | F699L | | | | | | |
| | | | | F700L | | | | | | |
| | | | | P701H | | | | | | |
| | | | | S702R | | | | | | |
| | | | | Y705T | | | | | | |
| 999 | Mix | 8.5~9 | I504V | | | M148I | R74K, | | | |
| | | | | | | (HA2) | N417D | | | |
| 1014 | Mix | 8.5 | I504V | T59I | | M148I | R74K, | A265V | | |
| | | | | G62A | | (HA2) | N417D | | | |
| | | | | A63P | | | | | | |
| | | | | V644A | | | | | | |
| | | | | N694K | | | | | | |
| | | | | L695T | | | | | | |
| 1016 | Mix | 8.5~9 | I504V | | | M148I | | | | |
| | | | | | | (HA2) | | | | |
| 540 | PB1 | 8.5 | | E112G | | K162E | | | | S161T |
| | | | | (PB1-F2- | | | | | | |
| | | | | R81G) | | | | | | |
| 548 | PB1 | 8.5~9 | | E112G | | K162E | | | | S161T |
| | | | | (PB1-F2- | | | | | | |
| | | | | R81G) | | | | | | |
| | | | | L624V | | | | | | |
| 191 | PB1 | 8~8.5 | | E112G | | | | | | |
| | | | | (PB1-F2- | | | | | | |
| | | | | R81G) | | | | | | |
| 571 | PB1 | 9~9.5 | | E112G | | | | | | |
| | | | | (PB1-F2- | | | | | | |
| | | | | R81G) | | | | | | |
| 572 | PB1 | 8.5 | | E112G | | | | | | |
| | | | | (PB1-F2- | | | | | | |
| | | | | R81G) | | | | | | |
| 573 | PB1 | 8.5 | | E112G | | | | | | |
| | | | | (PB1-F2- | | | | | | |
| | | | | R81G) | | | | | | |
| 1404 | PB1 | 8.5 | I57V | E112G | | | | | | |
| | | | T58G | (PB1-F2- | | | | | | |
| | | | A59V | R81G) | | | | | | |
| | | | K61Q | S713C | | | | | | |
| | | | E677D | | | | | | | |
| | | | D678E | | | | | | | |
| | | | P679M | | | | | | | |
| 1408 | PB1 | 8.5 | | M40I | | | | | | S161T |
| | | | | G180W | | | | | | |
| 582 | PB1 | 8.5~9 | | M40L, | | | | | | S161T |
| | | | | G180W | | | | | | |
| 545 | PB1 | 8.5 | | M40L, | | K121E | | | | |
| | | | | G180W | | (HA2) | | | | |
| 543 | PB1 | 8.5 | | I667T | | | | | | |
| 219 | PB1 | 9 | | I667T, | | K162E | | | | |
| | | | | M714T | | | | | | |
| 344 | Mix | 8.5~9 | M66R | | | L182V | | | | |
| 312 | Mix | 8.5~9 | | | | L182V | I116L | | | R140Q |
| 320 | Mix | 8.5 | | | | L182V | | | | |
| 209 | PB1 | 8.5~9 | | R54I | | E136D, | | | | |
| | | | | | | Q179L, | | | | |
| | | | | | | A194V | | | | |

In a second approach, potentially growth-enhancing mutations described in the literature were introduced into the background of UW-PR8 virus (see Table C for virus stock titers) and tested for replicative ability.

TABLE D
UW-PR8 viruses possessing mutation(s) identified in the literature

| | | Virus stock titer |
|---|---|---|
| Gene | Mutation(s) | (Pfu/ml) |
| WT | — | 2 × 10⁷ |
| PB2 | A44S | 4.5 × 10⁷ |
| | E158G | 3.2 × 10⁴ |
| | E158G + NP N101G | 7.5 × 10⁴ |
| | E158A | 8.3 × 10⁶ |
| | D253N + Q591K | 8.3 × 10⁶ |
| | D256G | 2.8 × 10⁷ |
| | R368K | 3.1 × 10⁷ |
| | E391Q | 1.4 × 10⁸ |
| | I504V + PA I550L | 1.1 × 10⁸ |
| | Q591K | 4.4 × 10⁷ |
| | V613T | 1.8 × 10⁷ |
| | A661T | 2.2 × 10⁷ |
| | D701N + S714R + NP N319K | 1 × 10⁶ |
| | D701N | 2.1 × 10⁷ |
| PB1 | R327K | 1.3 × 10⁷ |
| | V336I | 2.3 × 10⁷ |
| | L473V + L598P | 3.9 × 10⁵ |
| PB1F2 | F2 N66S | 1.6 × 10⁸ |
| | F2 K73R | 1.1 × 10⁸ |
| | F2 V76A | 4.4 × 10⁷ |
| | F2 R79Q | 6.2 × 10⁶ |
| | F2 L82S | 2.7 × 10⁷ |
| | F2 E87Q | 1.5 × 10⁶ |
| PA | T97I | 1.6 × 10⁷ |
| | K142N | 3.3 × 10⁷ |
| | S225C | 6.7 × 10⁷ |
| | S149P + T357K | 3.4 × 10⁸ |
| | K356R | 8.5 × 10⁷ |
| | A404S | 5.2 × 10⁷ |
| | S421I | 2.7 × 10⁷ |
| NP | R293K | 4.7 × 10⁷ |
| | R305K | 7.2 × 10⁷ |
| | E372D | 2.2 × 10⁷ |
| | R422K | 1.3 × 10⁸ |
| | T442A | 5 × 10⁷ |
| | D455E | 2.2 × 10⁷ |
| | I109V | 3.9 × 10⁷ |
| M | V97A + Y100H | 1.4 × 10⁷ |
| NS1 | K55E | 1.6 × 10⁷ |

In a third approach, candidates from approaches 1 and 2 were combined and HA titers and PFU/mL determined (Table E).

TABLE E
High-growth candidates identified in approaches 1 and 2 were tested in various combinations.

| | Gene origin | | | | | | | | Virus stock titer | |
|---|---|---|---|---|---|---|---|---|---|---|
| # | HA | NA | PB2 | PB1 | PA | NP | M | NS | HA (2ⁿ) | Pfu/ml |
| WT | Indo/NC/09 | Indo/NC/09 | UW- | UW- | UW- | UW- | UW- | UW- | 7 | 3.00E+07 |
| | (detoxified) | | PR8 | PR8 | PR8 | PR8 | PR8 | PR8 | | |
| 1 | | | M202L | M507V | | I116L | | K55E | 9~9.5 | 2.00E+08 |
| | | | F323L | V644A | | | | | | |
| 2 | | | M202L | R54I | | N224I | | K55E | 5 | 1.00E+05 |
| | | | F323L | | | | | | | |
| 3 | | | M202L | Q247H | R401K | | | T49A | 9 | 1.00E+08 |
| | | | F323L | | | | | | | |
| 4 | | | M202L | M507V | K356R | T442A | V97A | K55E | 10~10.5 | 1.60E+08 |
| | | | F323L | V644A | | | Y100H | | | |
| 5 | | | I504V | M507V | I550L | R74K | | K55E | 8~8.5 | 5.70E+07 |
| | | | | V644A | | N417D | | | | |
| 6 | | | I504V | M507V | I550L | R74K | V97A | K55E | 9~9.5 | 4.40E+07 |
| | | | | V644A | | N417D | Y100H | | | |
| 7 | | | I505V | E112G | I550L | R74K | | S161T | 9 | 1.60E+08 |
| 8 | | | M202L | I667T | | I116L | | R140Q | <1 | <1E3 |
| | | | F323L | M714T | | | | | | |
| 9 | | | M202L | E112G | | | | S161T | 8.5 | 1.30E+08 |
| | | | F323L | (PB1-F2- | | | | | | |
| | | | | R81G) | | | | | | |
| 10 | | | M66R | M40I | | R74K | | S161T | 8~8.5 | 2.30E+07 |
| | | | | G180W | | | | | | |
| 12 | | | R368K | PB1 F2 | K356R | R422K | | K55E | 5.5 | 9.00E+02 |
| | | | | N66S | | | | | | |
| 13 | | | E391Q | R327K | S149P | R293K | | | 3 | 1.60E+06 |
| | | | | | T357K | | | | | |
| 14 | | | Q591K | PB1 F2 | S225C | R422K | | K55E | 7.5 | 2.00E+07 |
| | | | | K73R | | | | | | |
| 23 | | | | | | | V97A | | 8.5~9 | 1.50E+07 |
| 24 | | | | | | | Y100H | | 9~9.5 | 2.90E+07 |
| 25 | NCR 15- | Indo/NC/09 | M202L | M507V | K356R | R422K | V97A | K55E | 9.5~10 | 7.50E+07 |
| | 19nt mut¹ | | F323L | V644A | | | Y100H | | | |
| 26 | Indo/NC/09 | Indo/NC/09 | | | | | | A30P | 6.5~7 | 1.00E+07 |
| 27 | (detoxified) | | | | | | | T49A | 6.5~7 | 2.00E+07 |
| 28 | | | | | | | | R140Q | 8 | 4.00E+07 |
| 29 | | | | | | | | S161T | 7~7.5 | 1.40E+07 |
| 30 | | | | | | | | A223E | 7.5 | 1.00E+07 |
| 31 | | | | I667T | | | | | 3.5 | 4.00E+05 |
| | | | | M714T | | | | | | |
| 32 | NCR 15- | UW-PR8 | M202L | V644A | K356R | T442A | Y100H | K55E | 7~7.5 | 4.30E+06 |
| | 19nt mut | | F323L | | | | | | | |
| 33 | Indo/NC/09 | Indo/NC/09 | M202L | E112G | K356R | R74K | Y100H | K55E | 9~9.5 | 7.00E+07 |
| | (detoxified) | | F323L | (PB1-F2- | | | | | | |
| | | | | R81G) | | | | | | |
| 34 | NCR 15- | UW-PR8 | I504V | M507V | | | V97A | K55E | 7 | 2.00E+05 |
| | 19nt mut | | | V644A | | | Y100H | | | |
| 35 | Indo/NC/09 | Indo/NC/09 | M202L | M507V | R401K | T442A | Y100H | R140Q | 9 | 3.20E+07 |
| | (detoxified) | | F323L | V644A | | | | | | |
| 36 | | | I504V | E112G | I550L | I112L | Y100H | R140Q | 9.5 | 1.30E+08 |
| | | | | (PB1-F2- | | | | | | |
| | | | | R81G) | | | | | | |
| 37 | | | M202L | E112G | S149P | T442A | Y100H | K55E | 0 | 0.00E+00 |
| | | | F323L | (PB1-F2- | T357K | | | | | |
| | | | | R81G) | | | | | | |
| 38 | | | M202L | M507V | | I116L | Y100H | K55E | 10.1 | 2.30E+08 |
| | | | F323L | V644A | | | | | | |
| 39 | | | M202L | M507V | K356R | T442A | Y100H | K55E | 9.8 | 1.00E+08 |
| | | | F323L | V644A | | | | | | |
| 40 | | | I504V | M507V | I550L | T442A | Y100H | K55E | 9.2 | 6.00E+07 |
| | | | | V644A | | | | | | |
| 41 | | | I504V | I112E | I550L | R74K | Y100H | K55E | 9.2 | 7.50E+07 |
| P17 | | | I504V | E112G | S225C | R74K | V97A | K55E | 9.5~10 | 5.80E+08 |
| | | | | (PB1-F2- | | N417D | Y100H | | | |
| | | | | R81G) | | | | | | |
| P26 | | | M202L | M40L | S225C | R422K | V97A | K55E | 10 | 3.00E+08 |
| | | | F323L | G180W | | | Y100H | | | |
| P61 | | Indo/NC/09 | M202L | Q247H | K142N | R74K | V97A | K55E | 10~10.5 | 2.00E+08 |
| | | NA P263T² | F323L | | | | Y100H | | | |

¹Mutation in the HA gene noncoding region;
²A P263T mutation was detected in the NA protein of this virus clone

As shown in Table E, several recombinant viruses were identified that replicated better than wild type, such as #1, #4, #36, #38, P17, P16, and P61. To identify the growth characteristics of these viruses, growth kinetics in MDCK cells were determined. For one candidate, virus was purified on sucrose gradients and HA content and viral total protein evaluated. Further analysis demonstrated that viruses possessing the V97A/Y100H mutations in M1 yielded higher HA titers than the parental virus, although the virus titer was lower. The V97A/Y100H mutations in M1 may result in particles with a larger surface into which more HA protein can be incorporated. Since inactivated influenza viruses are dosed based on their HA content, variants with high HA content are attractive vaccine candidates.

To identify mutations in the influenza promoter region that provide for enhanced replication, viruses possessing a ‘U’ at position 4 at the 3′ end of all eight vRNA segments were prepared in the UW-PR8 PA, PB1 and PB2 internal genes (the UW-PR8 PB2, PB, and PA segments possess a ‘C’ at position 4).

Viruses possessing combinations of promoter mutations and amino acid changes were prepared and titers determined (Table F).

TABLE F
Virus titers of high-growth candidates.

| | Gene backbone | | | | | | | | Virus stock titer | |
|---|---|---|---|---|---|---|---|---|---|---|
| Viruses | HA | NA | PB2 | PB1 | PA | NP | M | NS | HA (2ⁿ) | pfu/ml |
| Control | WT | WT | WT | WT | WT | WT | WT | WT | 7 | 3.0E+07 |
| 1 | WT | WT | 3′C4U | 3′C4U | 3′C4U | R74K | V97A | K55E | 10.5 | 2.2E+09 |
| 2 | 3′ G3A U5C C8U & | | M202L | Q247H | K142N | | Y100H | | 8.5~9 | 5.6E+07 |
| | 5′ U3C A8G | | F323L | | | | | | | |
| 3 | NCR 15-19nt mut | | | | | | | | 9~9.5 | 1.4E+09 |
| 4 | 3′ G3A U5C C8U & | | | | | | | | 7 | 7.0E+07 |
| | 5′ U3C A8G & NCR | | | | | | | | | |
| | 15-19nt mut | | | | | | | | | |

Optimization of rare codons in PB2 resulted in increased titers compared to wild type virus (UJW-PR8). Other viral segments were codon optimized and titers of viruses with those segments or combinations of optimized segments were determined.

In another approach to increase virus titer in MDCK cells, chimeric HA and NA genes were prepared and titers of viruses having those genes were determined.

Viruses with combinations of the above-mentioned mutations (high growth backbone mutations, promoter mutations, chimeric HA and NA genes and canine codon optimization) were prepared and growth kinetics, PFU and HA titers of those viruses were determined. An example set of backbone mutations are canine codon opti-PB2+C4U+M202L, F323L; PB 1: C4U+Q247H; PA: C4U+K142N; NP: Canine codon opti-NP+R74K; M. V97A, Y100H; and NS: K55E.

Any of the mutations described herein, or any combination thereof, may be combined with, for instance, seasonal HIN1 and H3N2, H3N2 Variant, PdmH1N1, H5N1, H7N9 or H9N2, or other clades or candidate vaccine strains. For example, HA and NA genes from A/California/04/2009(pdm H1N1) were combined with the six internal genes of UW-PR/8 to generate “6+2” recombinant viruses. Eleven virus libraries were generated and passaged 10 times in eggs. Three rounds of limiting dilution were performed to screen for high growth mutants. In embodiments, a variant with high growth properties in MDCK cells has a PB2 viral segment with a promoter mutation (C4U) and a mutation that results in I504V (relative to the parental virus), a PB1 viral segment with a promoter mutation (C4U) and a mutation that results in E112G; a PA viral segment with a promoter mutation (C4U) and a mutation that results in S225C; a NP viral segment with mutations that result in R74K and N417D: a M viral segment with mutations that result in V97A and Y100H; and a NS viral segment with a mutation that results in K55E, where optionally the sequence of one or more viral segments, e.g., the NP viral segment, is modified to include canine codon optimized codons. In embodiments, a variant with high growth properties in MDCK cells has a canine codon optimized PB2 viral segment with a promoter mutation (C4U) and mutations that result in M202L and F323L; a PB1 viral segment with a promoter mutation (C4U) and a mutation that results in Q247H; a PA viral segment with a promoter mutation (C4U) and a mutation that results in K142N; a canine codon optimized NP viral segment with a mutation that results in R74K: a M viral segment with mutations that result in V97AY100H; and a NS viral segment with a mutation that results in K55E.

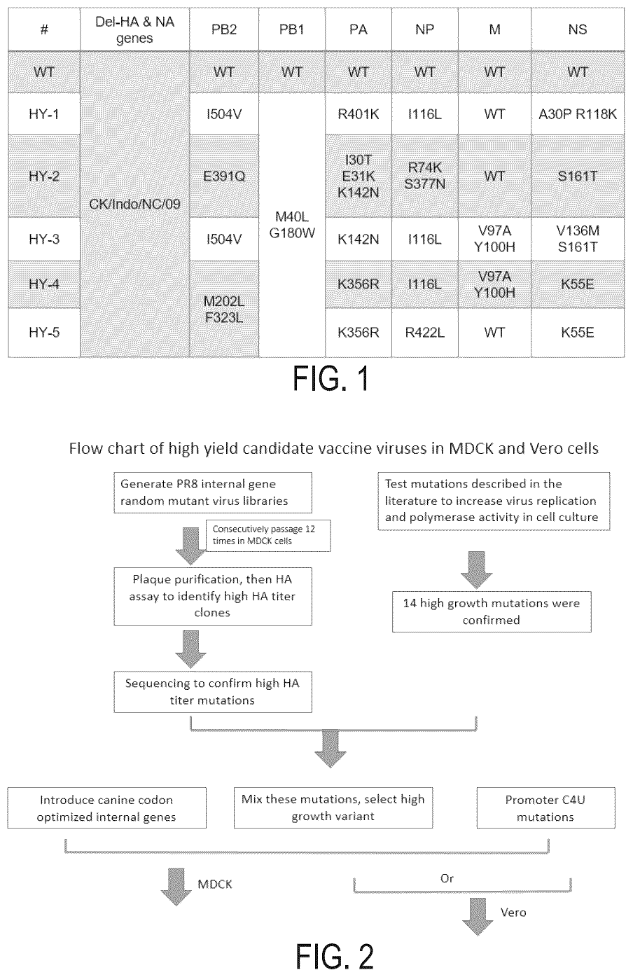

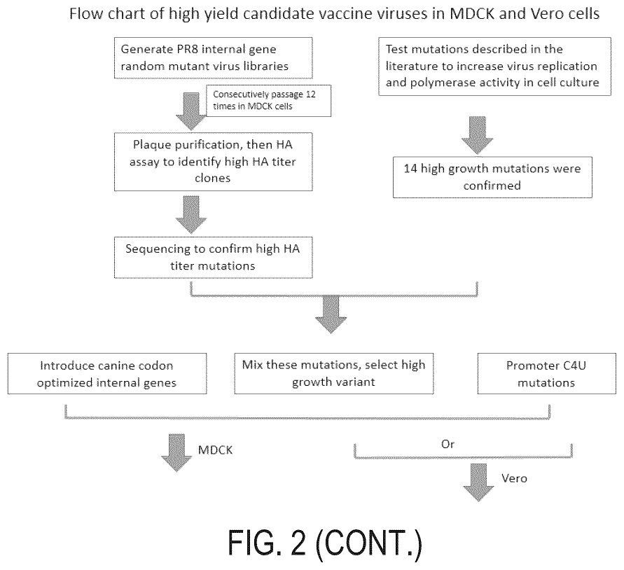

Similar experiments were conducted in Vero cells, e.g., after about 3 to 5 passages in Vero cells, using clones with high replicative properties in MDCK cells. FIG. 1 shows 5 viruses likely to have high replicative properties in Vero cells. In embodiments, a PR8(UW) variant with high-growth properties in Vero cells has the following mutations that may be used in various combinations to increase the replicative ability of PR8(UW) virus: PB2 segment: C4U (promoter mutation), I504V (amino acid change); PB1 segment: C4U (promoter mutation); M40L (amino acid change), G180W (amino acid change); PA segment: C4U (promoter mutation), R401K (amino acid change); NP segment: I116L (amino acid change); NS segment: A30P (amino acid change in NS1), or R118K (amino acid change in NS1).

In embodiments, a PR8(UW) variant with high-growth properties has the following residues that may be used in various combinations with each other and/or other residues, e.g., those that enhance virus replication, to increase the replicative ability of reassortants having PR8(UW) based viral segment(s): a HA segment with one or more of 136D, 162E, 179L, 182V, 184I, 2521, 449E, and/or 476I: a NA segment with 55S and/or 265V; a NS segment with NS1 having 118K; F2 with 81G; a PB1 segment with 62A, 261G, 361R, 621R, and/or 654S, and/or viral segment promoters with the growth-enhancing nucleotides described herein, e.g., having one or more of the nucleotide changes G1012C, A1013U, or U1014A in the M viral segment.

To assess the contribution of individual viral RNA (vRNA) segments to high-yield properties, a series of reassortant viruses was generated that possessed one or several vRNA segments of a high-yield PR8 (PR8-HY) variant in the background of the parental virus [UW-PR8_indo/05(HA+NA)]. Vero cells were infected in triplicate with the indicated viruses at a MOI of 0.005 and incubated at 37° C. in the presence of trypsin. At the indicated time points, virus titers and HA titers were determined by performing plaque or HA assays, respectively. The results are shown in FIG. 20. These data indicated that several vRNA segments contribute to the properties of PR8-HY virus. In particular, the PB2+PB1+PA+NP vRNAs of PR8-HY virus conferred an appreciable increase in virus and HA titers, evidencing the enhanced replicative ability of this virus.

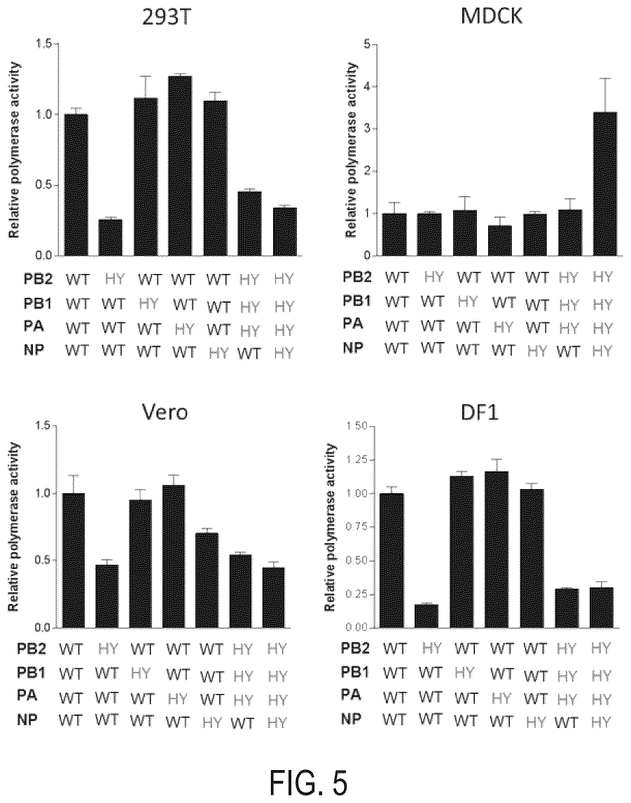

To further assess which component of the viral replication complex that provides for high-yield properties, wild-type or high-yield PB2, PB1, PA, and NP proteins were tested in various combinations in minireplicon assays in human 293T, canine MDCK, African green monkey Vero, and avian DF1 cells. The results are shown in FIG. 5. Interestingly, the PB2, PB1, PA, and NP proteins of PR8-HY virus attenuated the viral replicative ability in 293T, Vero, and DF1 cells; this effect was primarily conferred by the PB2 protein. In contrast, the combination of PB2+PB1+PA+NP proteins derived from PR8-HY virus conferred a substantial increase in replicated ability in canine MDCK cells, which were used for the selection of PR8-HY virus. The findings suggested host-dependent mechanisms underlying the high yield of PR8-HY virus. For example, the combination of PB1+PA+NP proteins, or a subset thereof, derived from PR8-HY may confer enhanced viral replicative ability in 293T, Vero, and DF1 cells.

Libraries were screened in serum-free (SF) medium, serum-containing (SC) medium, and eggs. To identify a variant that replicates efficiently in all growth media tested, viruses were passaged under changing conditions:

SF to SC to Eggs, SF to Eggs to SC
SC to SF to Eggs, SC to Eggs to SF
Eggs to SF to SC, Eggs to SC to SF

For example, other identified mutations that further improve the replication of influenza virus in cultured cells and/or embryonated chicken eggs are I711V in PB1 and M128L in the M1 protein, and those may be combined with any of the mutations disclosed herein, and in any combination (see Tables G-H). The viral titers for HY candidate #9 were 5.5-fold, 2.4-fold, 2.6-fold, 4.4-fold higher than those of the parental high yield backbone recombinant virus at 12h, 24h, 36h and 48h. HY candidate #9 contains the following mutations: PB2: C4U I504V; PB1: C4UM40L/G180W/1711V; PA: C4U R401K; NP: I116L; M1: M128L; NS1: A30P/R118K.

TABLE G
Top 8 high HA titer clones selected from SF-MDCK PR8-HY + Hubei/1/10(H5N1) virus libraries.

| | Virus stock titer | | Mutations | | | | | | | |
|---|---|---|---|---|---|---|---|---|---|---|
| | HA titer | | | | | | | | HA (H3 | |
| Virus | (2\hat{ }n) | PFU/ml | PB2 | PB1 | PA | NP | M1 | NS1 | numbering) | NA |

| | | | | | | | | | |
|---|---|---|---|---|---|---|---|---|---|
| WT | — | — | C4U | C4U | C4U | I116L | | A30P/ | |
| | | | I504V | M40L/ | R401K | | | R118K | |
| | | | | G180W | | | | | |
| SF-14 | 10 | 1.44 × 10\hat{ }9 | | +I711V | | | +M128L | | K216N |
| SF-22 | 9.4 | 6.6 × 10\hat{ }8 | | +I711V | | | | | K216N |
| SF-37 | 9.6 | 8.7 × 10\hat{ }8 | | +I711V | | | | | K216N |
| SF-46 | 9.3 | 8.3 × 10\hat{ }8 | +M467I/ | | | | | | K222T/ |
| | | | K721R | | | | | | G326R |
| SF-42 | 9.5 | 8.7 × 10\hat{ }8 | | +I711V | +R256G | | | | K216N |
| SF-57 | 9.6 | 1 × 10\hat{ }9 | | | | | | | K222T |
| SF#15 | 10 | 1.24 × 10\hat{ }9 | +Y704H | | | | | | K222T |
| SF-E-85 | 9.6 | 9 × 10\hat{ }8 | | | +N350I/ | | | | K222T |
| | | | | | A369T | | | | |

TABLE H
Top 12 HA titer clones selected from one round of passage of second-generation mixed library (SC = serum containing; SF = serum-free).

| | | HA | | | | | | | | | |
|---|---|---|---|---|---|---|---|---|---|---|---|
| MEDI- | PLAQUE | TITER | | | | | | | | | |
| UM | # | (2\hat{ }N) | HA&NA | PB2 | PB1 | PA | NP | M1 | M2 | NS1 | NS2 |
| | WT | | DETOXIFIED- | C4U | C4U | C4U | I116L | | | A30P/ | |
| | | | A/HUBEI/1/ | I504V | M40L/ | R401K | | | | R118K | |
| | | | 2010 | | G180W | | | | | | |
| SC | 11 | 7.5 | (HA + NA) | C4UI504V + | C4U | C4U | I116L + | S126G + | W15G | A30P/ | I76M |
| ↓ | | | | A221V | M40L/ | R401K | K103R + | 244 | | R118K + | |
| EGG | | | | | G180W | | V194I | (MIXTURE) | | D101V | |
| ↓ | | | | | | | | M/R | | | |
| SF | 14 | 7.5 | | C4UI504V + | C4U | C4U | I116L + | M128L + | W15G | A30P/ | E82D |
| | | | | A221V | M40L/ | R401K | K103R + | 244 | | R118K | |
| | | | | | G180W + | | V194I | (MIXTURE) | | | |
| | | | | | I711V | | | M/R | | | |
| | 23 | 8.0 | | C4UI504V + | C4U | C4U | I116L + | F62L + | W15G | A30P/ | |
| | | | | 62S + | M40L/ | R401K | K103R + | 244 | | R118K | |
| | | | | A370T | G180W + | | V194I | (MIXTURE) | | | |
| | | | | | I711V | | | M/R | | | |
| | 27 | 7.5 | | C4UI504V + | C4U | C4U | I116L | S126G | W15G | A30P/ | |
| | | | | R62S | M40LG180W + | R401K | | | | R118K | |
| | | | | | E178K + | | | | | | |
| | | | | | M290I | | | | | | |
| | 33 | 7.5 | | C4U | C4U | C4U | I116L | S126G + | W15G | A30P/ | I76M |
| | | | | 1504V | M40L/ | R401K + | | 244 | | R118K + | |
| | | | | | G180W + | K142E | | (MIXTURE) | | D101V | |
| | | | | | I711V | | | M/R | | | |
| SC | 1 | 7.5 | | C4U | C4U | C4U | I116L | S126G + | W15G | A30P/ | N92D |
| ↓ | | | | I504V + | M40L/ | R401K + | | 244 | | R118K | |
| SF | | | | 62S + | G180W + | K256G | | (MIXTURE) | | | |
| ↓ | | | | A370T | E178K + | | | M/R | | | |
| EGG | | | | | M92I | | | | | | |
| | 2 | 7.5 | | C4UI504V + | C4U | C4U | I116L | M128L + | | A30P/ | I76M |
| | | | | A221V | M40L/ | R401K + | | 244 | | R118K + | |
| | | | | | G180W + | K256G | | (MIXTURE) | | D101V | |
| | | | | | M92I | | | M/R | | | |
| | 4 | 7.5 | | C4U | C4U | C4U | I116L | 244 | W15G | A30P/ | |
| | | | | I504V + | M40L/ | R401K + | | (MIXTURE) | | R118K | |
| | | | | R62S | G180W + | K256G | | M/R | | | |
| | | | | | M325V | | | | | | |
| | 5 | 8.0 | | C4UI504V + | C4U | C4U | I116L | S126G + | W15G | A30P/ | E82D |
| | | | | A221V | M40L/ | R401K + | | 244 | | R118K | |
| | | | | | G180W + | K142E | | (MIXTURE) | | | |
| | | | | | M92I | | | M/R | | | |
| | 13 | 8.0 | | C4UI504V | C4U | C4U | I116L + | M128L + | W15G | A30P/ | N92D |
| | | | | | M40L/ | R401K + | K103R + | 244 | | R118K | |
| | | | | | G180W + | K142E | V194I | (MIXTURE) | | | |
| | | | | | M92I | | | M/R | | | |
| | 24 | 8.0 | | C4UI504V + | C4U | C4U | I116L + | M128L + | W15G | A30P/ | E82D |
| | | | | R62S + | M40L/ | R401K + | K103R + | 244 | | R118K | |
| | | | | A370T | G180W | K142E | V194I | (MIXTURE) | | | |
| | | | | | | | | M/R | | | |
| | 25 | 7.5 | | C4U | C4U | C4U | I116L + | S126G + | W15G | A30P/ | N92D |
| | | | | 1504V | M40L/ | R401K | K103R + | 244 | | R118K | |
| | | | | | 180W + | | V194I | (MIXTURE) | | | |
| | | | | | M92I + | | | M/R | | | |
| | | | | | N105H | | | | | | |

The invention will be described by the following nonlimiting example.

### Example 1

### Materials and Methods

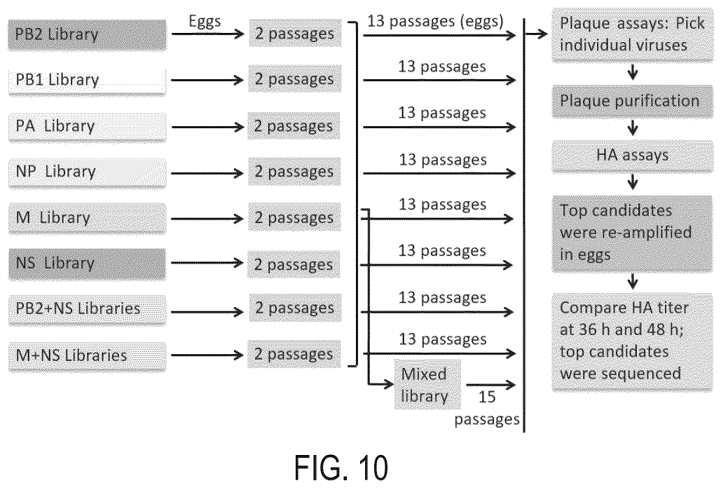

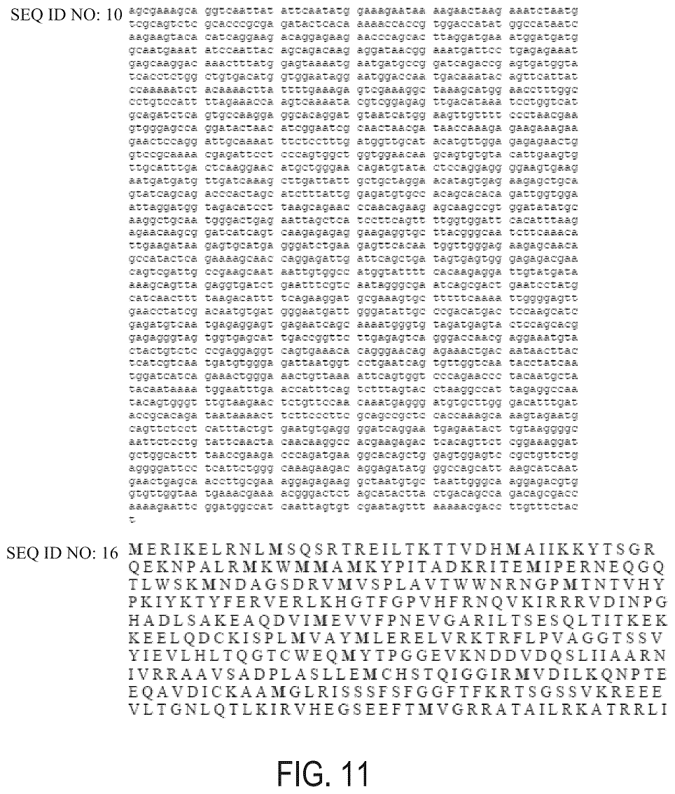

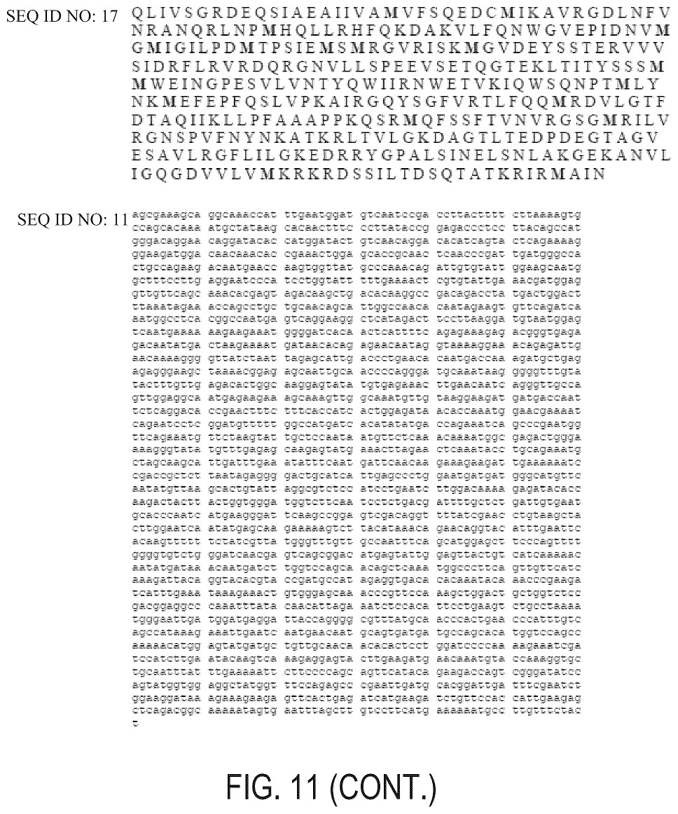

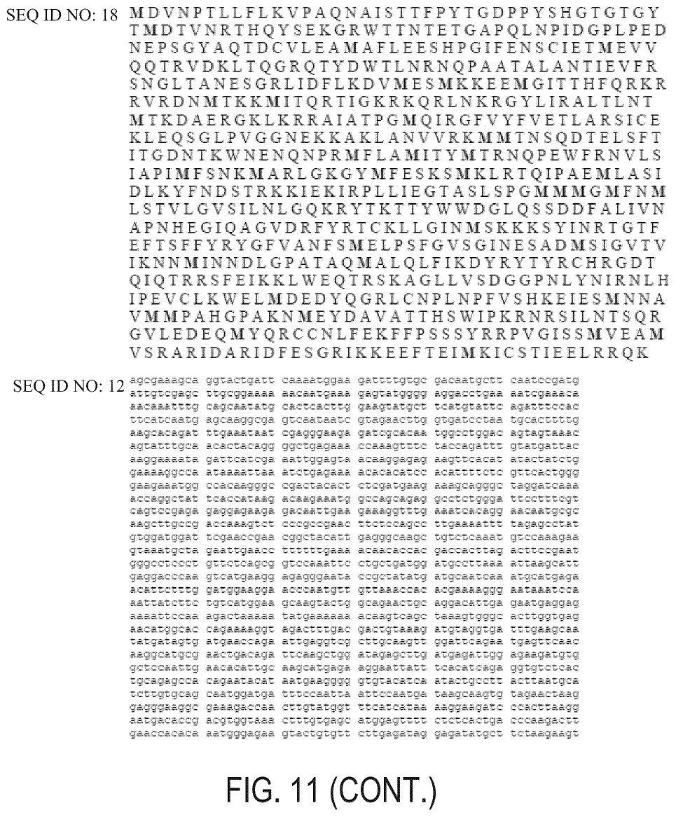

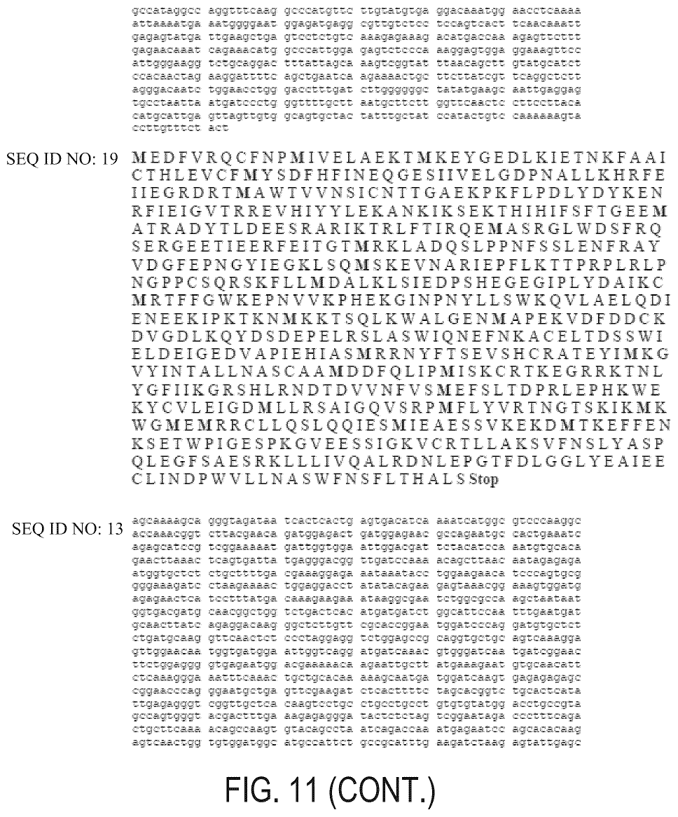

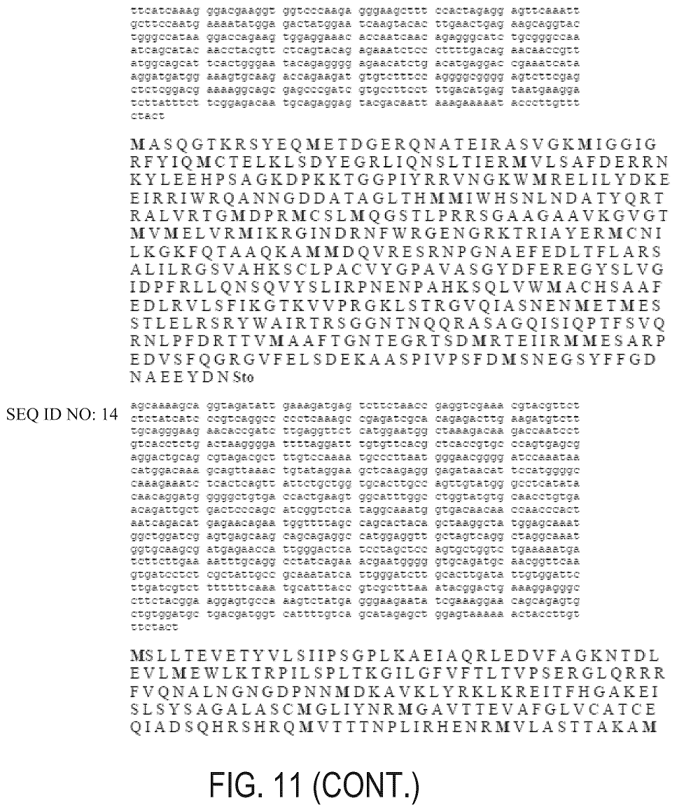

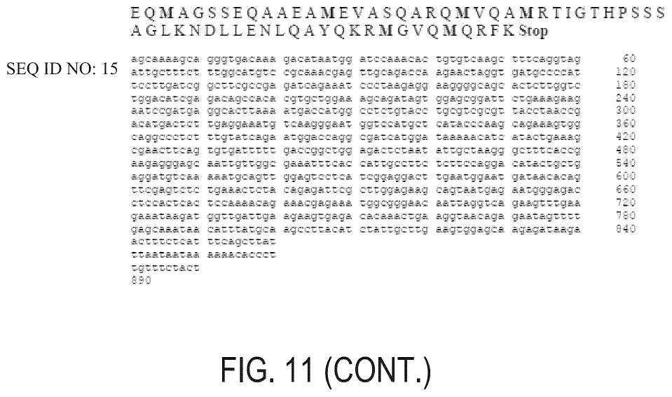

Virus and cells. 293T human embryonic kidney cells were maintained in DMEM supplemented with 10% fetal bovine serum (FBS). MDCK cells was grown in MEM containing 5% newborn calf serum (NCS). Vaccine viruses IVR-186 (derived from A/Singapore/INFIMH-16-0019/2016, i.e., a seasonal clade 3C.2a1 H3N2 virus), NIB-112 (derived from A/Switzerland/8060/2017, i.e., a seasonal clade 3C.2a2 H3N2 virus), NYMC X-327 (derived from A/Kansas/14/2017, i.e., a seasonal clade 3C.3a1 H3N2 virus), and NYMC X-275 (derived from A/Michigan/45/2015, i.e., a clade 6B.1 pandemic 2009 H1N1 virus) were kindly provided by the National Institute for Biological Standards and Control (NIBSC; Potters Bar, UK). Other viruses in the study were generated by means of reverse genetics.
Construction of plasmids. The HA and NA vRNA segments were amplified by PCR with reverse transcription (RT-PCR) from each candidate vaccine virus and then inserted into the RNA polymerase I vector pHH21 (Neumann et al., 1999).
Construction of plasmid libraries. One to four random mutations were introduced into the six internal vRNA segments of the PR8-HY vaccine backbone by error-prone PCR using the GeneMorph Random Mutagenesis Kit (Agilent). The randomly mutated PCR products were then inserted into the pHH21 vector. The mutation rate of the generated plasmid libraries was confirmed by sequence analysis of individual bacterial colonies.
Virus library generation. All virus libraries were generated by using reverse genetics as described previously (Neumann et al., 1999). 100 μl of supernatant derived from transfected 293T cells were inoculated into embryonated chicken eggs to generate virus stocks; the titers of the virus stocks were determined by performing plaque assays in MDCK cells.
Serial passages of virus libraries in embryonated chicken eggs. Virus libraries were passaged in embryonated chicken eggs as shown in FIG. 10. Eggs were incubated for 48 h at 35° C. After each passage, the hemagglutination (HA) titer was determined; based on the titer, 1,000- or 10,000-fold dilutions of the virus libraries were used for the next passage in eggs.
Generation of virus stocks. 10-day-old embryonated chicken eggs were inoculated with 2×10³ PFU of each virus and the allantoic fluids were collected 48 h later. The HA and virus titers of the allantoic fluids were determined by means of HA and plaque assays, respectively.
Evaluation of viral growth kinetics. To analyze the growth kinetics of viruses in embryonated chicken eggs, 2×10² PFU of virus was inoculated into 10-day-old embryonated chicken eggs. The allantoic fluids of three eggs each were harvested at the indicated time points. HA and virus titers were determined by HA and plaque assays in MDCK cells, respectively.
Virus concentration and purification. Viruses (2×10² PFU) were grown in 10-day-old embryonated chicken eggs for 48 h. The allantoic fluids were harvested and clarified by centrifugation at 3,500 rpm. at 4° C. for 30 min. Then precipitate the virus by centrifugation at 18,500 rpm. for 2 h at 4° C. in a Beckman Type 19 rotor. The pellet was resuspended in 5 ml of PBS and loaded onto a 30 ml, 20-50% (20%, 30%, 35%, 40%, 45% and 50%) continuous sucrose gradient, which was centrifuged at 25,000 rpm. for 2 h at 4° C. in a Beckman SW32 rotor. The virus band was collected, diluted in PBS, and centrifugated at 25,000 rpm. for 2 h at 4° C. in a Beckman SW32 rotor to remove sucrose. The final virus pellet was resuspended in 300 μl PBS containing 0.1% β-propiolactone (BPL) at 4° C. overnight to inactivate the virus particles. Finally, the virus samples were incubated at 37° C. for 45 min to inactivate the BPL, and then aliquoted and stored at −80° C.
Determination of total protein yield. Total protein content of virus concentrate was determined by utilizing the Pierce BCA protein assay kit (Thermo Scientific) according to the manufacturer's instructions.
Deglycosylation of viral proteins. Viral protein was deglycosylated by use of PNGase F (New England Biolabs). Firstly, 2 μl of virus concentrate was denatured in a total volume of 10 μl according to the manufacture's instruction. Subsequently, the sample was incubated at 37° C. for 1 h with 2 μl of a 1/8 dilution of PNGase F in the GlycoBuffer provided by the manufacturer, and with NP-40 at a final concentration of 1%.
SDS-PAGE electrophoresis. Two microliter of virus concentrate was mixed with water to a total volume of 20 μl. Twenty microliter of loading dye containing 2% (v/v) β-mercaptoethanol as reducing agent was added to each protein sample. Samples were then heated to 95° C. for 5 min before loading onto the NuPage 4-12% Bris-Tris precast gel (Life technology). Electrophoresis was carried out with 1×MOPS buffer (Bio-Rad) at 150 V for 120 min and the gel was then stained with SYPRO-Ruby (Sigma). The protein yield was quantified by using Image J software (National Institutes of Health). The amount of total protein is the sum of the amounts of HA1, HA2, NP and M1; the HA amount is the sum of the amounts of HA1 and HA2. To calculate the HA content, we divided the HA amount by the amount of total protein and multiplied this value by the amount of total protein analyzed using gel electrophoresis.
Statistical analysis. For the analysis of the HA and virus titers at a single time-point, a one-way ANOVA followed by Tukey's Post Hoc test was used. To analyze the measurements of HA and growth titers at multiple-time points, for each mutant, the time-course data was analyzed using two-way ANOVA and defined contrasts to compare the different groups of viruses to each other at each time-point separately. In all cases, to stabilize the variance of the data, we transformed the HA titers to the log-2 scale, and virus titer measurements to the log-10 scale. The p-values were adjusted Holm's method, and considered significant if the p-values were less than 0.05. We used the R statistical package (www.r-project.org/), the Ismeans package (Length, 2016), and the Multcomp package (Hothorn, 2008). For the analysis of the viral total protein yield and HA content, statistical significance was determined.

### Results

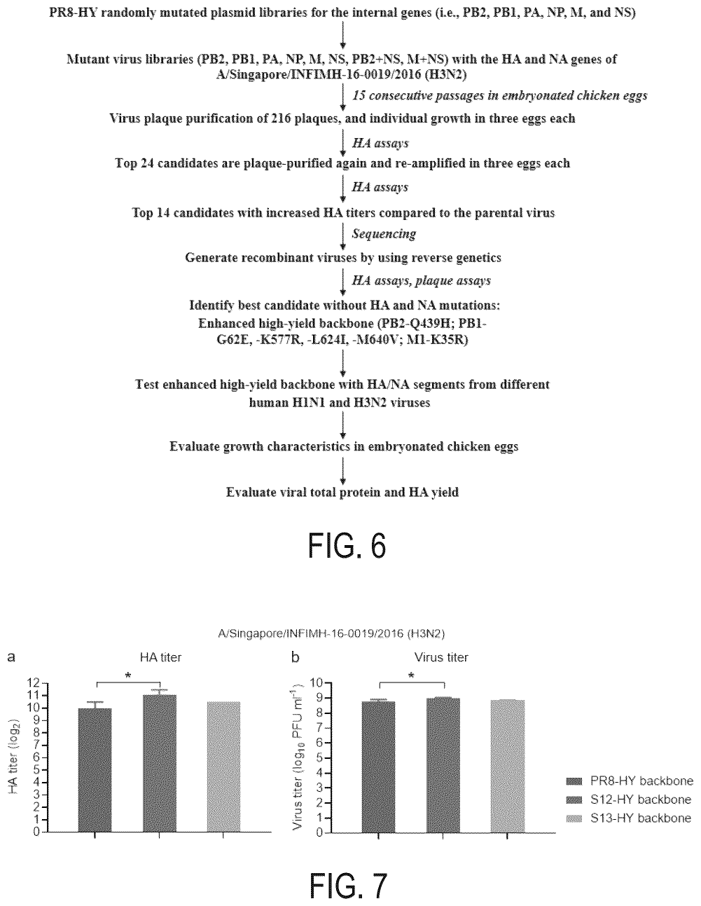

Isolation of enhanced high-yield vaccine virus backbones in embryonated chicken eggs. Previously, a high-yield influenza A vaccine virus backbone was prepared which possesses amino acid changes in several viral proteins (i.e., PB2-I504V, PB1-M40L/G180W, PA-R401K, NP-I116L, NS1-A30P/R118K) and nucleotide changes in the promoter regions of three viral RNAs (i.e., PB2-C4U, PB1-C4U, PA-C4U) (Ping et al., 2015). Starting with the PR8-HY vaccine virus backbone previously generated, a similar strategy was employed to further improve the PR8-HY vaccine virus backbone for efficient replication in embryonated chicken eggs. First, error-prone PCR was performed to introduce one-to-four random amino acid changes, on average, into the viral proteins encoded by the internal viral RNA segments (Ping et al., 2015). By using established reverse genetics approaches (Neumann et al., 1999), the mutated cDNAs libraries were used to generate the following eight virus libraries (FIG. 6, FIG. 10): six individual virus libraries possessing random mutations in each of the internal vRNAs (that is, PB2, PB1, PA, NP, M and NS); one virus library possessing random mutations in the PB2 and NS vRNAs because the PB2 and NS1 proteins (encoded by the NS vRNA) may affect viral growth properties (Arai et al., 2019; Ma et al., 2010); and one library possessing random mutations in the M and NS vRNAs because the M1 protein (encoded by the M vRNA) has been associated with high-yield properties (Klimov et al., 1991; Ramminuninair et al. 2013). All eight virus libraries possessed the HA and NA vRNAs of a recent human H3N2 vaccine strain, i.e., A/Singapore/INFIMH-16-0019/2016 (H3N2; Singapore).

To select variants with enhanced high-yield properties, the eight individual virus libraries were passaged 15 times in embryonated chicken eggs (FIG. 6, FIG. 10). In parallel, aliquots of the eight individual virus libraries after the second passage were combined to generate a ‘mixed library’, which was then subjected to 15 additional passages in embryonated chicken eggs (FIG. 10). After the last passage, we performed plaque assays in MDCK cells and randomly selected more than 20 virus plaques from each library, resulting in a total of 216 plaque-purified viruses. These viruses were each amplified in three embryonated chicken eggs, followed by hemagglutination (HA) assays. Twenty-four mutants exhibited HA titers that were at least two-fold higher than those of the parental PR8-HY virus (i.e., PR8-HY possessing the HA and NA vRNAs of Singapore). These enhanced high-yield candidates were again plaque-purified and amplified in three embryonated chicken eggs each. We collected samples at 36 h and 48 h post-infection to assess the HA titers and identified 14 viruses whose HA titers were at least two-fold higher compared to virus based on the parental PR8-HY backbone (Table 1).

Next, the entire viral genomes of the top 14 enhanced high-yield candidates were sequenced most of which originated from the PB1 virus library (Table 1). Interestingly, most of the enhanced high-yield candidates from the PB1 virus library possessed PB2-Q439H and/or M1-K35R mutations, suggesting that these mutations emerged in the PB1 virus library. In addition, several of the enhanced high-yield candidates from the PB1 virus library also encoded PB1-G62E/K577R/L624I/M640V mutations. Several other mutations in the PB2, PB1, PA, M1, M2, and NS1 proteins were detected in subsets of the top 14 enhanced high-yield candidates. Although we did not intentionally introduce mutations into the HA and NA genes, several mutations were detected in the HA and NA proteins (Table 1). In viruses derived from the PB1 virus library, the HA-K121E/T2031 amino acid changes were detected frequently (amino acid numbers refer to mature H3 HA). For NA, the NA-I212T and V303I mutations were detected frequently.

TABLE 1
Amino acid changes in the top 14 increased high-yield candidates

| | | | Influenza virus protein | | | | | | | |
|---|---|---|---|---|---|---|---|---|---|---|
| Virus | Virus | HA titer | | | | | | | HA (H3 | |
| isolate | library | (2) | PB2 | PB1 | PA | M1 | M2 | NS1 | numbering^{a}) | NA |
| PR8-HY | — | 9.0~9.5 | n/a | n/a | n/a | n/a | n/a | n/a | n/a | n/a |
| backbone | | | | | | | | | | |
| #34 | Mixed | 9.5~11.5 | I105M | | | | | | I140M | V303I |
| #12 | PB2 + | 10.5~11.5 | V504I | | | | | | S124N | V303I |
| | NS | | | | | | | | | |
| #3 | M | 9.5~11.5 | | K479M | | | | | I140M | D221N |
| #2 | PB1 | 10.0~11.5 | Q439H | | | K35R | R12K/ | | K121E/ | V303I |
| | | | | | | | R54P | | T203I | |
| #3 | PB1 | 9.5~10.5 | Q439H | L396I | | K35R | | | K121E/ | I212T |
| | | | | | | | | | T203I | |
| #5 | PB1 | 9.5~11.5 | Q439H | G62E/ | D294N | K35R | | | K121E/ | I212T |
| | | | | K577R/ | | | | | T203I | |
| | | | | L624I/ | | | | | | |
| | | | | M640V | | | | | | |
| #8 | PB1 | 10.5~11.0 | Q439H | | | K35R | | | K121E/ | I212T |
| | | | | | | | | | T203I | |
| #9 | PB1 | 10.5~11.5 | Q439H | G62E/ | | | | | K121E/ | V303I |
| | | | | K577R/ | | | | | T203I | |
| | | | | L624I/ | | | | | | |
| | | | | M640V | | | | | | |
| #10 | PB1 | 10.5~11.0 | Q439H/ | M655I | | K35R | | | K121E/ | V303I |
| | | | A624S | | | | | | T203I | |
| #11 | PB1 | 9.5~11.5 | Q439H | G62E/ | | K3SR | | | I140M/ | V303I |
| | | | | K577R/ | | | | | V309I | |
| | | | | L624I/ | | | | | | |
| | | | | M640V | | | | | | |
| #12 | PB1 | 8.5~10.5 | Q439H | | | K35R | | E75G/ | I140M | |
| | | | | | | | | K78R | | |
| #14 | PB1 | 10.5~11.5 | Q439H | L396I/ | | | | | K121E/ | I212T |
| | | | | K479M | | | | | T203I | |
| #16 | PB1 | 10.5~11.5 | | G62E/ | | | R12K | | K121E/ | V303I |
| | | | | T528N/ | | | | | T203I | |
| | | | | K577R/ | | | | | | |
| | | | | L624I/ | | | | | | |
| | | | | M640V | | | | | | |
| #17 | PB1 | 10.5~11.0 | | G62E/ | R124K/ | K35R | G16E | | K121E/ | I212T |
| | | | | K577R/ | K339R | | | | T203I | |
| | | | | L624I/ | | | | | | |
| | | | | M640V | | | | | | |

^{a}Numbers refer to the mature HA protein.

TABLE 2
HA titers of high-yield candidates in embryonated
chicken eggs at 36 h and 48 h post-infection

| | | HA titers at the indicated time point (2ⁿ) | | | | | |
|---|---|---|---|---|---|---|---|
| | Candidate | 36 h post-infection | | | 48 h post-infection | | |
| Virus library | no.¹ | Egg 1 | Egg 2 | Egg 3 | Egg 1 | Egg 2 | Egg 3 |
| PR8-HY | | 9.0 | 9.0 | 9.5 | 9.0 | 9.5 | 8.5 |
| (Parental | | | | | | | |
| virus) | | | | | | | |
| Mixed | 14 | 9.5 | 10.5 | 10.0 | 10.0 | 10.0 | 10.5 |
| | 34 | 11.5 | 10.5 | 9.5 | 11.5 | 10.0 | 10.5 |
| PB2 + NS | 3 | 11.5 | 10.0 | 10.0 | 9.5 | 10.0 | 10.0 |
| | 11 | 9.5 | 9.5 | 10.5 | 10.0 | 11.0 | 9.5 |
| | 12 | 10.5 | 10.5 | 10.5 | 10.5 | 11.5 | 11.0 |
| M | 3 | 11.5 | 9.5 | 11.0 | 10.5 | 10.5 | 10.5 |
| NP | 2 | 10.0 | 9.5 | 10.0 | 10.5 | 10.5 | 9.5 |
| | 8 | 10.0 | 9.5 | 10.0 | 9.5 | 9.5 | 10.0 |
| PA | 20 | 9.5 | 10.5 | 9.5 | 10.0 | 10.0 | 10.0 |
| PB1 | 2 | 11.0 | 11.0 | 10.5 | 10.0 | 10.0 | 11.5 |
| | 3 | 10.5 | 10.5 | 9.5 | 10.5 | 10.5 | 10.5 |
| | 5 | 9.5 | 10.0 | 10.5 | 10.5 | 11.5 | 10.5 |
| | 7 | 11.0 | 9.5 | 10.5 | 10.5 | 9.5 | 10.5 |
| | 8 | 10.5 | 11.0 | 11.0 | 10.0 | 10.0 | 11.0 |
| | 9 | 10.5 | 11.5 | 11.5 | 11.5 | 10.5 | 10.5 |
| | 10 | 10.5 | 10.5 | 11.0 | 10.5 | 10.5 | 10.5 |
| | 11 | 9.5 | 10.5 | 10.5 | 11.5 | 11.5 | 10.5 |
| | 12 | 10.5 | 8.5 | 10.5 | 10.5 | 10.5 | 10.5 |
| | 13 | 10.5 | 12.0 | 9.5 | 10.0 | 10.0 | 10.0 |
| | 14 | 11.5 | 10.5 | 10.5 | 10.5 | 10.5 | 10.5 |
| | 15 | 9.5 | 10.5 | 10.5 | 10.5 | 9.5 | 11.0 |
| | 16 | 10.5 | 11.5 | 11.0 | 11.0 | 10.5 | 10.5 |
| | 17 | 11.0 | 11.0 | 11.0 | 10.5 | 10.5 | 10.5 |
| | 18 | 9.5 | 10.5 | 10.5 | 9.5 | 8.0 | 10.5 |

¹The top 14 candidates are marked in bold-type.

Identification of mutation(s) that increase virus replication in embryonated chicken eggs. The top 14 enhanced high-yield candidates encode various combinations of amino acid changes in the internal viral proteins. To assess their relative contributions to increased virus replication, we generated recombinant viruses with different amino acid changes (RG-S3-RG-S16; Table 3). The parental HY-PR8 virus encoding the HA and NA proteins of Singapore was generated and tested twice (RG-S1, RG-S2). To assess virus replication, three embryonated chicken eggs were each inoculated with an aliquot of the supernatant derived from plasmid-transfected 293T cells and HA titers were measured 48 hours later (Table 3). For one of the egg-amplified samples (that is, the sample with the highest HA titer among the three replicates), plaque assays were performed in MDCK cells (Table 3). Compared to the parental PR8-HY virus backbone (RG-S1, RG-S2), combinations of mutations in the internal proteins could increase virus titers up to 4.3-fold (Table 3).

TABLE 3
Enhanced high-yield vaccine candidates of A/Singapore/INFIMH-16-0019/2016
(H3N2) with combinations of mutations identified in virus library screens

| | Amino acid changes compared to PR8-HY virus with the HA and NA | | | | | | | | | | |
|---|---|---|---|---|---|---|---|---|---|---|---|
| Recombinant | genes of A/Singapore/INFIMH-16-0019/2016 | | | | | | HA titer | Viral titer | | | |
| virus | PB2 | PB1 | PA | NP | M | NS | (2ⁿ)^{b} | (PFU/ml)^{c} | Fold-increased | | |
| RG-S1a | — | — | — | — | — | — | 10.5* | 10.5 | 10.0 | 4.0 × 10\hat{ }8 | |
| RG-S2a | — | — | — | — | — | — | 10.0 | 10.0 | 10.5* | 4.2 × 10\hat{ }8 | |
| RG-S3 | I105M | — | — | — | — | — | 9.5 | 10.5* | 9.5 | 7.0 × 10\hat{ }8 | 1.7 |
| RG-S4 | V504I | — | — | — | — | — | 9.5 | 9.0 | 10.0 | 5.0 × 10\hat{ }8 | 1.2 |
| RG-S5 | — | K479 M | — | — | — | — | 9.5 | 9.5 | 10.0* | 3.0 × 10\hat{ }8 | <1.0 |
| RG-S6 | Q439H | — | — | — | K35R(M1)/ | — | 9.5 | 10.5 | 10.5* | 8.0 × 10\hat{ }8 | 1.9 |
| | | | | | R12K/R54P(M2) | | | | | | |
| RG-S7 | Q439H | L396I | — | — | K35R(M1) | — | 9.5 | 11.0* | 10.5 | 8.0 × 10\hat{ }8 | 1.9 |
| RG-S8 | Q439H | G62E/K577R/ | D294N | — | K35R(M1) | — | 10.5* | 10.5 | 0 | 6.0 × 10\hat{ }8 | 1.4 |
| | | L624I/M640V | | | | | | | | | |
| RG-S9 | Q439H | — | — | — | K35R(M1) | — | 11.0* | 10.5 | 9.0 | 1.5 × 10\hat{ }9 | 3.6 |
| RG-S10 | Q439H | G62E/K577R/ | — | — | — | — | 9.5 | 10.0 | 10.5* | 9.0 × 10\hat{ }8 | 2.1 |
| | | L624I/M640V | | | | | | | | | |
| RG-S11 | Q439H/ | M655I | — | — | K35R(M1) | — | 9.5 | 10.5* | 9.5 | 8.1 × 10\hat{ }8 | 1.9 |
| | A624S | | | | | | | | | | |
| RG-S12 | Q439H | G62E/K577R/ | — | — | K35R(M1) | — | 11.0* | 10.5 | 10.5 | 1.8 × 10\hat{ }9 | 4.3 |
| | | L624I/M640V | | | | | | | | | |
| RG-S13 | Q439H | — | — | — | K35R(M1) | E75G/ | | | | | |
| | | | | | | K78R | | | | | |
| (NS1) | 10.5 | 11.0 | 11.5* | 1.6 × 10\hat{ }9 | 3.9 | | | | | | |
| RG-S14 | Q439H | L396I/K479M | — | — | — | — | 9.5 | 9.5 | 10.5* | 3.0 × 10\hat{ }8 | <1.0 |
| RG-S15 | — | G62E/T528N/ | — | — | R12K(M2) | — | 10.0* | 9.5 | 9.5 | 3.0 × 10\hat{ }8 | <1.0 |
| | | K577R/L624I/ | | | | | | | | | |
| | | M640V | | | | | | | | | |
| RG-S16 | — | G62E/K577R/ | R124K/ | — | K35R(M1)/ | — | 9.5 | 9.5* | 9.5 | 5.0 × 10\hat{ }8 | 1.2 |
| | | L624I/M640V | K339R | | G16E(M2) | | | | | | |

^{a}The control virus (possessing the internal genes of PR8-HY and the HA and NA genes of A/Singapore/INFIMH-16-0019/2016) was generated and titrated twice.
^{b}The HA titers were determined by performing HA assays with 0.5% turkey red blood cells.
^{c}The viral titers in MDCK cells were determined for the allantoic fluids indicated by an asterisk.
^{d}Fold-increase was the calculated by the viral titer divided by the mean of the control viruses.

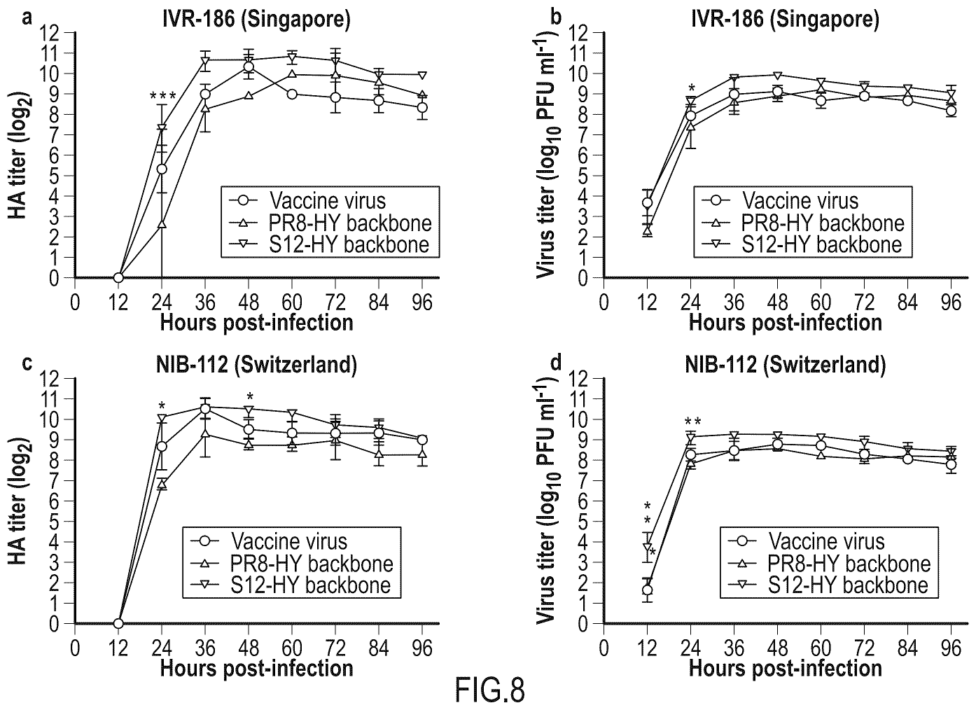

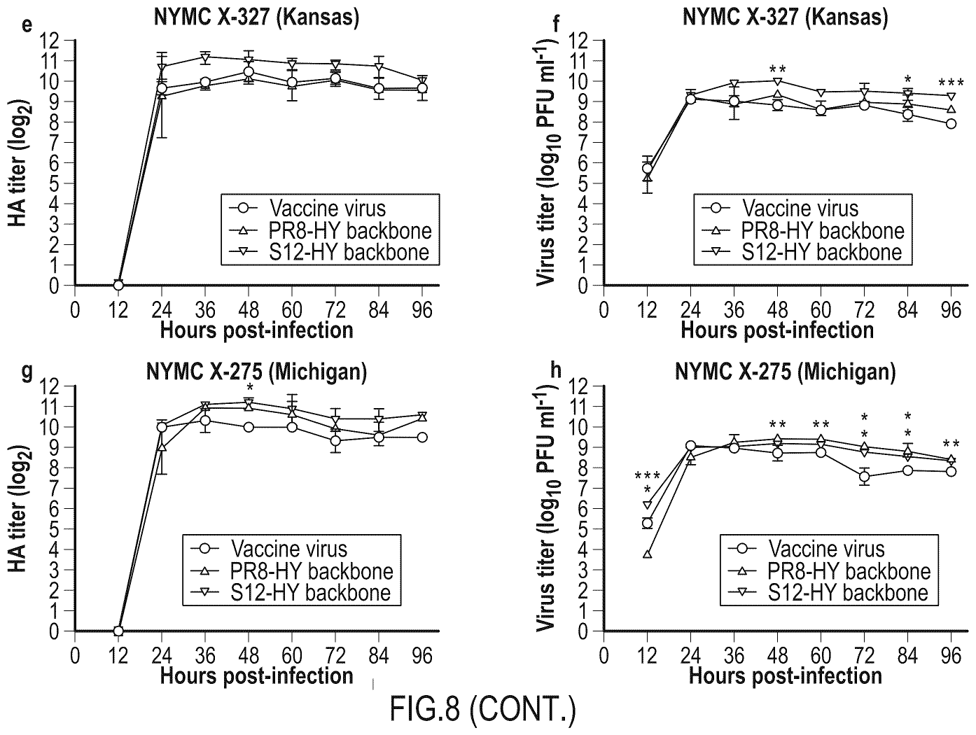

Selection of high-yield vaccine virus backbones for additional studies. Based on the data shown in Table 3, we focused on the RG-S12 and RG-S13 enhanced high-yield candidates which showed the greatest increases in virus titers among the candidates with mutations in the internal proteins (Table 3). After amplification in embryonated chicken eggs, the resulting virus stocks (named S12- and S13-HY, respectively) were then tested for their HA and virus titers in embryonated chicken eggs (FIG. 7). For S12-HY (but not for S13-HY), the HA and virus titers were significantly higher than for PR8-HY (0-0.05).
Evaluation of candidate vaccine viruses in embryonated chicken eggs. The enhanced high-yield vaccine backbone S12-HY was developed with the HA and NA vRNAs of Singapore virus. To determine if this enhanced high-yield vaccine virus backbone also increased the titers of other vaccine viruses, the S12-HY vaccine backbone was combined with the HA and NA vRNAs of three other human influenza vaccine strains, A/Switzerland/8060/2017 (H3N2; Switzerland; clade 3C.2a2), A/Kansas/14/2017 (H3N2; Kansas; clade 3C.3a1) and A/Michigan/45/2015 (H1N1; Michigan; clade 6B.1) (Table 4). As controls, the HA and NA vRNAs of Switzerland, Kansas and Michigan viruses were tested in the parental PR8-HY backbone (Table 6). Moreover, egg-grown IVR-186 (Singapore, H3N2), NIB-112 (Switzerland, H3N2), NYMC X-327 (Kansas, H3N2) and Michigan (NYMC X-275, H1N1) vaccine viruses were obtained from the National Institute for Biological Standards and Control (NIBSC; Potters Bar, UK) (Table 4). First, virus stocks were generated by pooling the allantoic fluids of three inoculated embryonated chicken eggs. The titers of the virus stocks were determined by using plaque assays in MDCK cells. Then, ten-day-old embryonated chicken eggs were inoculated with 2×10² plaque-forming units (PFU) of the respective viruses and incubated at 35° C. for the indicated periods of time (FIG. 8). Viruses with the enhanced high-yield (S12-HY) vaccine backbone replicated more efficiently than IVR-186 (Singapore), NIB-112 (Switzerland), NYMC X-327 (Kansas), and NYMC X-275 (Michigan) at one or more time points (FIG. 8, *p<0.05, **p<0.01, ***p<0.001). Moreover, the enhanced S12-HY vaccine backbone conferred higher HA and/or virus titers than the parental PR8-HY backbone, although not all differences were statistically significant (FIG. 8) (*p<0.05, **p<0.01, ***p<0.001).

TABLE 4
Summary of viruses tested in this study

| | Virus | | | | | | | | | | Passage |
|---|---|---|---|---|---|---|---|---|---|---|---|
| Original strain | description | Subtype | HA | NA | PB2 | PB1 | PA | NP | M | NS | history |
| A/Singapore/INFIMH- | Egg-grown | H3N2 | A/Singapore/ | A/Singapore/ | PR8 | PR8 | PR8 | PR8 | PR8 | PR8 | E8a |
| 16-0019/2016 (IVR- | vaccine virus | | INFIMH-16- | INFIMH-16- | | | | | | | |
| 186) | | | 0019/2016 | 0019/2016 | | | | | | | |
| | Recombinant | H3N2 | A/Singapore/ | A/Singapore/ | PR8- | PR8- | PR8- | PR8- | PR8- | PR8- | E1 |
| | virus | | INFIMH-16- | INFIMH-16- | HY* | HY | HY | HY | HY | HY | |
| | | | 0019/2016 | 0019/2016 | | | | | | | |
| | Recombinant | H3N2 | A/Singapore/ | A/Singapore/ | S12- | S12- | S12- | S12- | S12- | S12- | E1 |
| | virus | | INFIMH-16- | INFIMH-16- | HY* | HY | HY | HY | HY | HY | |
| | | | 0019/2016 | 0019/2016 | | | | | | | |
| A/Switzerland/8060/2017 | Egg-grown | H3N2 | A/Switzerland/ | A/Switzerland/ | PR8 | PR8 | PR8 | PR8 | PR8 | PR8 | E6a |
| (NIB-112) | vaccine virus | | 8060/2017 | 8060/2017 | | | | | | | |
| | Recombinant | H3N2 | A/Switzerland/ | A/Switzerland/ | PR8- | PR8- | PR8- | PR8- | PR8- | PR8- | E1 |
| | virus | | 8060/2017 | 8060/2017 | HY | HY | HY | HY | HY | HY | |
| | Recombinant | H3N2 | A/Switzerland/ | A/Switzerland/ | S12- | S12- | S12- | S12- | S12- | S12- | E1 |
| | virus | | 8060/2017 | 8060/2017 | HY | HY | HY | HY | HY | HY | |
| A/Kansas/14/2017 | Egg-grown | H3N2 | A/Kansas/14/ | A/Kansas/14/ | PR8 | PR8 | PR8 | PR8 | PR8 | PR8 | E17a |
| (NYMC X-327) | vaccine virus | | 2017 | 2017 | | | | | | | |
| | Recombinant | H3N2 | A/Kansas/14/ | A/Kansas/14/ | PR8- | PR8- | PR8 | PR8- | PR8- | PR8- | E1 |
| | virus | | 2017 | 2017 | HY | HY | HY | HY | HY | HY | |
| | Recombinant | H3N2 | A/Kansas/14/ | A/Kansas/14/ | S12- | S12- | S12- | S12- | S12- | S12- | E1 |
| | virus | | 2017 | 2017 | HY | HY | HY | HY | HY | HY | |
| A/Michigan/45/2015 | Egg-grown | pdmH1N1 | A/Michigan/ | A/Michigan/ | PR8 | A/Michigan | PR8 | PR8 | PR8 | PR8 | E11a |
| (NYMC X-275) | vaccine virus | | 45/2015 | 45/2015 | | 45/2015 | | | | | |
| | Recombinant | pdmH1N1 | A/Michigan/ | A/Michigan/ | PR8- | PR8- | PR8- | PR8- | PR8 | PR8- | E1 |
| | virus | | 45/2015 | 45/2015 | HY | HY | HY | HY | -HY | HY | |
| | Recombinant | pdmH1N1 | A/Michigan/ | A/Michigan/ | S12- | S12- | S12- | S12- | S12 | S12- | E1 |
| | virus | | 45/2015 | 45/2015 | HY | HY | HY | HY | -HY | HY | |

*High-yield backbones tested in this study.
aPassage history of the vaccine viruses obtained from NIBSC.

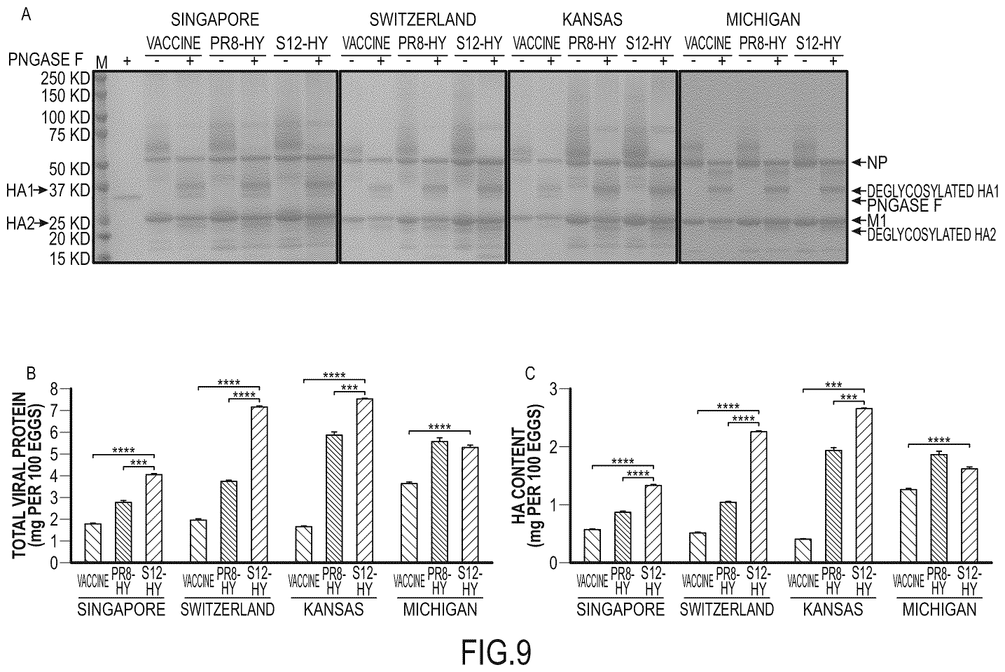

Evaluation of total viral protein yield and HA content. Total viral protein yield, and in particular the amount of HA, are important parameters for vaccine production. Thus, the total viral protein yield and HA content of the high-yield vaccine candidates propagated in embryonated chicken eggs were compared. The embryonated chicken eggs were inoculated with 2×10² PFU viruses and allantoic fluids were then concentrated and purified by using sucrose gradient centrifugation. Total viral protein yield was determined by use of the Pierce BCA Protein Assay Kit (Thermo Scientific, Rochester, NY, USA). The purified virus samples were then treated with PNGase F to deglycosylate HA, so that the molecular weight of HA2 can be distinguished from that of M1. Samples were then analyzed through SDS-polyacrylamide gel electrophoresis (SDS-PAGE) (FIG. 9a). The amounts of HA1, HA2, NP and M1 were determined by using Image J software (National Institutes of Health. The S12-HY backbone yielded significantly higher amounts of total viral protein (FIG. 9b) and HA content (FIG. 9c) when compared with the three H3N2 vaccine viruses, i.e., IVR-186 (Singapore), NIB-112 (Switzerland), and NYMC X-327 (Kansas), and when compared with the control viruses based on PR8-HY backbone (***p<0.001, ****p<0.0001). For the H1N1 vaccine virus, i.e., NYMC X-275 (Michigan), the S12-HY backbone yield significantly higher amounts of total virus protein (FIG. 9b) and HA content (FIG. 9c) when compared with the vaccine virus, but not when compared with the virus based on PR8-HY backbone. Collectively, these data demonstrated that the S12-HY vaccine virus backbone could increase the HA content of seasonal influenza vaccine, specifically those of the H3N2 subtype.

### DISCUSSION

The enhanced high-yield vaccine virus backbone, S12-HY, significantly increased the HA content and viral titers of several seasonal influenza A vaccine viruses in embryonated chicken eggs compared to authentic vaccine viruses obtained from NIBSC. Moreover, the enhanced high-yield vaccine virus backbone improved the HA content and viral growth properties compared to the parental high-yield PR8 backbone in some comparisons, but not in all of them. Collectively, the enhanced high-yield vaccine virus backbone may be used in the future to increase the yield of seasonal influenza vaccines.

The enhanced high-yield vaccine virus backbone possesses six amino acid changes in three different viral proteins compared with the parental PR8-HY vaccine backbone (i.e., PB2-Q439H, PB1-G62E/K577R/L624I/M640V, and M1-K35R). The PB2-Q439 residue is highly conserved among human, swine, and avian influenza A virus proteins; the histidine residue at this position of the enhanced S12-HY vaccine virus backbone has not been detected among circulating influenza viruses (Table 5). Similarly, the PB1-G62 and PB1-K577 residues are also highly conserved among human, swine, and avian influenza A virus proteins (Table 5). The PB1-L624 residue is highly conserved among human and swine influenza A viruses, while ˜24% of avian influenza viruses encode an arginine at this position. Residue PB1-M640 (encoded by the parental PR8-HY vaccine backbone) is rarely found among human, swine, and avian influenza A viruses; interestingly, the valine residue selected from randomly mutated PB1 protein is encoded by the majority of human, swine, and avian influenza A viruses (Table 5). The M1-K35 residue is extremely common in nature, and the arginine residue detected in the S12-HY backbone has been detected in only a small number of circulating viruses (Table 5).

TABLE 5
Frequency in human, swine, and avian influenza A viruses of
amino acid changes in the S12-HY vaccine virus backbone

| | | | Human H1N1 | | Human H3N2 | | Swine influenza | | Avian influenza | |
|---|---|---|---|---|---|---|---|---|---|---|
| | | | influenza viruses | | influenza viruses | | viruses | | viruses | |
| | Position | | Number | | Number | | Number | | Number | |
| Viral | and | Amino | of | Frequency | of | Frequency | of | Frequency | of | Frequency |
| protein | mutation | acid | viruses | (%) | viruses | (%) | viruses | (%) | viruses | (%) |
| PB2 | Q439H | Q | 14187 | 100.00% | 20631 | 100.00% | 7744 | 100.00% | 19188 | 100.00% |
| | | H | 0 | 0 | 0 | 0 | 0 | 0 | 0 | 0 |
| PB1 | G62E | G | 13767 | 99.93% | 20510 | 99.70% | 7579 | 99.65% | 17792 | 98.65% |
| | | E | 1 | <0.01% | 40 | 0.19% | 0 | 0 | 68 | 0.38% |
| | | R | 1 | <0.01% | 3 | 0.01% | 8 | 0.11% | 60 | 0.33% |
| | | D | 4 | 0.02% | 0 | 0 | 0 | 0 | 0 | 0 |
| | | K | 0 | 0 | 0 | 0 | 8 | 0.11% | 114 | 0.63% |
| | | Others | 1 | <0.01% | 4 | 0.02% | 2 | 0.03% | 0 | 0 |
| | | (S/W) | | | | | | | | |
| | K577R | K | 13735 | 99.70% | 20514 | 99.76% | 7484 | 98.40% | 13452 | 74.59% |
| | | R | 20 | 0.14% | 26 | 0.12% | 90 | 1.18% | 215 | 1.19% |
| | | N | 8 | 0.06% | 5 | 0.02% | 22 | 0.29% | 11 | 0.06% |
| | | Q | 1 | <0.01% | 8 | 0.03% | 2 | 0.03% | 0 | 0 |
| | | E | 7 | 0.05% | 2 | <0.01% | 2 | 0.03% | 17 | 0.09% |
| | | M | 1 | <0.01% | 0 | 0 | 1 | 0.01% | 21 | 0.12% |
| | | I | 0 | 0 | 0 | 0 | 0 | 0 | 46 | 0.26% |
| | | L | 0 | 0 | 0 | 0 | 0 | 0 | 4260 | 23.62% |
| | | Others | 4 | 0.03% | 4 | 0.02% | 3 | 0.04% | 0 | 0 |
| | | (S/T) | | | | | | | | |
| | L624I | L | 8096 | 58.76% | 20555 | 99.96% | 7595 | 99.86% | 13678 | 75.84% |
| | | I | 1 | <0.01% | 5 | 0.02% | 9 | 0.04% | 7 | 0.04% |
| | | C | 5676 | 41.19% | 0 | 0 | 0 | 0 | 1 | <0.01% |
| | | R | 0 | 0 | 0 | 0 | 0 | 0 | 4325 | 23.98% |
| | | K | 0 | 0 | 0 | 0 | 0 | 0 | 2 | 0.01% |
| | | F | 0 | 0 | 0 | 0 | 0 | 0 | 6 | 0.03% |
| | | V | 0 | 0 | 0 | 0 | 0 | 0 | 3 | 0.02% |
| | | Others | 1 | <0.01% | 0 | 0 | 0 | 0 | 0 | 0 |
| | | (Y/Q) | | | | | | | | |
| | M640V | M | 15 | 0.11% | 2 | <0.01% | 2 | 0.03% | 12 | 0.07% |
| | | V | 8040 | 58.36% | 20501 | 99.70% | 7390 | 97.16% | 13622 | 75.53% |
| | | I | 41 | 0.30% | 56 | 0.27% | 202 | 2.66% | 35 | 0.19% |
| | | L | 1 | <0.01% | 3 | 0.01% | 7 | 0.09% | 13 | 0.07% |
| | | S | 1 | <0.01% | 0 | 0 | 0 | 0 | 4327 | 23.99% |
| | | N | 5678 | 41.21% | 0 | 0 | 0 | 0 | 0 | 0 |
| | | A | 0 | 0 | 0 | 0 | 3 | 0.04% | 15 | 0.08% |
| | | Others | 0 | 0 | 0 | 0 | 0 | 0 | 4 | 0.02% |
| | | (D/F) | | | | | | | | |
| M1 | K35R | K | 12637 | 99.99% | 23837 | 99.98% | 988 | 98.90% | 20239 | 99.91% |
| | | R | 1 | <0.01% | 2 | <0.01% | 10 | 1.00% | 17 | 0.08% |
| | | N | 0 | 0 | 0 | 0 | 1 | 0.10% | 0 | 0 |

Amino acid 439 of PB2 is located in the cap-binding domain of the PB2 protein, while residues 62, and 577/624/640 are located in the fingers and thumb domain of the PB1 protein, respectively (Fan et al., 2019). A previous study demonstrated that PB1-577E enhances the viral polymerase activity in human cells (Kamiki et al., 2018) but whether the PB2 and PB1 mutations identified in the study can affect virus replication and polymerase activity remains to be studied further. The M1 protein is an abundant structural component of the influenza A viral particle and plays a role in the regulation of vRNP nucleocytoplasmic shuttling (Martin et al., 1991). Most viruses isolated in the study possessed the M1-K35R mutation after several passages in embryonated chicken eggs, suggesting selection of this mutation. The K35R mutation in M1 protein increased the ubiquitination of M1 (Mahesutihan et al., 2018), but the role of this mutation in viral replication remains unknown.

### REFERENCES

Arai et al., PLoS Pathog., 15:e1007919 (2019).
Avery's Drug Treatment: Principles and Practice of Clinical Pharmacology and Therapeutics, 3rd edition, ADIS Press, Ltd., Williams and Wilkins, Baltimore, MD (1987).
Aymard-Henry et al., Virology: A Practical Approach, Oxford IRL Press, Oxford, 119-150 (1985).
Bachmeyer, Intervirology, 5:260 (1975).
Berkow et al., eds., The Merck Manual, 16th edition, Merck & Co., Rahway, NJ (1992).
Fan et al., Nature, 573:287 (2018).
Hatta et al., Science, 293:1840 (2001).
Health OW. Fact sheet: influenza (seasonal). 2018. http:wwwwhoint/Accessed 9 Jul. 2019 www.who.int/en/news-room/fact-sheets/detail/influenza-(seasonal).
Horimoto et al., J. Virol., 68:3120 (1994).
Horimoto et al., Vaccine, 24:3669 (2006).
Hothorn et al., Biom. J., 50:346 (2008).
Iuliano et al., Lancet, 391:1285 (2018).
Kamiki et al., Viruses, 10 (2018).
Keitel et al., in Textbook of Influenza, eds. Nickolson, K. G., Webster, R. G., and Hay, A. (Blackwell, Oxford), pp. 373-390 (1998).
Klimov et al., Virus Res., 19:105 (1991).
Laver & Webster, Virology, 69:511 (1976).
Length, Journal of statistical software, 69:1 (2016).
Ma et al. J. Virol., 84:2122 (2010).
Mahesutihan et al., Virol. Sin, 33:440 (2018).
Martin et al., Cell., 67:117 (1991).
Neumann et al., Adv. Virus Res., 5.3:265 (1999).
Neumann et al., J. Gen. Virol., 83:2635 (2002).
Neumann et al., J. Virol., 71:9690 (1997).
Neumann et al., Virology, 287:243 (2001).
Neumann et al., Proc Natl Acad Sci USA, 96:9345 (1999).
Osol (ed.), Remington's Pharmaceutical Sciences, Mack Publishing Co., Easton, PA 1324-1341 (1980).
Ping et al., Proc Natl Acad Sci USA, 113:E8296 (2016).
Ping et al., Nature Communications, 6:8148 (2015).
Ramanunninair et al., PLoS One, 8:e65955 (2013).
Sugawara et al., Biologicals, 30:303 (2002).
Webby & Webster et al., Science, 302:1519 (2003).
Wood & Robertson, Nat. Rev. Microbiol., 2:842 (2004).
World Health Organization TSR No. 673 (1982).
World Health Organization. Confirmed human cases of avian influenza A (H5N1). http://www.who.int/csr/disease/avian_influenza/country/en/index.html

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. An isolated recombinant influenza virus having PA, PB1, PB2, NP, NS, and M viral segments from a first influenza vaccine virus isolate, a heterologous, recombinant or chimeric influenza virus NA viral segment, and a heterologous, recombinant or chimeric HA viral segment, wherein the PB1 viral segment encodes a PB1 polypeptide having a residue other than glycine (G) or alanine (A) at position 62, a residue other than lysine (K) at position 577, a residue other than leucine (L) or valine (V) at position 624, and/or a residue other than methionine (M) at position 640, wherein the PB2 segment encodes a PB2 polypeptide having a residue other than glutamine (Q) at position 439, or wherein the M segment encodes a M1 polypeptide having a residue other than K at position 35; or a combination thereof.

2. The isolated recombinant influenza virus of claim 1 wherein the residue at position 62 in PB1 comprises glutamic acid (E), aspartic acid (D), K, arginine (R), or histidine (H).

3. The isolated recombinant influenza virus of claim 1, wherein the residue at position 577 in PB1 comprises R, K, D, E or H.

4. The isolated recombinant influenza virus of claim 1, wherein the residue at position 624 in PB1 comprises isoleucine (I), alanine (A), G, or threonine (T).

5. The isolated recombinant influenza virus of claim 1, wherein the residue at position 640 in PB1 comprises V, I, A, G, or T.

6. The isolated recombinant influenza virus of claim 1, wherein the residue at position 439 in PB2 comprises H, R, K, D, or E.

7. The isolated recombinant influenza virus of claim 1, wherein the residue at position 35 in M1 comprises R, H, D or E.

8. The isolated recombinant influenza virus of claim, 1 wherein the PB2 viral segment further encodes a PB2 with a V at position 504.

9. The isolated recombinant influenza virus of claim 1, wherein the PA viral segment encodes a PA with a K at position 401.

10. The isolated recombinant influenza virus of claim 1, wherein the PB1 viral segment further encodes a PB1 having a leucine at position 40 and/or tryptophan (W) at position 180.

11. The isolated recombinant influenza virus of claim 1, wherein the NP viral segment encodes a NP polypeptide having leucine at position 116.

12. The isolated recombinant influenza virus of claim 1, wherein the NS viral segment encodes a NS1 polypeptide having a proline at position 30 and/or a lysine at position 118.

13. The isolated recombinant influenza virus of claim 1, wherein at least one of the PA, PB1, PB2, NP, NS, and M viral segments has a C to U promoter mutation.

14. The isolated recombinant influenza virus of claim 13, wherein the PB1, PB2 and/or PA viral segment(s) comprise a C4U promoter mutation.

15. The isolated recombinant influenza virus of claim 1, wherein the NA viral segment and the HA viral segment are from the same influenza virus isolate.

16. The isolated recombinant influenza virus of claim 1, wherein at least one of the PA, PB1, PB2, NP, NS, and M viral segments comprise: a PB1 with at least 95% amino acid sequence identity to the PB1 encoded by SEQ ID NO:2; a PB2 with at least 95% amino acid sequence identity to the PB2 encoded by SEQ ID NO:3; a PA with at least 95% amino acid sequence identity to the PA encoded by SEQ ID NO:1; a NP with at least 95% amino acid sequence identity to the NP encoded by SEQ ID NO:4; a M1 with at least 95% amino acid sequence identity to the M1 encoded by SEQ ID NO:5; or a NS1 or NS2 with at least 95% amino acid sequence identity to the NS1 or NS2 encoded by SEQ ID NO:6.

17. The isolated recombinant influenza virus of claim 1, wherein at least one of the PA, PB1, PB2, NP, NS, and M viral segments comprise: a PB1 with at least 95% amino acid sequence identity to the PB1 encoded by SEQ ID NO:10; a PB2 with at least 95% amino acid sequence identity to the PB2 encoded by SEQ ID NO:11; a PA with at least 95% amino acid sequence identity to the PA encoded by SEQ ID NO:12; a NP with at least 95% amino acid sequence identity to the NP encoded by SEQ ID NO:13; a M1 with at least 95% amino acid sequence identity to the M1 encoded by SEQ ID NO:14; or a NS1 or NS2 with at least 95% amino acid sequence identity to the NS1 or NS2 encoded by SEQ ID NO:15.

18. A vaccine having the isolated recombinant virus of claim 1.

19. A method to prepare influenza virus, comprising: contacting a cell with:
a vector for vRNA or cRNA production comprising a promoter operably linked to an influenza virus PA DNA linked to a transcription termination sequence, a vector for vRNA or cRNA production comprising a promoter operably linked to an influenza virus PB1 DNA linked to a transcription termination sequence, a vector for vRNA or cRNA production comprising a promoter operably linked to an influenza virus PB2 DNA linked to a transcription termination sequence, a vector for vRNA or cRNA production comprising a promoter operably linked to an influenza virus HA DNA linked to a transcription termination sequence, a vector for vRNA or cRNA production comprising a promoter operably linked to an influenza virus NP DNA linked to a transcription termination sequence, a vector for vRNA or cRNA production comprising a promoter operably linked to an influenza virus NA DNA linked to a transcription termination sequence, a vector for vRNA or cRNA production comprising a promoter operably linked to an influenza virus M DNA linked to a transcription termination sequence, and a vector for vRNA or cRNA production comprising a promoter operably linked to an influenza virus NS DNA linked to a transcription termination sequence, wherein the PB1, PB2, PA, NP, NS, and M DNAs in the vectors for vRNA or cRNA production are from one or more influenza vaccine virus isolates, wherein the NA DNA in the vector for vRNA or cRNA production of NA has sequences for a heterologous, recombinant or chimeric NA, and wherein the HA DNA in the vector for vRNA or cRNA production of HA has sequences for a heterologous, recombinant or chimeric HA, wherein the PB1 DNA encodes a PB1 polypeptide having a residue other than glycine or alanine at position 62, a residue other than lysine at position 577, a residue other than leucine or valine at position 624, and/or a residue other than methionine at position 640, or wherein the PB2 DNA encodes a PB2 polypeptide having a residue other than glutamine at position 439, and/or wherein the M DNA encodes a M1 polypeptide having a residue other than lysine at position 35, or a combination thereof; and
a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, or a vector for mRNA production comprising a promoter operably linked to a DNA segment encoding influenza virus NS2;
in an amount effective to yield infectious influenza virus.

20. The method of claim 19, wherein the cell is an avian cell.

21. The method of claim 19, wherein the cell is a mammalian cell.

22. The method of claim 21, wherein the cell is a Vero cell, a human cell or a MDCK cell.

23. The method of claim, 19 wherein the PB1, PB2, PA, NP, NS, and M DNAs in the vectors for vRNA or cRNA productions have a sequence that corresponds to one that encodes a polypeptide having at least 95% amino acid sequence identity to a corresponding polypeptide encoded by SEQ ID NOs:1-6 or 10-15.

24. The method of claim 19, further comprising isolating the virus.

25. The method of claim 19, wherein at least one of PA, PB1, or PB2 viral segments has a C to U promoter mutation.

26. Virus obtained by the method of claim 18.

27. A vector for vRNA or mRNA expression of a) influenza virus PB1 having at least 85% amino acid sequence identity to a polypeptide encoded by SEQ ID NO:2 and having a residue other than glycine or alanine at position 62, a residue other than lysine at position 577, a residue other than leucine or valine at position 624, and/or a residue other than methionine at position 640, or a combination thereof; b) influenza virus PB2 having at least 85% amino acid sequence identity to SEQ ID NO:3 and having a residue other than glutamine at position 439, or c) influenza virus M1 having at least 85% amino acid sequence identity to a M1 encoded by SEQ ID NO:6 and a residue other than lysine at position 35.

28. A method of immunizing an avian or a mammal comprising:
administering an effective amount of a composition comprising:
(a) a virus having PA, PB1, PB2, NP, NS, and M viral segments from a first influenza vaccine virus isolate, a heterologous, recombinant or chimeric influenza virus NA viral segment, and a heterologous, recombinant or chimeric HA viral segment, wherein the PB1 viral segment encodes a PB1 polypeptide having a residue other than glycine (G) or alanine (A) at position 62, a residue other than lysine (K) at position 577, a residue other than leucine (L) or valine (V) at position 624, and/or a residue other than methionine (M) at position 640, wherein the PB2 segment encodes a PB2 polypeptide having a residue other than glutamine (Q) at position 439, or wherein the M segment encodes a M1 polypeptide having a residue other than K at position 35; or a combination thereof; or
(b) a virus obtained by contacting a cell with:
a vector for vRNA or cRNA production comprising a promoter operably linked to an influenza virus PA DNA linked to a transcription termination sequence, a vector for vRNA or cRNA production comprising a promoter operably linked to an influenza virus PB1 DNA linked to a transcription termination sequence, a vector for vRNA or cRNA production comprising a promoter operably linked to an influenza virus PB2 DNA linked to a transcription termination sequence, a vector for vRNA or cRNA production comprising a promoter operably linked to an influenza virus HA DNA linked to a transcription termination sequence, a vector for vRNA or cRNA production comprising a promoter operably linked to an influenza virus NP DNA linked to a transcription termination sequence, a vector for vRNA or cRNA production comprising a promoter operably linked to an influenza virus NA DNA linked to a transcription termination sequence, a vector for vRNA or cRNA production comprising a promoter operably linked to an influenza virus M DNA linked to a transcription termination sequence, and a vector for vRNA or cRNA production comprising a promoter operably linked to an influenza virus NS DNA linked to a transcription termination sequence, wherein the PB1, PB2, PA, NP, NS, and M DNAs in the vectors for vRNA or cRNA production are from one or more influenza vaccine virus isolates, wherein the NA DNA in the vector for vRNA or cRNA production of NA has sequences for a heterologous, recombinant or chimeric NA, and wherein the HA DNA in the vector for vRNA or cRNA production of HA has sequences for a heterologous, recombinant or chimeric HA, wherein the PB1 DNA encodes a PB1 polypeptide having a residue other than glycine or alanine at position 62, a residue other than lysine at position 577, a residue other than leucine or valine at position 624, and/or a residue other than methionine at position 640, or wherein the PB2 DNA encodes a PB2 polypeptide having a residue other than glutamine at position 439, and/or wherein the M DNA encodes a M1 polypeptide having a residue other than lysine at position 35, or a combination thereof; and
a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, or a vector for mRNA production comprising a promoter operably linked to a DNA segment encoding influenza virus NS2;
in an amount effective to yield infectious influenza virus.

29. The method of claim 28 wherein the mammal is a human.
